(19) United States
(12) Patent Application Publication
(10) Pub. No.: US 2026/0315141 A1
(43) Pub. Date: Oct. 15, 2026

(54) DEVICE, SYSTEM, AND METHOD TO MAP AND ABLATE CARDIAC TISSUE USING AN EXPANDABLE BASKET CATHETER HAVING A CONTACT FORCE SENSOR

(71) Applicant: Biosense Webster (Israel) Ltd., Yokneam (IL)

(72) Inventors: Paras PARIKH, Irvine, CA (US); Christopher Thomas BEECKLER, Brea, CA (US); Tushar SHARMA, Mission Viejo, CA (US); Assaf GOVARI, Haifa (IL); Andres Claudio ALTMANN, Haifa (IL); Eric MANIRASITH, Irvine, CA (US); Terrence TROUTMAN, Irvine, CA (US); Justin George LICHTER, Irvine, CA (US)

(73) Assignee: Biosense Webster (Israel) Ltd., Yokneam (IL)

(21) Appl. No.: 19/557,014

(22) Filed: Mar. 4, 2026

Related U.S. Application Data
(60) Provisional application No. 63/768,666, filed on Mar. 7, 2025.

(51) Int. Cl. A61B 18/14; A61B 17/00; A61B 18/00; A61B 18/12; A61B 34/10; A61B 90/00

(52) U.S. Cl. CPC A61B 18/1492; A61B 18/1206; A61B 34/10; A61B 2017/00154; A61B 2018/0016; A61B 2018/00267; A61B 2018/00357; A61B 2018/00577; A61B 2018/00839; A61B 2090/065

(57) ABSTRACT

The disclosed technology includes a method comprising: navigating a medical probe to a target location in a patient's cardiovascular system, the medical probe extending along a longitudinal axis and comprising a plurality of spines configured to bow radially outward from the longitudinal axis, the plurality of spines comprising a plurality of electrodes disposed thereon; placing at least a portion of the plurality of electrodes in contact with tissue of the patient's cardiovascular system with an indicated contact force of about 20 grams or greater; delivering, via the at least a portion of the plurality of electrodes, a plurality of electrical pulses to the tissue, the plurality of electrical pulses being configured to ablate the tissue to form a plurality of lesions having a spacing of about less than or equal to 9 millimeters between approximate centers of adjacent lesions.

### REFERENCE TO RELATED APPLICATIONS

This application claims benefit of priority under 35 U.S.C. § 119 to prior filed U.S. Provisional Patent Application No. 63/768,666, filed Mar. 7, 2025 (Attorney Docket No.: 253757.000534 (BIO6981USPSP1)), the entire contents of which is hereby incorporated by reference as if set forth in full herein.

This application is related to U.S. Patent Publication Nos. 2023/0226638A1 (Attorney Docket No. BIO6567USNP1 (253757.000334)); 2023/0225787A1 (Attorney Docket No. BIO6568USNP1 (253757.000335)); 2023/0225783A1 (Attorney Docket No. BIO6569USNP1 (253757.000336)); 2023/0225788A1 (Attorney Docket No. BIO6570USNP1 (253757.000337)); 2023/0225784A1 (Attorney Docket No. BIO6571USNP1 (253757.000338)); 2023/0225789A1 (Attorney Docket No. BIO6572USNP1 (253757.000339)); 2023/0225790A1 (Attorney Docket No. BIO6591USNP1 (253757.000340)); 2023/0226336A1 (Attorney Docket No. BIO6592USNP1 (253757.000341)); 2023/0301707A1 (Attorney Docket No. BIO6638USNP1 (253757.000376)); 2023/0301712A1 (Attorney Docket No. BIO6639USNP1 (253757.000364)); 2023/0346461A1 (Attorney Docket No. BIO6640USNP1 (253757.000389)); 2023/0301713A1 (Attorney Docket No. BIO6641USNP1 (253757.000388)); 2024/0197391A1 (Attorney Docket No. BIO6642USNP1 (253757.000449)); 2023/0346463A1 (Attorney Docket No. BIO6643USNP1 (253757.000390)); 2023/0346464A1 (Attorney Docket No. BIO6675USNP1 (253757.000394)); 2024/0206761A1 (Attorney Docket No. BIO6692USNP1 (253757.000457)); 2023/0346459A1 (Attorney Docket No. BIO6693USNP1 (253757.000347)); 2023/0346455A1 (Attorney Docket No. BIO6693USNP3 (253757.000395)); 2024/0189023A1 (Attorney Docket No. BIO6735USNP1 (253757.000441)); 2024/0216048A1 (Attorney Docket No. BIO6737USNP1 (253757.000458)); 2024/0216051A1 (Attorney Docket No. BIO6738USNP1 (253757.000465)); 2023/0346462A1 (Attorney Docket No. BIO6739USNP1 (253757.000245)); 2024/0206965A1 (Attorney Docket No. BIO6741USNP1 (253757.000451)); 2024/0216045A1 (Attorney Docket No. BIO6743USNP1 (253757.000397)); 2023/0346466A1 (Attorney Docket No. BIO6744USNP1 (253757.000398)); 2024/0216075A1 (Attorney Docket No. BIO6745USNP1 (253757.000453)); 2024/0216056A1 (Attorney Docket No. BIO6746USNP1 (253757.000461)); 2024/0180615A1 (Attorney Docket No. BIO6747USNP1 (253757.000448)); 2024/0180614A1 (Attorney Docket No. BIO6748USNP1 (253757.000450)); 2024/0197392A1 (Attorney Docket No. BIO6750USNP1 (253757.000456)); 2023/0346465A1 (Attorney Docket No. BIO6794USNP1 (253757.000396)); 2024/0398469A1 (Attorney Docket No. BIO6816USNP1 (253757.000510)); 2024/0374305A1 (Attorney Docket No. BIO6834USNP1 (253757.000505)); 2024/0398468A1 (Attorney Docket No. BIO6841USNP1 (253757.000509)); 2025/0025230A1 (Attorney Docket No. BIO6842USNP1 (253757.000521)); 2024/0398351A1 (Attorney Docket No. BIO6844USNP1 (253757.000511)); and 2024/0382251A1 (Attorney Docket No. BIO6847USNP1 (253757.000507)), the entire contents and substance of each of which is incorporated herein by reference in its entirety and attached in the appendix included with priority U.S. Provisional Patent Application No. 63/768,666, filed Mar. 7, 2025 (Attorney Docket No.: 253757.000534 (BIO6981USPSP1)).

### FIELD

The present invention relates generally to devices, systems, and methods of mapping cardiac structures and ablating tissue using a catheter having a contact force sensor and configured to deliver pulses of energy to the tissue.

### BACKGROUND

Cardiac arrhythmias, such as atrial fibrillation (AF), occur when regions of cardiac tissue abnormally conduct electric signals to adjacent tissue. This disrupts the normal cardiac cycle and causes asynchronous rhythm. Certain procedures exist for treating arrhythmia, including surgically disrupting the origin of the signals causing the arrhythmia and disrupting the conducting pathway for such signals. By selectively ablating cardiac tissue by application of energy via a catheter, it is sometimes possible to cease or modify the propagation of unwanted electrical signals from one portion of the heart to another. Regions of cardiac tissue can be mapped by a catheter to identify the abnormal electrical signals and to determine proper location to ablate tissue.

Many current ablation approaches in the art tend to utilize thermal ablation techniques such as applying radiofrequency (RF) electrical energy to heat tissue or using cryoablation to cool the tissue to very low temperature. These methods, however, require a physician to be highly trained to ensure the ablation is completed effectively and safely due to the inherent risks of such procedures.

Other ablation approaches use irreversible electroporation (IRE) to ablate cardiac tissue using nonthermal ablation methods. IRE delivers short pulses of high voltage to tissues and generates an unrecoverable permeabilization of cell membranes. Delivery of IRE energy to tissues using multi-electrode catheters was previously proposed in the patent literature. Examples of systems and devices configured for IRE ablation are disclosed in U.S. Patent Pub. Nos. 2021/0161592A1, 2021/0169550A1, 2021/0169567A1, 2021/0169568A1, 2021/0177503A1, 2021/0186604A1, and 2021/0196372A1, each of which are incorporated herein by reference and attached in the appendix included with priority U.S. Provisional Patent Application No. 63/768,666, filed Mar. 7, 2025 (Attorney Docket No.: 253757.000534 (BIO6981USPSP1)).

Existing catheters and methods of using catheters, unfortunately, often require multiple applications of ablative energy and often require two different catheters-one to complete the mapping and another to complete the ablation. This elongates the ablation procedure and increases the chances of complications associated with insertion and removal of catheters. Furthermore, existing catheters systems often require more than one application of ablative energy due to the physician being unsure whether the catheter is in sufficient contact with the tissue. What is needed, therefore, are devices, systems, and methods of mapping and ablating tissue using a basket catheter having multiple electrodes and a contact force sensor as described further herein.

### SUMMARY

The disclosed technology can include a method of cardiac ablation comprising navigating a medical probe to a target location in a patient's cardiovascular system. The medical probe can be configured such that it extends along a longitudinal axis and comprises a plurality of spines configured to bow radially outward from the longitudinal axis. The plurality of spines can comprise a plurality of electrodes disposed thereon.

The method can further include placing at least a portion of the plurality of electrodes in contact with tissue of the patient's cardiovascular system with an indicated contact force of about 20 grams or greater and delivering, via the at least a portion of the plurality of electrodes, a plurality of electrical pulses to the tissue. The plurality of electrical pulses can be configured to ablate the tissue to form a plurality of lesions having a spacing of about less than or equal to 9 millimeters between approximate centers of adjacent lesions.

The disclosed technology can further include a medical system comprising a medical probe extending along a longitudinal axis. The medical probe can comprise a plurality of spines configured to bow radially outward from the longitudinal axis, a plurality of electrodes disposed along the plurality of spines, and a force sensor.

The medical system can further include a power generator, one or more processors, and a memory storing instructions that, when executed by the one or more processors, are configured to cause the medical system to determine contact force between the medical probe and tissue of a patient's cardiovascular system. In response to determining that the contact force is indicated at about 20 grams or greater, the instructions can further cause the power generator to output a plurality of electrical pulses to the tissue. The plurality of electrical pulses can be configured to ablate the tissue to form a plurality of lesions having a spacing of about less than or equal to 9 millimeters between approximate centers of adjacent lesions.

The disclosed technology can include an electrophysiology system configured to provide an efficacy index of a cardiac ablation treatment. The electrophysiology system can include a processor in communication with a medical probe, user interface, display, memory and storage. The processor can be configured to collect force data from the medical probe at a target location in a patient's cardiovascular system with the force data being indicative of a contact force applied to the medical probe at individual ablation sites of a plurality of ablation sites at the target location.

The processor can be further configured to collect ablation data corresponding to ablative energy applied at each individual ablation site of the plurality of ablation sites; determine a predicted ablation index; and determine a distance between at least two individual ablation sites of the plurality of ablation sites. The processor can be further configured to output to the display an efficacy index based at least in part on the force data, the predicted ablation index, and the distance between the at least two individual ablation sites. The efficacy index can comprise a probability of recurrence of one or more cardiovascular abnormalities.

The disclosed technology can further include a method of determining an efficacy index for a cardiac ablation procedure with a computerized electroanatomical mapping system that receives location and force data provided by a medical end effector disposed in a patient. The method can include calculating an ablation index from force data representative of contact force applied to the medical end effector in contact against biological tissue and ablation energy delivered to the biological tissue at a plurality of ablation sites.

The method can further include determining a distance between at least two individual ablation sites of the plurality of ablation sites and calculating an efficacy index based at least in part on the force data, the ablation index, and the distance between the at least two individual ablation sites. The efficacy index can comprise a probability of recurrence of one or more cardiovascular abnormalities. The method can also include displaying the efficacy index on a display of the mapping system.

Additional features, functionalities, and applications of the disclosed technology are discussed herein in more detail.

### BRIEF DESCRIPTION OF THE DRAWINGS

The above and further aspects of this disclosed technology are further discussed with reference to the following description in conjunction with the accompanying drawings, in which like numerals indicate like structural elements and features in various figures. The drawings are not necessarily to scale, emphasis instead being placed upon illustrating principles of the invention. The figures depict one or more implementations of the inventive devices, by way of example only, not by way of limitation.

This patent or application file contains at least one drawing executed in color. Copies of this patent or patent application publication with color drawings will be provided by the Office upon request and payment of the necessary fee.

FIG. 1 is a schematic pictorial illustration of a medical system including a medical probe whose distal end includes a basket assembly with electrodes, in accordance with an example of the disclosed technology;

FIG. 2 is a perspective view of a medical probe in an expanded form, in accordance with an example of the disclosed technology;

FIG. 3A illustrates the medical probe of FIG. 2 with only the underlying spine structure and one electrode disposed on one spine in accordance with an example of the disclosed technology;

FIG. 3B illustrates the spine structure as formed from a tube stock in accordance with an example of the disclosed technology;

FIG. 4 illustrates an exploded view of the medical probe of FIG. 2, in accordance with an example of the disclosed technology;

FIG. 5A illustrates a perspective view of a contact force sensor assembly and a spine retention hub of the medical probe, in accordance with an example of the disclosed technology;

FIGS. 5B and 5C illustrate exploded views of a contact force sensor assembly and a spine retention hub of the medical probe, in accordance with an example of the disclosed technology;

FIG. 6A illustrates a section view of a contact force sensor assembly and a spine retention hub of the medical probe, in accordance with an example of the disclosed technology;

FIG. 6B illustrates and exploded section view of a contact force sensor assembly and a spine retention hub of the medical probe, in accordance with an example of the disclosed technology;

FIGS. 7A-7C illustrate section and detail views of a contact force sensor assembly and a spine retention hub of the medical probe to illustrate how the contact force sensor assembly and spine retention hub are coupled together, in accordance with an example of the disclosed technology;

FIG. 8 is a schematic pictorial illustration showing a perspective view of another example medical probe in an expanded form, in accordance with another example of the disclosed technology;

FIG. 9 is a schematic pictorial illustration showing a perspective view of the medical probe of FIG. 8 showing the spines, in accordance with the disclosed technology;

FIG. 10 illustrates a method of determining an ablation index, in accordance with an example of the disclosed technology;

FIG. 11 is a schematic diagram of a catheter configured for mapping and ablation of tissue, in accordance with an example of the disclosed technology;

FIG. 12 is a schematic diagram of a basket assembly having a plurality of electrodes, in accordance with an example of the disclosed technology;

FIG. 13A is a schematic diagram of a system of mapping and ablating tissue, in accordance with an example of the disclosed technology;

FIG. 13B is a table indicating components of the system shown in FIG. 13A, in accordance with an example of the disclosed technology;

FIG. 13C is a schematic diagram of another view of the system shown in FIG. 13A, in accordance with an example of the disclosed technology;

FIG. 13D is a table indicating components of the system shown in FIG. 13C, in accordance with an example of the disclosed technology;

FIG. 14A is an illustration of a user interface of a system for mapping and ablating tissue, in accordance with an example of the disclosed technology;

FIG. 14B is a table indicating features of the user interface shown in FIG. 14A, in accordance with an example of the disclosed technology;

FIG. 15 is an illustration of another user interface used with a system for mapping and ablation tissue, in accordance with an example of the disclosed technology;

FIGS. 16A and 16B illustrate various views of an illustration of the end effector as displayed on a user interface, in accordance with an example of the disclosed technology;

FIG. 17 is a table indicating various components that can be used with a system for mapping and ablation tissue, in accordance with an example of the disclosed technology;

FIG. 18 is a chart illustrating various phases of a study of a catheter configured for mapping and ablating tissue, in accordance with an example of the disclosed technology;

FIGS. 19A and 19B are a table illustrating a schedule of events of the study, in accordance with an example of the disclosed technology;

FIGS. 20A, 20B, and 20C are a table of primary adverse events, in accordance with an example of the disclosed technology;

FIGS. 21A and 21B are a tables illustrating recommended parameters of completing a procedure with a catheter configured for mapping and ablating tissue, in accordance with an example of the disclosed technology;

FIG. 22 is a schematic diagram of an example of the various segments within a heart and a corresponding ablation scheme, in accordance with an example of the disclosed technology;

FIG. 23 illustrates baseline characteristics of participants in the study, in accordance with an example of the disclosed technology;

FIG. 24 illustrates the procedural characteristics of the safety analysis set, in accordance with an example of the disclosed technology;

FIG. 25A illustrates a diagram of ablation completed in a baseline procedure while FIG. 25B illustrates durable isolation in all four pulmonary veins, in accordance with an example of the disclosed technology;

FIG. 26 illustrates the spatial distribution of the locations of reconnections identified in a patient data set, in accordance with an example of the disclosed technology;

FIG. 27 illustrates an example of reconnection in a pulmonary vein after an ablation procedure, in accordance with an example of the disclosed technology;

FIG. 28 illustrates another example of reconnection in a pulmonary vein after an ablation procedure, in accordance with an example of the disclosed technology;

FIG. 29A illustrates various PF Indexes recorded during an ablation procedure of a first patient, in accordance with an example of the disclosed technology;

FIG. 29B illustrates average force values recorded during the ablation procedure of the first patient, in accordance with an example of the disclosed technology;

FIG. 29C illustrates the inter-lesion distances recorded during the ablation procedure of the first patient, in accordance with an example of the disclosed technology;

FIG. 30A illustrates an electroanatomical map recorded of the first patient's heart after the procedure performed with the recorded parameters shown and described in FIGS. 29A-29C, in accordance with an example of the disclosed technology;

FIG. 30B illustrates an electro anatomical map and electrogram recording shown the reconnection identified in FIG. 30A, in accordance with an example of the disclosed technology;

FIG. 31A illustrates various PF Indexes recorded during an ablation procedure of a second patient, in accordance with an example of the disclosed technology;

FIG. 31B illustrates average force values recorded during the ablation procedure of the second patient, in accordance with an example of the disclosed technology;

FIG. 31C illustrates the inter-lesion distances recorded during the ablation procedure of the second patient, in accordance with an example of the disclosed technology;

FIGS. 32A and 32B illustrate an electroanatomical map recorded of the second patient's heart after the procedure performed with the recorded parameters shown and described in FIGS. 31A-31C, in accordance with an example of the disclosed technology;

FIG. 33A illustrates various PF Indexes recorded during an ablation procedure of a third patient, in accordance with an example of the disclosed technology;

FIG. 33B illustrates average force values recorded during the ablation procedure of the third patient, in accordance with an example of the disclosed technology;

FIG. 33C illustrates the inter-lesion distances recorded during the ablation procedure of the third patient, in accordance with an example of the disclosed technology;

FIGS. 34A and 34B illustrate an electroanatomical map recorded of the third patient's heart after the procedure performed with the recorded parameters shown and described in FIGS. 33A-33C, in accordance with an example of the disclosed technology;

FIG. 35A illustrates various PF Indexes recorded during an ablation procedure of a fourth patient, in accordance with an example of the disclosed technology;

FIG. 35B illustrates average force values recorded during the ablation procedure of the fourth patient, in accordance with an example of the disclosed technology;

FIG. 35C illustrates the inter-lesion distances recorded during the ablation procedure of the fourth patient, in accordance with an example of the disclosed technology;

FIG. 36A illustrates various PF Indexes recorded during an ablation procedure of a fifth patient, in accordance with an example of the disclosed technology;

FIG. 36B illustrates average force values recorded during the ablation procedure of the fifth patient, in accordance with an example of the disclosed technology;

FIG. 36C illustrates the inter-lesion distances recorded during the ablation procedure of the fifth patient, in accordance with an example of the disclosed technology;

FIG. 37 is a flowchart of a method of determining an efficacy index, in accordance with an example of the disclosed technology; and

FIG. 38 illustrates a display of an electroanatomical map recorded of a patient's heart with an efficacy index also displayed, in accordance with an example of the disclosed technology.

### DETAILED DESCRIPTION

The disclosed technology includes a catheter device, a system, and associated methods that can be used to navigate to a patient's heart, map electrophysiological signals propagating through the tissue of the heart, and ablate the tissue of the heart. As will become apparent throughout this disclosure, the disclosed technology is configured to achieve effective ablation of the tissue while reducing the likelihood that repeat ablation will be needed to achieve effective isolation. In some examples, the disclosed technology includes a basket catheter with a plurality of electrodes and a contact force sensor. The disclosed technology can include applying pulsed field ablation (PFA) while applying the electrodes to the tissue with a predetermined pressure. The disclosed technology includes determining an ablation index (sometimes referred to as a PF index) that can help to provide feedback on the depth and quality of lesions formed by ablation applied to the tissue. The ablation index can be a value that is based at least in part on parameters such as the pressure applied to the tissue and the characteristics of the pulsed field ablation such as the duration, magnitude, amplitude, frequency, number of pulses, number of pulse trains, and type of ablation (monophasic, biphasic, etc.). Furthermore, the disclosed technology can include methods of ensuring lesions formed by the pulsed field ablation are spaced a predetermined distance apart to achieve effective ablation (e.g., pulmonary vein isolation (PVI)). In some examples, the procedure can be performed without using fluoroscopy. These and other benefits of the disclosed technology will become apparent throughout this disclosure.

The following detailed description should be read with reference to the drawings, in which like elements in different drawings are identically numbered. The drawings, which are not necessarily to scale, depict selected examples and are not intended to limit the scope of the invention. The detailed description illustrates by way of example, not by way of limitation, the principles of the invention. This description will clearly enable one skilled in the art to make and use the invention, and describes several examples, adaptations, variations, alternatives and uses of the invention, including what is presently believed to be the best mode of carrying out the invention.

As used herein, the terms “about” or “approximately” for any numerical values or ranges indicate a suitable dimensional tolerance that allows the part or collection of components to function for its intended purpose as described herein. More specifically, “about” or “approximately” may refer to the range of values ±20% of the recited value, e.g., “about 90%” may refer to the range of values from 71% to 110%.

As used herein, the terms “patient,” “host,” “user,” and “subject” refer to any human or animal subject and are not intended to limit the systems or methods to human use, although use of the subject invention in a human patient represents a preferred embodiment. In addition, vasculature of a “patient,” “host,” “user,” and “subject” can be vasculature of a human or any animal. It should be appreciated that an animal can be a variety of any applicable type, including, but not limited thereto, mammal, veterinarian animal, livestock animal or pet type animal, etc. As an example, the animal can be a laboratory animal specifically selected to have certain characteristics similar to a human (e.g., rat, dog, pig, monkey, or the like). It should be appreciated that the subject can be any applicable human patient, for example. As well, the term “proximal” indicates a location closer to the operator or physician whereas “distal” indicates a location further away from the operator or physician.

As discussed herein, “operator” can include a doctor, physician, surgeon, technician, scientist, or any other individual or delivery instrumentation associated with delivery of a multi-electrode catheter to a desired location in a patient or subject.

As discussed herein, the term “ablate” or “ablation”, as it relates to the devices and corresponding systems of this disclosure, refers to components and structural features configured to reduce or prevent the generation of erratic cardiac signals in the cells by utilizing non-thermal energy (or at least low thermal energy), such as irreversible electroporation (IRE), referred throughout this disclosure interchangeably as pulsed electric field (PEF) and pulsed field ablation (PFA). Ablating or ablation as it relates to the devices and corresponding systems of this disclosure is used throughout this disclosure in reference to non-thermal ablation (or low thermal ablation) of cardiac tissue for certain conditions including, but not limited to, arrhythmias, atrial flutter ablation, pulmonary vein isolation, supraventricular tachycardia ablation, and ventricular tachycardia ablation. The term “ablate” or “ablation” also includes known methods, devices, and systems to achieve various forms of bodily tissue ablation as understood by a person skilled in the relevant art.

As discussed herein, the terms “bipolar” and “unipolar” when used to refer to ablation schemes describe ablation schemes which differ with respect to electrical current path and electric field distribution. “Bipolar” refers to ablation scheme utilizing a current path between two electrodes that are both positioned at a treatment site; current density and electric flux density is typically approximately equal at each of the two electrodes. “Unipolar” (sometimes referred to as “monopolar”) refers to ablation scheme utilizing a current path between two electrodes where one electrode including a high current density and high electric flux density is positioned at a treatment site, and a second electrode including comparatively lower current density and lower electric flux density is positioned remotely from the treatment site.

As discussed herein, the terms “biphasic pulse” and “monophasic pulse” refer to respective electrical signals. “Biphasic pulse” refers to an electrical signal including a positive-voltage phase pulse (referred to herein as “positive phase”) and a negative-voltage phase pulse (referred to herein as “negative phase”). “Monophasic pulse” refers to an electrical signal including only a positive or only a negative phase. Preferably, a system providing the biphasic pulse is configured to prevent application of a direct current voltage (DC) to a patient. For instance, the average voltage of the biphasic pulse can be zero volts with respect to ground or other common reference voltage. Additionally, or alternatively, the system can include a capacitor or other protective component. Where voltage amplitude of the biphasic and/or monophasic pulse is described herein, it is understood that the expressed voltage amplitude is an absolute value of the approximate peak amplitude of each of the positive-voltage phase and/or the negative-voltage phase. Each phase of the biphasic and monophasic pulse preferably has a square shape including an essentially constant voltage amplitude during a majority of the phase duration. Phases of the biphasic pulse are separated in time by an interphase delay.

IRE (also referred to herein as PEF or PFA) as discussed in this disclosure is a low or non-thermal cell death technology that can be used for ablation of atrial arrhythmias. To ablate using IRE/PEF/PFA, biphasic voltage pulses are applied to disrupt cellular structures of the myocardium. The biphasic pulses are non-sinusoidal and can be tuned to target cells based on electrophysiology of the cells. In contrast, to ablate using RF, a sinusoidal voltage waveform is applied to produce heat at the treatment area, indiscriminately heating all cells in the treatment area. IRE therefore has the capability to spare adjacent heat sensitive structures or tissues which would be of benefit in the reduction of possible complications known with ablation or isolation modalities. Additionally, or alternatively, monophasic pulses can be utilized.

Electroporation can be induced by applying a pulsed electric field across biological cells to cause reversible (temporary) or irreversible (permanent) creation of pores in the cell membrane. The cells have a transmembrane electrostatic potential that is increased above a resting potential upon application of the pulsed electric field. While the transmembrane electrostatic potential remains below a threshold potential, the electroporation is reversible, meaning the pores can close when the applied pulse electric field is removed, and the cells can self-repair and survive. If the transmembrane electrostatic potential increases beyond the threshold potential, the electroporation is irreversible, and the cells become permanently permeable. As a result, the cells die due to a loss of homeostasis and typically die by programmed cell death or apoptosis, which is believed to leave less scar tissue as compared to other ablation modalities. Generally, cells of differing types have differing threshold potential. For instance, heart cells have a threshold potential of approximately 500 V/cm, whereas for bone it is 3000 V/cm. These differences in threshold potential allow IRE to selectively target tissue based on threshold potential.

The solution of this disclosure includes systems and methods for applying electrical signals from catheter electrodes positioned in the vicinity of myocardial tissue, preferably by applying a pulsed electric field effective to induce electroporation in the myocardial tissue. The systems and methods can be effective to ablate targeted tissue by inducing irreversible electroporation. In some examples, the systems and methods can be effective to induce reversible electroporation as part of a diagnostic procedure. Reversible electroporation occurs when the voltage applied with the electrodes is below the electric field threshold of the target tissue allowing cells to repair. Reversible electroporation does not kill the cells but allows a physician to see the effect of reversible electroporation on electrical activation signals in the vicinity of the target location. Example systems and methods for reversible electroporation is disclosed in U.S. Patent Publication 2021/0162210, the entirety of which is incorporated herein by reference and attached in the appendix included with priority U.S. Provisional Patent Application No. 63/768,666, filed Mar. 7, 2025 (Attorney Docket No.: 253757.000534 (BIO6981USPSP1)).

The pulsed electric field, and its effectiveness to induce reversible and/or irreversible electroporation, can be affected by physical parameters of the system and biphasic pulse parameters of the electrical signal. Physical parameters can include electrode contact area, electrode spacing, electrode geometry, etc. examples presented herein generally include physical parameters adapted to effectively induce reversible and/or irreversible electroporation. Biphasic pulse parameters of the electrical signal can include voltage amplitude, pulse duration, pulse interphase delay, inter-pulse delay, total application time, delivered energy, etc. In some examples, parameters of the electrical signal can be adjusted to induce both reversible and irreversible electroporation given the same physical parameters. Examples of various systems and methods of ablation including IRE are presented in U.S. Patent Publications 2021/0161592A1, 2021/0169550A1, 2021/0169567A1, 2021/0169568A1, 2021/0177503A1, 2021/0186604A1, and 2021/0196372A1, the entireties of each of which are incorporated herein by reference and attached in the appendix included with priority U.S. Provisional Patent Application No. 63/768,666, filed Mar. 7, 2025 (Attorney Docket No.: 253757.000534 (BIO6981USPSP1)).

To deliver pulsed field ablation (PFA) in an IRE (irreversible electroporation) procedure, electrodes should contact the tissue being ablated with a sufficiently large surface area. As described hereinbelow, the medical probe includes a tubular shaft including proximal and distal ends, and a basket assembly at the distal end of the tubular shaft. The basket assembly can include a plurality of linear spines and one or more electrodes coupled to each of the spines. The plurality of linear spines can converge at a central spine intersection including one or more cutouts. The cutouts can allow for bending of each spine such that the spines form an approximately spherical or oblate-spheroid basket assembly. It is noted that the cutouts (in various configurations described and illustrated in the specification) allows the basket to be compressed into a much smaller form factor when undeployed (or undergoing a retraction into a delivery sheath) without buckling or plastic deformation.

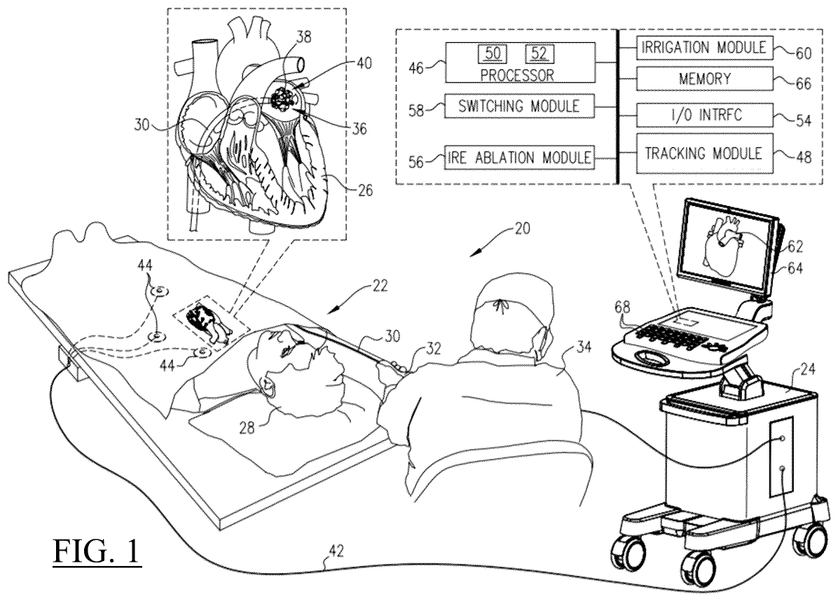

FIG. 1 is a schematic, pictorial illustration of a medical system 20 including a medical probe 22 and a control console 24, in accordance with an example of the present invention. Medical system 20 may be based, for example, on the CARTO® system, produced by Biosense Webster Inc. of 31 Technology Drive, Suite 200, Irvine, CA 92618 USA. In examples described hereinbelow, medical probe 22 can be used for diagnostic or therapeutic treatment, such as for performing ablation procedures in a heart 26 of a patient 28. Alternatively, medical probe 22 may be used, mutatis mutandis, for other therapeutic and/or diagnostic purposes in the heart or in other body organs.

Medical probe 22 includes a flexible insertion tube 30 and a handle 32 coupled to a proximal end of the tubular shaft. During a medical procedure, a medical professional 34 can insert probe 22 through the vascular system of patient 28 so that a distal end 36 of the medical probe 22 enters a body cavity such as a chamber of heart 26. Upon distal end 36 entering the chamber of heart 26, medical professional 34 can deploy a basket assembly 38 near the distal end 36 of the medical probe 22. Basket assembly 38 can include a plurality of electrodes 40 affixed to a plurality of spines 214. To start performing a medical procedure such as irreversible electroporation (IRE) ablation, medical professional 34 can manipulate a handle (either handle 32 or a separate handle, or both) to position distal end 36 so that electrodes 40 engage cardiac tissue at a desired location or locations. Upon positioning the distal end 36 so that electrodes 40 (disposed on the basket assembly 38) engage cardiac tissue, the medical professional 34 can activate the medical probe 22 such that electrical pulses are delivered by the electrodes 40 to perform the IRE ablation.

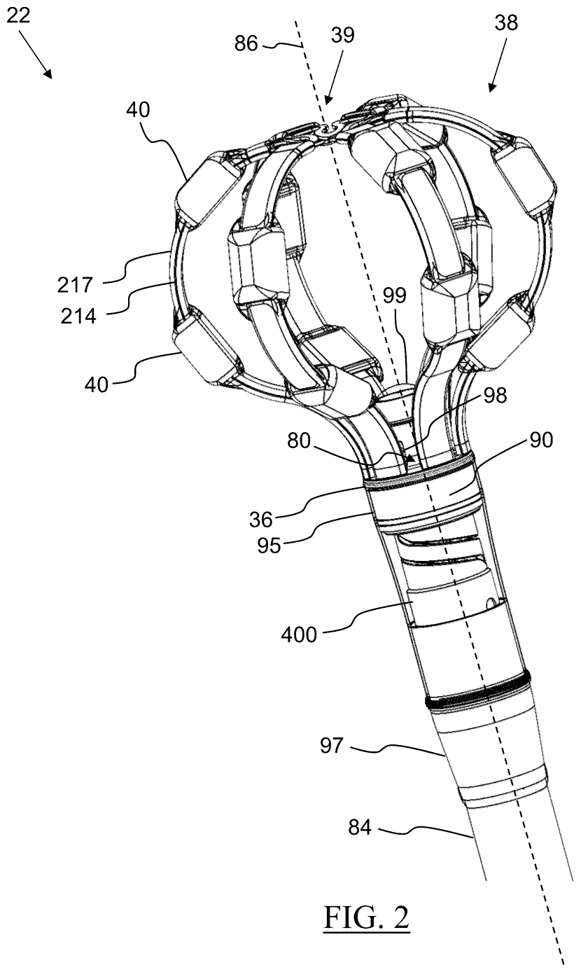

The medical probe 22 can include a guide sheath and a therapeutic catheter, wherein the guide sheath includes the flexible insertion tube 30 and the handle 32 and the therapeutic catheter includes the basket assembly 38, electrodes 40, a tubular shaft 84 (see FIGS. 2 through 4). The therapeutic catheter is translated through the guide sheath so that the basket assembly 38 is positioned in the heart 26. The distal end 36 of the medical probe 22 corresponds to a distal end of the guide sheath when the basket assembly 38 is contained within the flexible insertion tube 30, and the distal end 36 (of the tube 30) corresponds to a proximal portion (FIG. 2) of the basket assembly 38 when the basket assembly 38 is extended from the distal end of the guide sheath. The medical probe 22 can be alternatively configured to include a second handle on the therapeutic catheter and other features as understood by a person skilled in the pertinent art.

In the configuration shown in FIG. 1, control console 24 is connected, by a cable 42, to body surface electrodes, which typically include adhesive skin patches 44 that are affixed to patient 28. Control console 24 includes a processor 46 that, in conjunction with a tracking module 48, determines location coordinates of distal end 36 inside heart 26. Location coordinates can be determined based on electromagnetic position sensor output signals provided from the distal portion of the catheter when in the presence of a generated magnetic field. Location coordinates can additionally, or alternatively be based on impedances and/or currents measured between adhesive skin patches 44 and electrodes 40 that are affixed to basket assembly 38. In addition to being used for recording ECG signals or acting as location sensors during a medical procedure, electrodes 40 may perform other tasks such as ablating tissue in the heart.

As described hereinabove, in conjunction with tracking module 48, processor 46 may determine location coordinates of distal end 36 for tube 30 inside heart 26 based on impedances and/or currents measured between adhesive skin patches 44 and electrodes 40. Such a determination is typically after a calibration process relating the impedances or currents to known locations of the distal end has been performed. While examples presented herein describe electrodes 40 that are preferably configured to deliver IRE ablation energy to tissue in heart 26, configuring electrodes 40 to deliver any other type of ablation energy to tissue in any body cavity is considered to be within the spirit and scope of the present invention. Furthermore, although described in the context of being electrodes 40 that are configured to deliver IRE ablation energy to tissue in the heart 26, one skilled in the art will appreciate that the disclosed technology can be applicable to electrodes used for mapping electrophysiological signals and/or determining various characteristics of an organ or other part of the patient's 28 body.

Processor 46 may include real-time noise reduction circuitry 50 typically configured as a field programmable gate array (FPGA), followed by an analog-to-digital (A/D) signal conversion integrated circuit 52. The processor can be programmed to perform one or more algorithms and uses circuitry 50 and circuit 52 as well as features of modules to enable the medical professional 34 to perform the IRE ablation procedure.

Control console 24 also includes an input/output (I/O) communications interface 54 that enables control console 24 to transfer signals from, and/or transfer signals to electrodes 40 and adhesive skin patches 44. In the configuration shown in FIG. 1, control console 24 additionally includes an IRE ablation module 56 and a switching module 58.

IRE ablation module 56 is configured to generate IRE pulses including peak power in the range of tens of kilowatts. In some examples, the electrodes 40 are configured to deliver electrical pulses including a peak voltage of at least 1000 volts (V). The medical system 20 performs IRE ablation by delivering IRE pulses to electrodes 40. Preferably, the medical system 20 delivers biphasic pulses between electrodes 40 on the spine. Additionally, or alternatively, the medical system 20 delivers monophasic pulses between at least one of the electrodes 40 and a skin patch. The IRE ablation module 56 can be configured to generate IRE pulses in accordance with the technology disclosed in U.S. Pat. No. 11,540,877 filed Dec. 3, 2019 and issued on Jan. 3, 2023, the entirety of which is incorporated herein by reference and attached in the appendix included with priority U.S. Provisional Patent Application No. 63/768,666, filed Mar. 7, 2025 (Attorney Docket No.: 253757.000534 (BIO6981USPSP1)).

In order to prevent blood coagulation, system 20 supplies irrigation fluid (e.g., a normal saline solution) to distal end 36 of tube 30 and to the proximal area of basket assembly 38. It is noted that irrigation fluid can be supplied through the flexible insertion tube 30 or through a separate tube. Control console 24 includes an irrigation module 60 to monitor and control irrigation parameters, such as the pressure and the temperature of the irrigation fluid. It is noted that while the preference for the exemplary embodiments of the medical probe is for IRE or PFA, it is within the scope of the present invention to also use the medical probe separately only for RF ablation (unipolar mode with an external grounding electrode or bipolar mode) or in combination with IRE and RF ablations sequentially (certain electrodes in IRE mode and other electrodes in RF mode) or simultaneously (groups of electrodes in IRE mode and other electrodes in RF mode).

Based on signals received from electrodes 40 and/or adhesive skin patches 44, processor 46 can generate an electroanatomical map 62 that shows the location of distal end 36 in the patient's body. During the procedure, processor 46 can present map 62 to medical professional 34 on a display 64, and store data representing the electroanatomical map in a memory 66. Memory 66 may include any suitable volatile and/or non-volatile memory, such as random-access memory or a hard disk drive.

In some examples, medical professional 34 can manipulate map 62 using one or more input devices 68. In alternative examples, display 64 may include a touchscreen that can be configured to accept inputs from medical professional 34, in addition to presenting map 62.

FIG. 2 is an illustration of a prototype showing a perspective view of a medical probe 22 including a basket assembly 38 in an expanded form when unconstrained, such as by being advanced out of an insertion tube lumen 80 at a distal end 36 of an insertion tube 30. Probe 22 may include a contact force sensor assembly 400 to determine contact force of the spines against cardiac tissues. It should be noted that the medical probe 22 illustrated in FIG. 2 lacks the guide sheath illustrated in FIG. 1. In the expanded form FIG. 2, spines 214 bow radially outwardly and in the collapsed form not shown the spines 214 are arranged generally along a longitudinal axis 86 of insertion tube 30. For simplicity, only a single spine 214 has been marked with a reference number, but one of skill in the art will appreciate that the basket assembly 38 can include one or more spines 214 as illustrated in FIG. 2. Similarly only electrodes 40 and an insulative sleeve 217 on the single spine 214 are marked with references numbers, but one of skill in the art will appreciate that the basket assembly 38 can include one or more electrodes 40 and one or more insulative sleeves depending on the particular configuration. A plurality of electrically insulative sleeves 217 can be provided so that each jacket can be disposed between a respective spine 214 of the plurality of spines 214 and a respective electrode 40 of the plurality of electrodes 40, thereby electrically isolating the plurality of electrodes 40 from the plurality of spines 214.

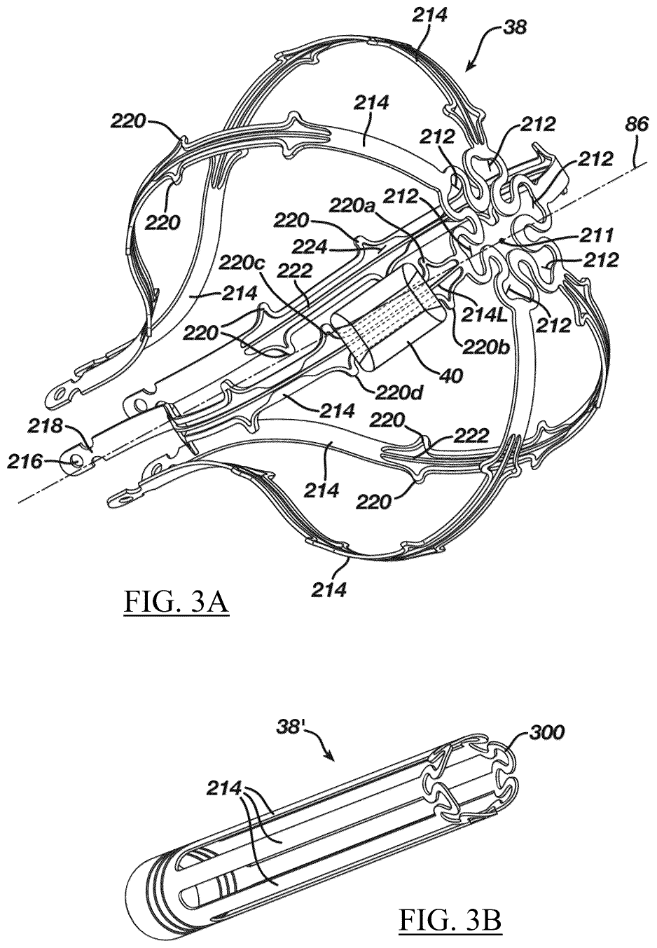

As shown in FIG. 2, basket assembly 38 includes a plurality of flexible spines 214 that are formed at the end of a tubular shaft 84 and are connected at both ends. During a medical procedure, medical professional 34 can deploy basket assembly 38 by extending tubular shaft 84 from insertion tube 30 causing basket assembly 38 to exit insertion tube 30 and transition to the expanded form. Spines 214 can have elliptical (e.g., circular), or rectangular cross-sections that may appear to be flat cross-sections, and include a flexible, resilient material (e.g., a shape-memory alloy such as nickel-titanium, also known as Nitinol) forming a strut as will be described in greater detail herein. As shown in FIGS. 2 and 3, basket assembly 38 has a proximal portion with a distal end 39. The medical probe 22 can include a spine retention hub 90 that extends longitudinally from a distal end of tubular shaft 84 towards distal end 39 of basket assembly 38. As described supra, control console 24 includes irrigation module 60 that delivers irrigation fluid to basket assembly 38 through tubular shaft 84.

Turning to FIG. 3A, the plurality of flexible linear spines 214 converge at a central spine intersection 211 that is also disposed on a longitudinal axis 86 defined by the spines 214. In some examples central spine intersection 211 can include one or more cutouts 212 that allow for bending of the spines 214 when each spine respective attachment end 216 is connected to the spine retention hub 90.

As shown herein, electrodes 40 positioned on spines 114 of basket assembly 38 can be configured to deliver ablation energy RF and/or IRE to tissue in heart 26. Additionally, or alternatively, the electrodes can also be used to determine the location of basket assembly 38 and/or to measure a physiological property such as local surface electrical potentials at respective locations on tissue in heart 26. The electrodes 40 can be biased such that a greater portion of the one or more electrodes 40 face outwardly from basket assembly 38 such that the one or more electrodes 40 deliver a greater amount of electrical energy outwardly away from the basket assembly 38 i.e., toward the heart 26 tissue than inwardly.

Examples of materials ideally suited for forming electrodes 40 include gold, platinum, and palladium and their respective alloys. These materials also have high thermal conductivity which allows the minimal heat generated on the tissue i.e., by the ablation energy delivered to the tissue to be conducted through the electrodes to the back side of the electrodes i.e., the portions of the electrodes on the inner sides of the spines, and then to the blood pool in heart 26.

Referring to FIG. 3A, basket assembly 38 of medical probe 22 is shown without the insulative sleeve 217 or associated wirings to electrodes 40 being disposed inside sleeve 217 to show the novel underlying basket structure 38. Basket 38 can include a single unitary structure that includes a plurality of spines 214 formed from a cylindrical tube stock, as illustrated in FIG. 3B, and treated to cause the spines 214 to bias radially outward. The material for the spine can be selected from a group consisting of nitinol, cobalt chromium, stainless steel, titanium, and combinations hereof.

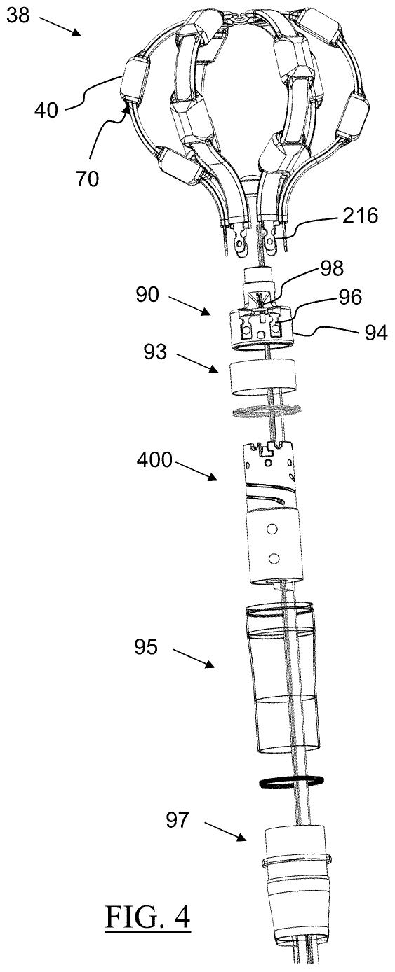

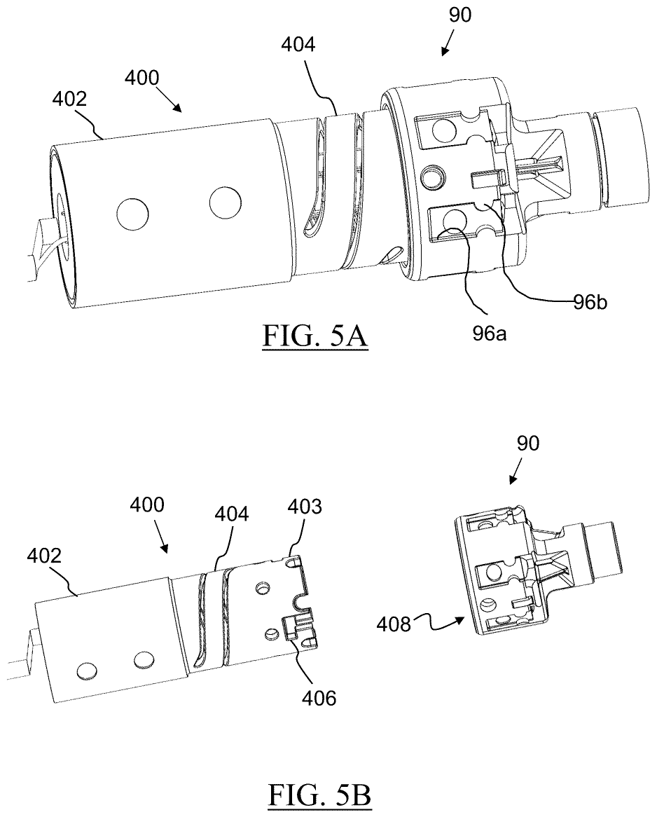

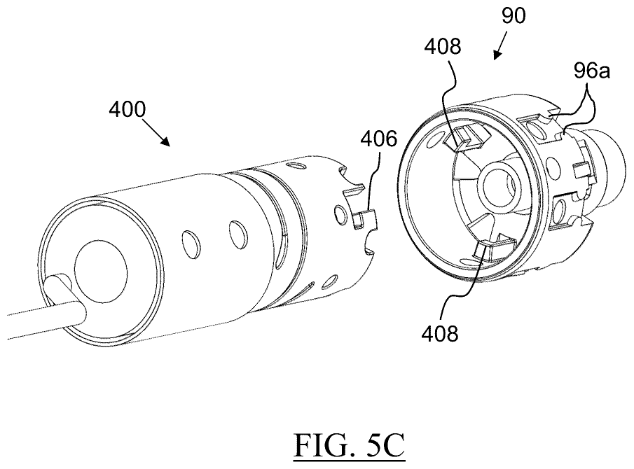

FIG. 4 illustrates an exploded view of the medical probe of FIG. 2, in accordance with an example of the disclosed technology. As illustrated in FIG. 4, the spine retention hub 90 can be inserted into the tubular shaft 84 and attached to the contact force sensor assembly 400. Spine retention hub 90 can include a cylindrical member 94 including a plurality of relief slots 96, multiple irrigation openings 98, and at least one spine retention hub electrode 99 (illustrated in FIG. 2) such as a position sensor or reference electrode, or some combination thereof. Relief slots 96 can be disposed on the outer surface of cylindrical member 94 and configured to allow a portion of each spine 214, such as each spine attachment end 216, to be fitted into a respective relief slot 96 of retention hub 90. The relief slot 96 is provided with undercuts 96a (FIG. 5A) running along the longitudinal axis to allow for insertion of spine attachment end 216 (which may have a hole as shown in FIG. 3A). Relief slot 96 may be provided with tabs 96b (FIG. 5C) that engage with complementary recess 218 on spine attachment end 216 (FIG. 3A) to prevent any twisting of the spine attachment end 216 of each spine 214 with respect to the retention hub 90. This configuration of the attachment ends 216 allow the multiple spines 214 at the proximal portion of the basket 38 to act as a single structural member with the retention hub 90. The spine retention hub 90 can also act as a coupler for a contact force sensor assembly 400 as described in greater detail herein. The attachment end 216 can be a generally linear end of the spine 214. The attachment end 216 can be configured to extend outwardly from the spine retention hub 90 such that the basket assembly 38 is positioned outwardly from the spine retention hub 90 and, consequently, outwardly from the tubular shaft 84. In this way, the spine 214 can be configured to position the basket assembly 38 distally from the distal end of the tubular shaft 84 and distal from the distal end of the insertion tube 30 when the basket assembly 38 is deployed.

Electrode 40 can be located substantially in place with respect to spine 214 by way of a retention member 220 formed integrally with the spine 214. In FIG. 3A, it can be seen that each of the spines 214 can include at least one retention member 220 extending generally transverse to the spine 214. To allow for insertion of the spine 214 through a lumen 70 extending through the electrode 40, each spine 214 can be bisected with a central spine member 222 so that empty space 224 is provided to allow retention member 220 to bend inwardly toward the central spine member 222. The shape of retention member 220 can be of any shape as along as such shape serves to allow the retention member 220 to be compressed for insertion into lumen 70 of electrode 40 and once released to prevent movement of electrode 40 with respect to the retention member. In one example, the at least one retention member 220 is shaped in a bow-like configuration with a center of such bow extending away from a periphery of spine 214. In a preferred embodiment, the at least one retention member 220 for each electrode 40 can include two bow-shaped members disposed in opposite directions and oriented transverse to a longer length 214L of each spine 214.

In the configuration shown in FIG. 3A, the at least one retention member may have first 220a, 220b and second sets 220c, 220d of retention members 220 spaced apart along the spines. The first set includes two bow-shaped members 220a, 220b disposed in opposite direction and transverse to a longer length 214L of each spine 214 and the second set includes two bow-shaped members 220c, 220d disposed in opposite direction and transverse to a longer length 214L of each spine 214 so that each electrode 40 is captured between the first and second sets of retention members 220a, 220b and 220c and 220d. FIG. 3B shows the basket assembly 38 as formed from a tube stock. It is also within the scope of this invention for the basket assembly 38 to be formed from a flat sheet stock, cut, and heat treated to achieve the spheroidal basket shape shown herein.

FIG. 4 illustrates an exploded view of the medical probe 22 of FIG. 2, in accordance with an example of the disclosed technology. Moving from top to bottom in FIG. 4, the assembly of the medical probe 22 will now be briefly described. As illustrated in FIG. 4, and as previously described, the spines 214 can be attached to the spine retention hub 90 to form the basket assembly 38. A spine retention sleeve 93 can be disposed over the spine retention hub to help secure the spines 214 in place. As will be described in greater detail herein, the contact force sensor assembly 400 can be coupled to the spine retention hub 90. The medical probe 22 can further include a contact force sensor assembly 400 that can be disposed in a contact force sensor assembly sleeve 95. The contact force sensor assembly sleeve 95 can be coupled to a proximal coupler 97 that can be coupled to the tubular shaft 84. When fully assembled, the basket assembly 38 can be attached to the tubular shaft 84 with the components just described to enable a medical professional 34 to insert the medical probe 22 into the heart 26 of a patient 28.

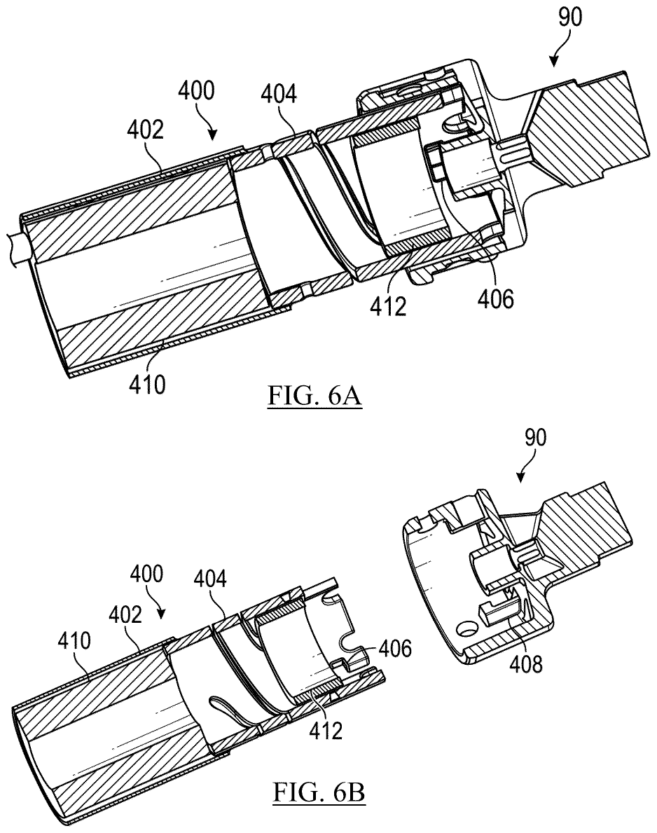

Turning now to FIGS. 5A-6B the contact force sensor assembly 400 and a spine retention hub 90 of the medical probe 22 will now be further described. Examples of contact force sensor assemblies are disclosed in U.S. Pat. Nos. 8,357,152 and 10,688,278 and U.S. Patent Pub. No. 2021/0187254A1, each of which are incorporated herein by reference and attached in the appendix included with priority U.S. Provisional Patent Application No. 63/768,666, filed Mar. 7, 2025 (Attorney Docket No.: 253757.000534 (BIO6981USPSP1)). FIG. 5A illustrates a perspective view of a contact force sensor assembly 400 and a spine retention hub 90 of the medical probe 22 coupled together while FIGS. 5B and 5C illustrate exploded views of the contact force sensor assembly 400 and the spine retention hub 90. Furthermore, FIGS. 6A and 6B illustrate section views of the contact force sensor assembly 400 and the spine retention hub 90.

As illustrated, the contact force sensor assembly 400 can include a proximal end 402 and a distal end 403. The proximal end 402 can house a magnetic field generator coil 410 and the distal end 403 can house a magnetic field sensor 412. As will be appreciated, the magnetic field generator coil 410 can be configured to generate a magnetic field while the magnetic field sensor 412 can be configured to detect the presence and magnitude of the magnetic field.

The contact force sensor assembly 400 can further include a deflection portion 404 disposed between the proximal end 402 and the distal end 403. The deflection portion 404 can be configured to deflect when a force is applied to the contact force sensor assembly 400. In other words, the deflection portion 404 can be configured to permit the proximal end 402 and the distal end 403 of the contact force sensor assembly 400 to move closer to each other when a force is applied to the contact force sensor assembly 400. In one example, the deflection portion 404 can comprise a helical spring formed into a body of the contact force sensor assembly 400 as illustrated in FIGS. 5A-6B. For example, helical cuts can be made in the body of the contact force sensor assembly 400 to form a helical spring. In this way, the body of the contact force sensor assembly 400 can itself form a spring without the need for additional components. In other examples, a spring can be assembled between the proximal end 402 and the distal end 403 to form the contact force sensor assembly 400. The contact force sensor assembly 400 can be disposed inside tubular shaft 84 and proximally in relation to the basket assembly 38 and as close as possible to the basket assembly 38 so that contact with cardiac tissue by the spines 214 can be transmitted to the contact force sensor assembly 400.

As will be appreciated, when the proximal end 402 is moved closer to the distal end 403 when a force is applied to the contact force sensor assembly 400, the magnetic field sensor 412 can detect a change in the magnitude of the force of the magnetic field generated by the magnetic field generator coil 410. Because the spring constant K of the deflection portion 404 can be predetermined and the distance between the magnetic field generator coil 410 and the magnetic field sensor 412 can be detected, the force applied to the medical probe 22 can be determined (e.g., by using Hooke's law, or the equation F=d*K). Furthermore, the contact force sensor assembly 400 can receive electrical signals from, and provides electrical signals to, console 24, to process received signals and determine forces, e.g., sub-gram forces, exerted on the basket assembly 38.

As illustrated in FIGS. 5A-6B, the contact force sensor assembly 400 can include a female connector 406 while the spine retention hub 90 can include a male connector 408. As will be appreciated, however, although shown and described for convenience as the contact force sensor assembly 400 having the female connector 406 and the spine retention hub 90 having the male connector 408, the two components can be switched around without departing from the scope of this disclosure. In other words, the contact force sensor assembly 400 can include the male connector 408 while the spine retention hub 90 can include the female connector 406 depending on the particular configuration. As will be appreciated, the contact force sensor assembly 400 can include a plurality of female connectors 406 while the spine retention hub 90 can include a plurality of male connectors 408. Alternatively, the contact force sensor assembly 400 can include both a female connector 406 and a male connector 408 while the spine retention hub can include a complimentary female connector 406 and a complimentary male connector 408.

The female connector 406 and the male connector 408 can form a bayonet mount configuration in which the male connector 408 can interlock with the female connector to couple the contact force sensor assembly 400 to the spine retention hub 90. Stated otherwise, the female connector 406 can comprise a slot forming a generally “L” shape and the male connector 408 can comprise a protrusion forming a generally complimentary “L” shape. In other words, the female connector 406 can include a slot having a first slot portion extending generally longitudinally into the contact force sensor assembly 400 from a distal end 403 of the contact force sensor assembly 400 and a second slot portion extending generally transversely from an end of the first slot portion. Similarly, the male connector 408 can include a protrusion having a first protrusion portion extending generally longitudinally away from the spine retention hub 90 and a second protrusion portion extending generally transversely from an end of the first protrusion portion.

When the male connector 408 is inserted into the female connector 406, the “L” shapes of the female connector 406 and the male connector 408 can interlock, thus causing the contact force sensor assembly 400 to be coupled to the spine retention hub 90. In some examples, the male connector 408 can be caused to spring forward into the female connector 406 when the “L” portions of the female connector 406 and the male connector 408 are aligned. In this way, the male connector 408 must be pushed to remove the male connector 408 from the female connector 406 (rather than simply twisting the contact force sensor assembly 400 and/or the spine retention hub 90.

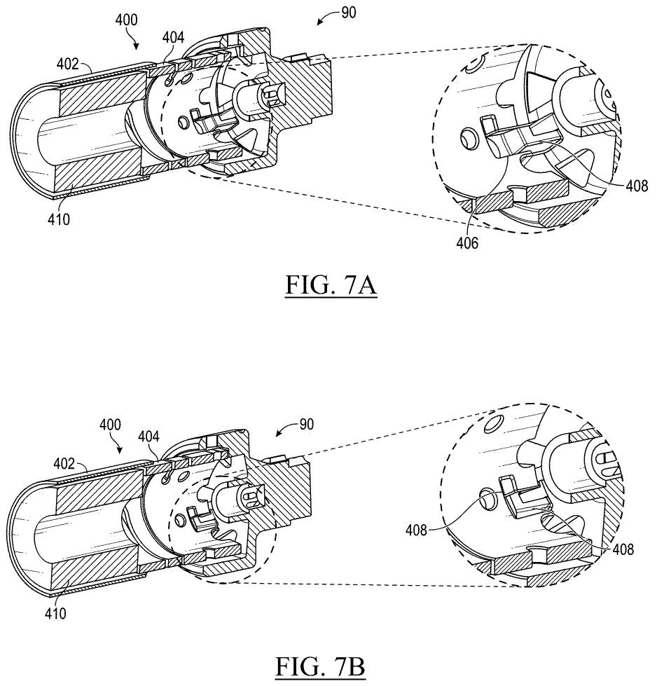

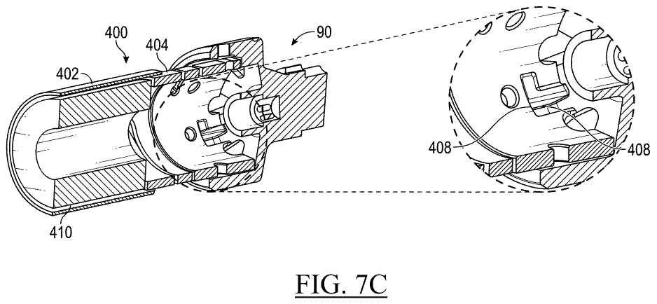

Turning now to FIGS. 7A-7C, the contact force sensor assembly 400 can be coupled to the spine retention hub 90 by aligning the female connector 406 with the male connector 408 (as illustrated in FIG. 7A), inserting the male connector 408 into the female connector 406 (as illustrated in FIG. 7B), and then twisting the contact force sensor assembly 400 and/or the spine retention hub 90 to cause the female connector 406 and the male connector 408 to interlock. In this way, the contact force sensor assembly 400 can be coupled to the spine retention hub 90 and prevent the contact force sensor assembly 400 from becoming dislodged from the spine retention hub 90 when the medical probe 22 is pulled through the flexible insertion tube 30.

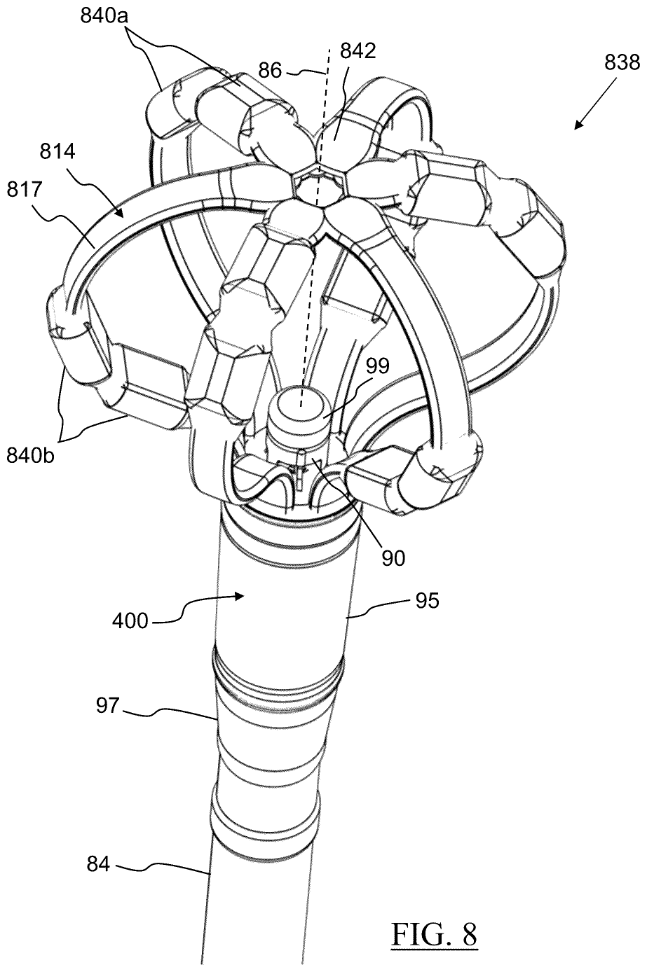

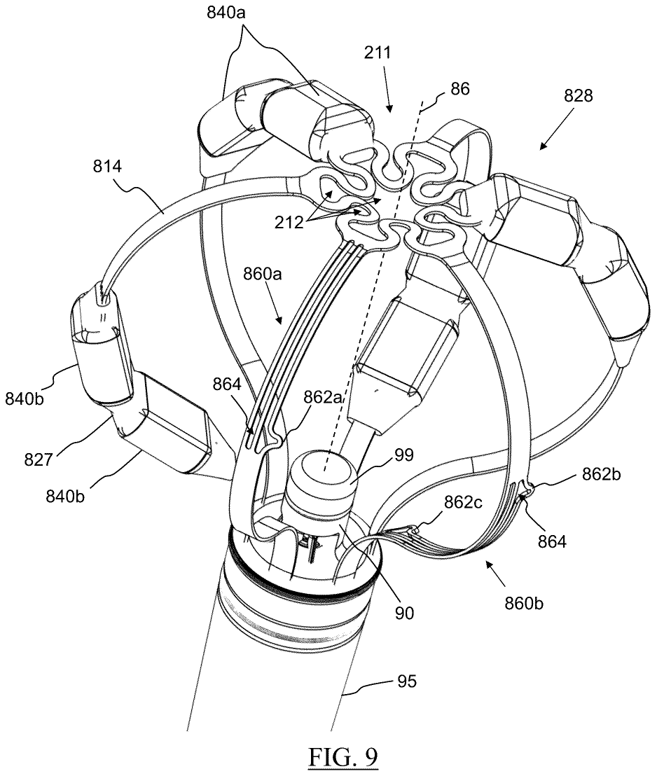

FIGS. 8 and 9 illustrate another example basket assembly 838 having a plurality of electrodes 840a, 840b disposed on spines 814 and a spine retention hub 90 having a spine retention hub electrode 99 mounted thereon. As shown in FIG. 8, the electrodes 840a, 840b can be disposed in alternating groupings of distal electrodes 840a and proximal electrodes 840b on adjacent spines 814. For example, and as shown in FIGS. 8 and 9, two electrodes 840a, 840b can be disposed on the spines 814 close to each other with no additional electrodes 840 disposed on the same spine 814. On a first spine 814, the two electrodes 840b can be disposed together near the proximal end of the spine 814 while on a second, adjacent spine 814 two electrodes 840a can be disposed together near the distal end of the adjacent spines 814. In this way, the electrodes 840a, 840b can be offset around the circumference of the basket assembly 838 such that the basket assembly 838 is better able to collapse when retracted into a sheath. When the basket assembly 838 is collapsed, the distal electrodes 840a are positioned entirely in a distal direction from the proximal electrodes 840b with a gap along the longitudinal axis 86 between the proximal electrodes 840b and the distal electrode 840a.

With the configuration of electrodes 840a, 840b disposed on the spines 814 as shown in FIGS. 8 and 9, the medical system 20 can be configured to output bipolar high-voltage DC pulses as may be used to effect irreversible electroporation (IRE) between the two adjacent electrodes 840a, 840b on a given spine 814, electrically connect the two adjacent electrodes 840 on a given spine 814 and output bipolar high-voltage DC pulses between one or more electrodes 840a, 840b on another one of the spines 814 of the basket assembly 838, and/or output monopolar high-voltage DC pulses between one or more of the electrodes 828 and the one or more adhesive skin patches 44 disposed on the patient's 28 skin. The two electrodes 840a, 840b on a given spine 814 can include an insulative material 827 disposed between the two electrodes 840a 840b, thereby electrically isolating the two electrodes 840a 840b from each other.

As shown in FIG. 8, the spines 814 can be covered with an insulative liner 817 that can be disposed between the electrodes 840 and the spines 814. The insulative liner 817 can electrically isolate the electrodes 840 from the spines 814 to prevent arcing or shorting to the spines 814. The insulative liner 817 can extend from the spine retention hub 90 to a distal end of the basket assembly 838. Furthermore, the insulative liner 817 can include flared ends 842 that can extend over at least a portion of the central spine intersection 211. In this way, the insulative liner 817 can have an atraumatic tip to prevent injury to tissue.

FIG. 9 is an illustration of the basket assembly 838 with the insulative liners 817, a pair of distal electrodes 840a, and a pair of proximal electrodes 840b, and other spine elements removed, for the sake of illustration, so that the frame of the basket assembly 838 is visible. As shown in FIG. 9, the spines 814 can extend from the spine retention hub 90 and be joined together at a central spine intersection 211. The central spine intersection 211 can include one or more cutouts 212 that can allow for bending of the spines 814. The spines 814 can further include an electrode retention region 860a, 860b that is configured to prevent an electrode 840a, 840b from sliding proximally or distally along the spine 814. As shown in FIG. 9, a first spine 814 can have distal spine retention region 860a and an adjacent spine can have a proximal spine retention region 860b. In this way, the spine retention regions 860a, 860b can be alternating between a proximal position and a distal position along the spines 814. That is, a first spine 814 can have an electrode retention region 860b disposed near a proximal end of the spine 814 and an adjacent spine 814a can have an electrode retention region 860 disposed near a distal end of the spine 814.

Each electrode retention region 860 can include one or more cutouts 864 that can permit the spine 814 to be bent or pinched inwardly. Each electrode retention region 860 can further include one or more retention members 862 that protrude outwardly and can be configured to prevent the electrode 840 from sliding proximally or distally along the spine 814. During manufacture, proximal ends of the frame of the basket assembly 838 are inserted into lumens of the electrodes 840a, 840b, and the electrodes 840a, 840b are slid distally along the spines 814 to their respective final position. The cutouts 864 permit the electrodes 840a, 840b to slide over a retention members 862a-c. Because of the one or more cutouts 864 in the spines 814, the retention members 862a-c can be configured to move inwardly when the spine 814 is pinched inwardly to permit an electrode 840a, 840b to slide over the retention member 862a-c. Once the electrode 840a, 840b is slid past the retention member 862, the retention member 862 can resiliently bend back to its previous position, thereby preventing the electrode 840a, 840b from sliding proximally or distally along the spine 814.

The proximal electrode retention region 860b includes a proximal retention member 862c and a distal retention member 862b. The proximal electrode retention region 860b need not be configured to permit the proximal electrodes 840b to pass over the distal retention member 862b. The distal electrode retention region 860a utilizes the central spine intersection 211 to prevent the distal electrodes 840a from moving distally once the distal electrodes 840a are in their respective final position.

Although the basket assembly 838 is shown as having two electrodes 840 disposed near each other on a given spine 814 and having alternating groupings of electrodes 840 on adjacent spines 814, the disclosed technology can include other configurations of electrodes 840 and spines 814 not shown. For example, the disclosed technology can include groupings of three or more electrodes 840 and/or multiple groupings of electrodes 840 disposed on spines 814. Thus, the disclosed technology is not limited to the particular configuration of electrodes 840 and spines 814 shown and described herein.

The effectiveness of applying pulsed field ablation (PFA), i.e., the ablation quality, may be estimated and/or rated based on computing a PFA ablation index (sometimes referred to herein as “PF index”, “AI”, “PFA AI” or simply “ablation index”) as described in U.S. Patent Publication No. 2025/0000573 filed Jun. 21, 2023 and published on Jan. 2, 2025, the entirety of which is incorporated herein by reference and attached in the appendix included with priority U.S. Provisional Patent Application No. 63/768,666, filed Mar. 7, 2025 (Attorney Docket No.: 253757.000534 (BIO6981USPSP1)). The PFA ablation index provides an indication of parameters of lesion created by PFA, e.g., the lesion area and/or depth. The lesion and thus the PFA ablation index may depend on the parameters of the applied pulses, including the contact force applied by the catheter on the heart wall which is an important parameter affecting the quality of the caused lesion, and the number of pulses, their frequency, duration, amplitude, and magnitude. In particular, unlike the number, frequency, duration and amplitude of the applied pulses which can be fixed in some examples, the applied force is controllable by the physician, thereby enabling the physician to control the quality of the lesion caused due to PFA and thus the PFA ablation index.

Optionally, the PFA ablation index may be more specifically dependent on contact force applied by one or more selected electrodes 40 of the medical probe 22. Optionally, the selected electrodes 40 are electrodes 40 that are determined to be touching the heart wall. The PFA ablation index may further depend on the specific location within the heart wall where the ablation is applied, and may be affected, for example, by the type of tissue, its thickness and its morphology.

Typically, ablation is performed by repeated applications of trains of pulses, with a pause, for example of about 1 second, between applications. The AI may be calculated and displayed after each application, by calculating and adding a Delta AI to the cumulative AI for all applications delivered, such that the user can decide whether and how to apply another train of pulses.

Typically, the pulse parameters are predetermined. Thus, the parameters that are typically controllable by the physician applying the ablation are the location and the force to be applied to the heart wall by the electrode. However, a physician may not have a good feel for how much contact force needs to be applied to obtain satisfactory PFA of the target area. Thus, by displaying the PFA AI predicted upon a currently applied force, the physician may fine tune the applied force to achieve the desired effect. Moreover, a physician may want to achieve a desired PFA AI and maintain that PFA AI over a lesion formed by multiple ablation sites. Thus, in some example embodiments, a computed and displayed predictive PFA AI may help the physician reach a desired PFA AI and/or achieve a uniform PFA AI over an ablation region formed by multiple ablation sites.

Thus, in accordance with some embodiments of the disclosure, contact force is monitored and a predicted PFA ablation index may be calculated based on the assumption that the physician will maintain that contact force throughout a predefined number of applications. The predicted PFA AI may be displayed to the physician and may provide indication to the physician on how much contact force to apply to achieve a desired PFA AI, e.g., a desired lesion size and/or depth. The predicted PFA displayed may be automatically and continuously updated as the contact force applied by the physician changes.

In some embodiments, the total force applied by the catheter on the heart wall may be measured, wherein the forces applied by one or more electrodes touching the heart wall may contribute to said force. In other embodiments, the separate forces applied by the one or more electrodes may be considered.

The index may be predicted using the same equation that is used to compute an actual PFA AI.

The index may be calculated by integrating the effect of each pulse over the predetermined number of pulses, where it is assumed that during all pulses the force is maintained at the same value as is currently measured.

Based on the prediction, the physician may then decide whether to ablate at the specific location, and whether to do so with the current force being applied on the tissue, or to change the location and/or the applied force.

The predicted PFA ablation index may be displayed to the user continuously or only when the catheter and/or electrodes contact the tissue, and the display may vary as the physician changes the location of the catheter and/or electrodes or the force applied by the catheter and/or the electrode against the heart wall.

The force applied by medical probe 22 on the heart wall, and optionally the location within the heart, may be used to calculate the PFA ablation index once ablation energy has been applied to the tissue. However, the force and optionally the location may also be used for predicting the PFA ablation index before the energy is applied assuming that medical professional 34 will apply the same amount of force, such that medical professional 34 may apply the required force at a specific location to achieve a desired lesion and/or a desired PFA AI. The predicted PFA ablation index may be calculated based on the force in the same manner as the actual PFA ablation index is calculated once the energy has been applied.

Control console 24 includes memory, processor unit with memory or storage with appropriate operating software stored therein, and user interface capability. Control console 24 may provide multiple functions, optionally including: (1) displaying on display 64 a current PFA index, calculated upon the force and location of medical probe 22, and a predicted PFA index, (2) modeling the endocardial anatomy in three-dimensions (3D) and rendering the model or anatomical map 62 for display on a display 64, (3) displaying on display 64 activation sequences (or other data) compiled from recorded electrograms in representative visual indicia or imagery superimposed on the rendered anatomical map 62, (4) displaying real-time location and orientation of multiple catheters within the heart chamber, and (5) displaying on display 64 sites of interest such as places where ablation energy has been applied.

In some embodiments, the model or anatomical map 62 may indicate thereon the PFA ablation index or a prediction thereof for a plurality of locations within the heart.

In some embodiments, the indices may be displayed for areas where the electrodes have applied force on the heart wall and indicate the PFA ablation index predicted upon the force applied at each such location.

In other embodiments, the indices may be displayed for all areas of the heart wall and indicate the predicted PFA ablation index calculated upon an average predetermined force, such that the physician can choose the best locations. In some embodiments, the average force used for prediction may be a parameter learned per physician, for example the force the physician usually applied during ablation.

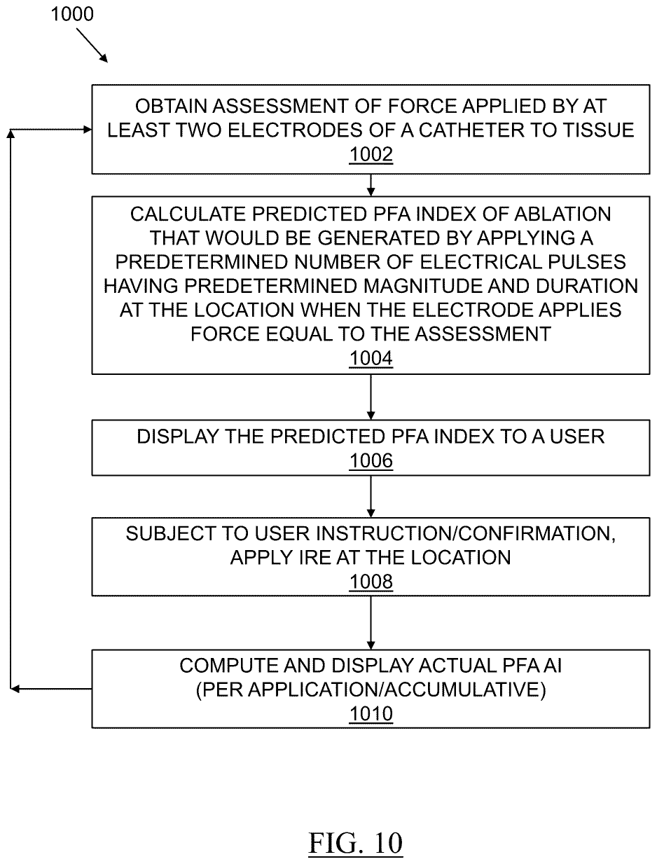

Referring now to FIG. 10, showing a flowchart of steps in a method 1000 for calculating, displaying and utilizing a predicted PFA index, in accordance with some exemplary embodiments of the disclosure.

The method 1000 can include obtaining 1002 a force assessment or measurement from a force sensor located at the distal end of the shaft and measuring the force at which the physician is pressing the catheter against a body tissue.

In some embodiments, when assessing the force, the local forces exerted by the electrodes of the electrode pairs that would be activated under the current protocol may be considered. For example, the sum of the forces exerted by the electrodes, the average, the maximum force applied by any of the electrodes or the like.

In other embodiments, the force may be calculated upon the sum, average maximum or any other measure of the forces applied by all electrodes. Optionally, the force is the force sensed on a distal end of the shaft proximal to the location at which the distal end assembly is connected to the shaft.

The method 1000 can further include calculating 1004 a predicted PFA ablation index which predicts the PFA ablation index that would be obtained by applying a predetermined number of electrical pulses or pulse trains having predetermined magnitude and predetermined duration at the location, when the electrode applies to the tissue amount of force equal to the assessed force.

The predicted PFA ablation index may be calculated using the same calculation that is used to compute the actual PFA AI once ablation has been applied. An important variable in this calculation is the force applied by the catheter on the heart wall, which the physician can affect. The calculation includes other parameters including the following: the number of pulses to be applied and their respective magnitude and duration, in accordance with the applicable protocol which can be predefined; information specific to the patient if available, or generic information based on the general structure of the heart otherwise; and the specific location within the heart and its characteristics, such as the thickness of the tissue at the relevant location, the tissue type, its conductivity, or the like.

The location information may be global to the catheter or specific to each electrode. In the latter case, the predicted PFA ablation index may be combined from the force applied to each active electrode and the characteristics of the specific location at which the electrode contacts the body tissue.

Calculating the Ablation Index via PFA is further described and shown in US Patent Application Publication US2021/0186604A1, the entirety of which is incorporated herein by reference as if set forth in full herein, a copy of which is attached in the Appendix included with priority U.S. Provisional Patent Application No. 63/768,666, filed Mar. 7, 2025 (Attorney Docket No.: 253757.000534 (BIO6981USPSP1)).

To predict and control ablation treatment, a PFA AI may be computed and displayed 1006 to a user. For PFA ablation, a possible scale can correspond to a size S indicating a depth of the lesion, which may depend on, F, P, and T, wherein Fis the force applied by the catheter to the tissue, P is the power dissipated during the ablation procedure; and Tis the time of the procedure, as derived from the number of repetitions of the pulses and train of pulses. In some embodiments, an estimate of size S may be given by finding an integral over time of an expression comprising functions of F, P, and T.

The estimate may be applied during PFA such that PFA can be halted when a desired lesion size and/or PFA AI is reached.

In an exemplary embodiment of the disclosure the PFA AI may be derived by calculating a summation over the time period of a product of the contact force and the number of repetitions.

The PFA Al may be calculated in accordance with the following formula:

$$PFAAI = {\sum }_{n = 1}^{N} [ {AI}_{n} - {AI}_{n \cdot 1} ]$$

wherein N equals the number of applications of PFA pulses (sometimes referred to as “PFA applications”), and has a predetermined maximum value. It is appreciated that each application of pulses adds to the total PFA AI.

AIₙ is an Ablation Index for the n^{th} application of pulses and is equal to the depth of the lesion multiplied by a factor A, with the depth being a logarithmic function of force for the particular application (force (n)) such that:

AIₙ=A*Bₙ*ln (force (n))+Cn; and

AI_{n·1}=A*Bₙ₋₁*ln (force (n))+C_{n·1}
wherein A is a number within a predetermined range,
Bₙ is a predefined parameter determined by the equation:

Bₙ=Bₒ*ln(n)+B1
where B₀ and B₁ are predefined constants, and
Cₙ is a predefined parameter determined by the equation:

Cₙ=Cₒ*exp(C₁*n)
where C₀ and C₁ are predefined constants.

The A, Bₙ and Cₙ values may be determined empirically for each application iteration and calibrated for each type of catheter and type of cardiac structure and given tissue characteristics.

It is noted that AIₙ and AIₙ₋₁ are calculated for each iteration such that the value of AIₙ₋₁ when used in the n^{th} iteration, is not taken from the previous calculation. The displayed PFA may also be updated accordingly with each application.

The predicted PFA AI may be similarly calculated. However, since it is assumed that the force applied over all applications is constant, the formula is simplified to:

Predicted PFA AI=AI_{N}−AI₁
Wherein
   AI₁=A*B₁+ln(force)+C₁
   AI_{N}=A+B_{N}*ln(force)+C_{N}
   force is the measured value of the force applied by the catheter at the prediction time and assumed to be fixed throughout all applications,

BN=B₀*ln(N)+B₁, and

CN=C₀*exp(C₁*N).

In some examples, both the PFA AI and the predicted PFA AI may be provided to the user as non-dimensional numbers or range of non-dimensional numbers between a minima and maxima of representing a less than ideal ablation and ideal lesion, respectively. In one example, the ablation index is a non-dimensional scale between two limits, such as 0 and 1000, 250 and 850, or the like.

It is noted that for the actual PFA, AIₙ and AIₙ₋₁ are calculated for each application such that the value of AIₙ₋₁ when used in the n^{th} iteration, is not taken from the previous calculation. The displayed PFA may also be updated accordingly with each application. The predicted AI, however, is computed for the predefined number of applications based on the instantaneous force as measured, and the predicted AI is the value displayed.

Based on the predicted ablation index the physician can decide for or against ablating at a specific location or repeating the ablation, and/or the physician can decide if more or less contact force is desired during ablation.

As previously described, the method 1000 can include displaying 1006 the predicted PFA ablation index to a user of the system, such as a physician. For example, the predicted PFA ablation index can be displayed on display 64. Optionally, the predicted PFA ablation index can be displayed alongside the display of an actual PFA ablation index.

Additionally or alternatively, a map of the heart may be updated wherein each of a plurality of locations on the map may be associated with and display predicted PFA indices that have been calculated when the physician brought the catheter to contact with the heart tissue. The map may be color coded, shade coded, pattern coded, or the like.

Additionally or alternatively, a map of the heart may be displayed which shows a generic map of predicted PFA ablation indices in multiple locations, based on the type of tissue and its thickness. The map may display the indices based on generic predetermined force or typical force applied by the physician, as learned from the current session or previous sessions of the physician.

The calculation and the display may be updated every predetermined period of time, such as every second, every 5 seconds, or the like. In alternative embodiments, the calculation and the display may be updated continuously, for example every time the catheter is moved.

Optionally subject to user's instruction, or subject to confirmation to a displayed notice, the generator may be operated by the user and IRE energy may be applied 1008 or supplied to the electrodes in accordance with the applicable protocol, and hence to the tissue. Thus, a predetermined number of pulses of electrical current of predetermined width are provided to the relevant electrodes and hence at the location, to create ablation.

The method 1000 can further include, subject to the ablation having been performed, computing 1010 the actual PFA ablation index may be and displaying the actual PFA ablation index. Typically, the actual PFA AI is computed on a per application basis and the cumulative PFA AI is displayed after each application as the ablation is being applied.

In some embodiments, following the application, the actual PFA ablation index may be compared to the predicted value, to obtain at least one difference.

In some embodiments, the comparison results, including the at least one difference, may be used for learning prediction parameters, such that future predictions are more accurate. For example, if the prediction is consistently higher or lower than the actual value, the prediction may be decreased or increased, respectively. In further cases, more complex relationships may be learned between any two or more of the applied force, tissue characteristics, the catheter structure and the applied protocol. The learning may be performed automatically by a processor, manually by a human operator, or in a hybrid manner.

It will be appreciated that the steps and modules disclosed above are in addition to the software, hardware, firmware or other modules required for operating the catheter, displaying the catheterization process, performing other calculations such as complex fractionated electrogram (CFE) analysis, generating the heart map, or the like. Further details for methods and systems may be found, for example in U.S. Pat. Nos. 8,676,305, 9,629,567, the entireties of which are incorporated herein by reference and attached in the Appendix included with priority U.S. Provisional Patent Application No. 63/768,666, filed Mar. 7, 2025 (Attorney Docket No.: 253757.000534 (BIO6981USPSP1)).

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions by, for example, the processor 46 and memory 66.

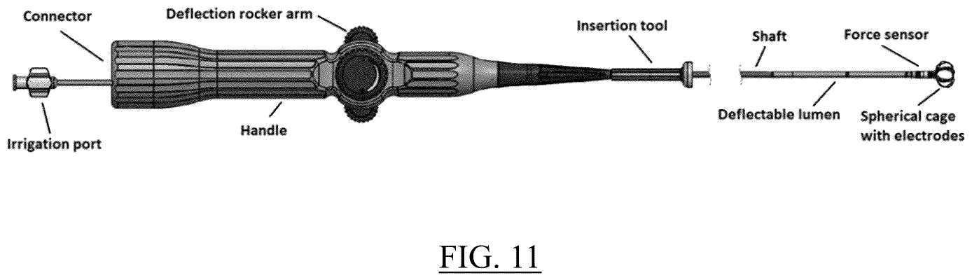

FIG. 11 is a schematic diagram of a catheter configured for mapping and ablation of tissue, in accordance with an example of the disclosed technology. The catheter shown in FIG. 11 can be the same or similar as the medical probe 22 shown and described previously herein. As shown, the disclosed technology can include a multi-electrode, contact force-sensing catheter with a distally located spherical cage that is 12 mm in diameter. The catheter can include a deflectable tip designed to facilitate electrophysiological mapping of the heart and to transmit pulsed field (PF) energy to 12 electrodes along the catheter cage for ablation purposes and can be used with, for example, a TRUPULSE™ Generator made by Biosense Webster Inc. of 31 Technology Drive, Suite 200, Irvine, CA 92618 USA.

The catheter shaft measures approximately 7.5 F with 8 F ring electrodes and can be deployed through a 10 F guiding sheath. The catheter can come assembled with an insertion tool located on the shaft to facilitate the collapse of the 12 mm diameter cage and subsequent insertion into the guiding sheath.

For ablation, the catheter is used in conjunction with the TRUPULSE™ Generator and is connected to the Connection Interface Unit (CIU) via the sterile interface cable. The generator can transmit biphasic, high voltage pulses to the electrodes on the catheter to perform ablation in bipolar mode. The catheter has magnetic-based navigation technology, allowing the CARTO® 3 System to display the location and orientation of the catheter. Additionally, the catheter can include force-sensing technology that provides a real-time measurement of contact force between the distal cage of the catheter and the heart wall.

Also shown in FIG. 11, starting from left to right in the figure, is an irrigation port, a connector (i.e., a location where one or more electrical wires can be connected), the handle, the deflection rocker arm (i.e., an actuator), the insertion tool (which can be used to help insert the basket catheter into the delivery sheath), the shaft, the deflectable lumen (i.e., the portion of the shaft configured to deflect radially outward from the longitudinal axis when actuated by the deflection rocker arm), the force sensor, and the spherical cage with electrodes (the basket assembly).

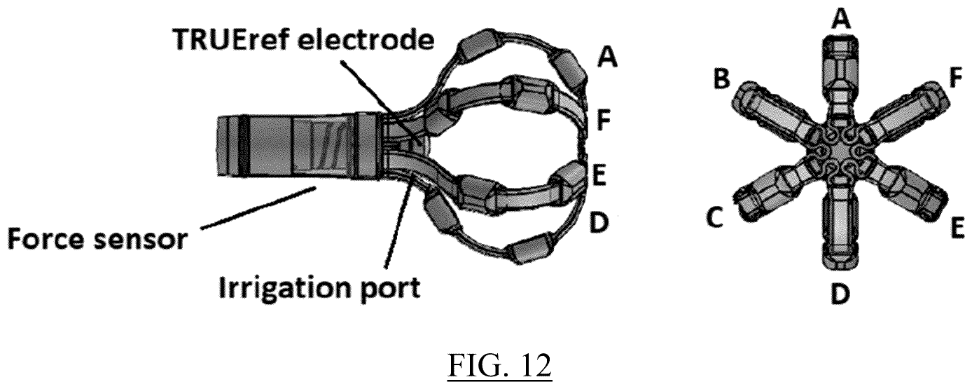

FIG. 12 is a schematic diagram of a basket assembly having a plurality of electrodes, in accordance with an example of the disclosed technology. As shown, the basket assembly (sometimes referred to herein as a “cage”, “spherical cage”, and/or “distal cage”) can include six spines (labeled as A-F) each having two electrodes disposed thereon as previously described. Furthermore, a references electrode (“TRUref electrode”) can be disposed in a space positioned between the plurality of spines, an irrigation port can be disposed near the reference electrode in the spaced positioned between the plurality of spines, and a force sensor can be disposed proximate the basket assembly.

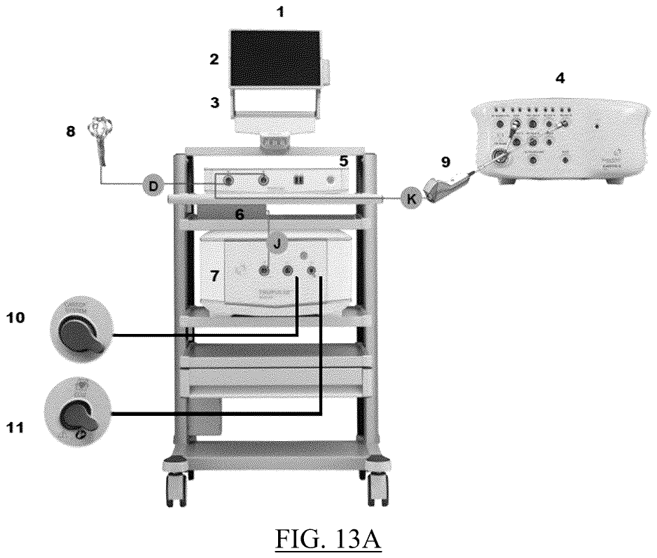

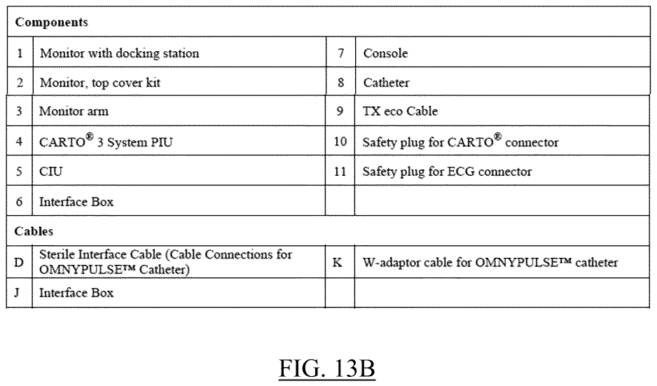

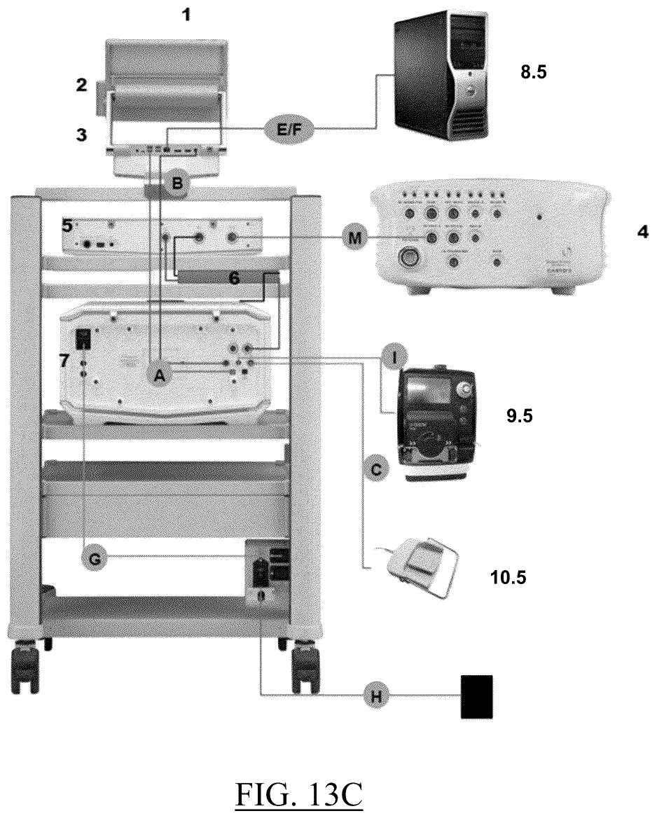

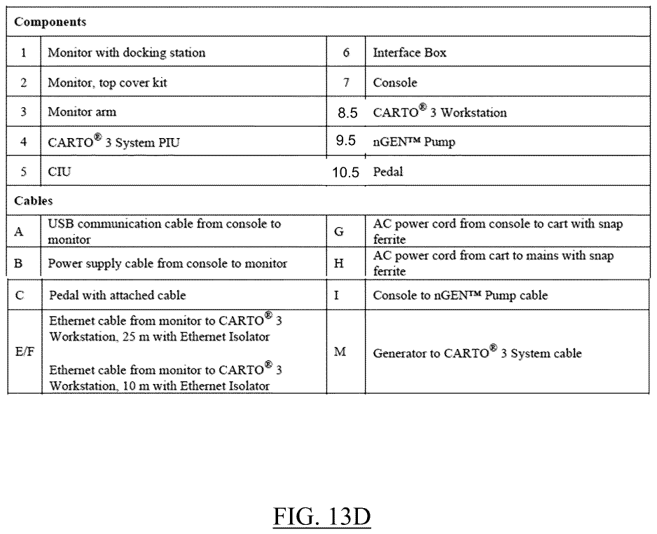

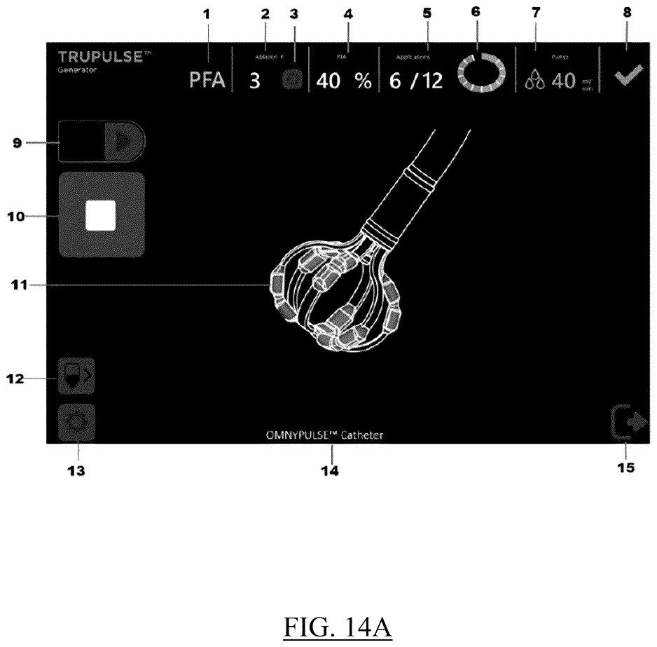

FIG. 13A is a schematic diagram of a front side of a system of mapping and ablating tissue while FIG. 13B is a table indicating components of the system shown in FIG. 13A, in accordance with an example of the disclosed technology. The details of the various components shown in FIG. 13A can be further seen in the table shown in FIG. 13B. For the sake of clarity, the system shown in FIG. 13A can be the same as or similar to the system shown and described in relation to FIG. 1. As shown, the system can include a monitor with docking station (1), a monitor and top cover kit (2), a monitor arm (3), a CARTO® 3 System patient interface unit (4), a catheter interface unit (5), an interface box (6), a console (7), a catheter (8), a TX eco Cable (9), a safety plug for CARTO® connector (10), and a safety plug for ECG connector (11). Further the system shown in FIG. 13A can include a sterile interface cable (cable connections for OMNYPULSE™ Catheter) (D), an interface box (J), and a W-adapter cable for OMNYPULSE™ catheter (K). The sterile interface cable (D) can provide an electronic interface between the OMNYPULSE™ Catheter and the TRUPULSE™ Generator. One end of the cable connects to the OMNYPULSE™ catheter, and the other end connects to the CIU via the TRUPULSE™ Generator W-adaptor cable for the OMNYPULSE™ catheter.

FIG. 13C is a schematic diagram of a rear view of the system shown in FIG. 13A while FIG. 13D is a table indicating components of the system shown in FIG. 13C, in accordance with an example of the disclosed technology. As shown, the disclosed technology can include a monitor with docking station (1), a monitor and top cover kit (2), a monitor arm (3), a CARTO® 3 System patient interface unit (4), a catheter interface unit (5), an interface box (6), a console (7), a CARTO3® workstation (8.5), nGEN™ Pump (9.5), and a pedal (10.5). Further the system shown in FIG. 13C can include a USB communication cable from the console to the monitor (A), a power supply cable from the console to the monitor (B), cable between the pedal to the console (c), an ethernet cable from the monitor to the CARTO® 3 workstation (E) (25m with ethernet isolator) and an ethernet cable from the monitor to the CARTO® 3 workstation (F) (10m with ethernet isolator), an AC power cord from the console to the cart with snap ferrite (G), an AC power cord from the cart to mains power with snap ferrite (H), a console to nGEN™ Pump cable (I), and a cable from the generator to the CARTO® 3 system. The Tx eco Cable (9) can communicate force and location data from the OMNYPULSE™ Catheter to the Patient Interface Unit (PIU) of the CARTO® 3 System.

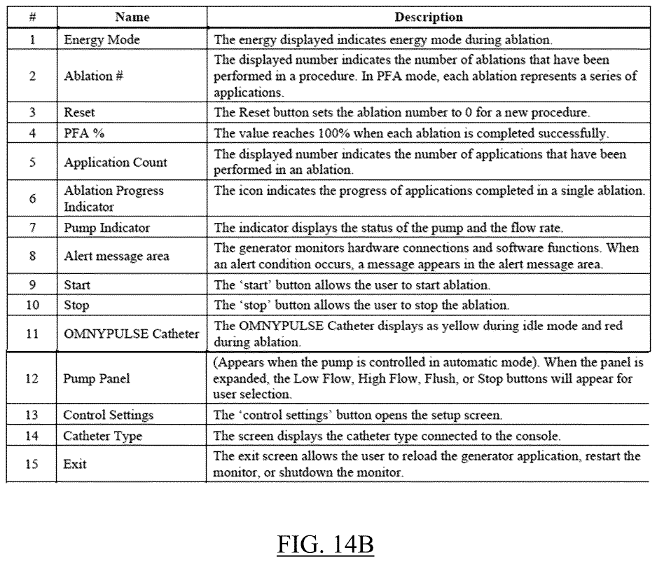

FIG. 14A is an illustration of a user interface of a system for mapping and ablating tissue while FIG. 14B is a table indicating features of the user interface shown in FIG. 14A, in accordance with an example of the disclosed technology. The illustrated user interface, for example, can be displayed on the display 64 shown in FIG. 1. The user interface can include features such as an indicator showing the energy mode (1) which can be indicative of the type of energy applied during application, an ablation number (2) which can indicate the number of ablations that have been performed in a procedure (in PFA mode, each ablation represents a series of applications), a reset button (3) that can reset the ablation number to 0 for a new procedure, a PFA % indicator (4) which reaches 100% when each ablation is completed successfully, an application count (5) which can indicate the number of applications that have been performed in an ablation procedure, an ablation progress indicator (6) that can indicate the progress of applications completed in a single ablation, a pump indicator (7) that can display the status of the irrigation pump and the flow rate of the fluid, an alert message area (8) that can indicate errors or warnings associated with hardware and software functions, a start button (9) that allows a user to start ablation, a stop button (10) that allows a user to stop ablation, an OMNYPULSE catheter indicator (11) that can display as various colors indicative of a status (e.g., yellow for an idle mode and red for ablation), a pump panel (12) that can appear when the pump is controlled in automatic mode and can be expanded to display a low flow, high flow, flush, or stop buttons for user selection, a control settings icon (13) that can open a setup screen, a catheter type icon (14) that can display the catheter type connected to the console, and an exit icon (15) that allows a user to reload the generator application, restart the monitor, or shutdown the monitor. It will be appreciated, however, that the features shown and described in FIGS. 14A and 14B are offered for illustrative purposes and the disclosed technology is not limited to the features and functions shown and described herein.

The start button can include a drag feature. After the start button is dragged or the pedal is pressed, there is a countdown until ablation begins. Each ablation (i.e., multiple applications of PF energy) will deliver anywhere up to 12 applications (series of pulses) per targeted site. When the stop button is pressed or the pedal is released, the delivery of energy is terminated.

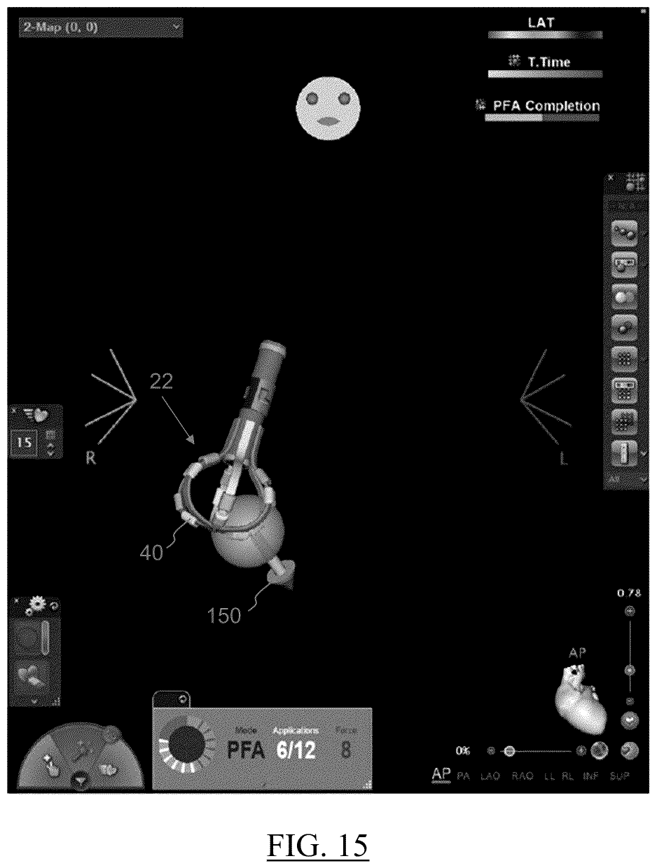

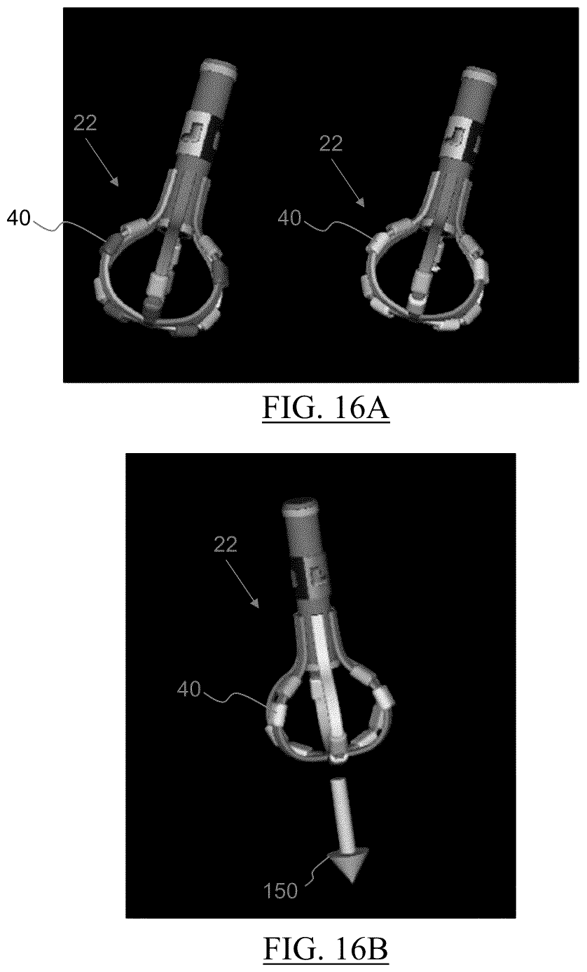

FIG. 15 is an illustration of another user interface used with a system for mapping and ablation tissue, in accordance with an example of the disclosed technology. The user interface shown in FIG. 15 can be an alternative view of the medical probe 22 as displayed on the display 64 to help a physician visualize placement and operation of the medical probe 22. As shown further in FIGS. 16A and 16B, the medical probe 22 can be displayed with colored electrodes 40 that can be indicative of their current function (e.g., yellow=no ablation being applied, purple=ablation being applied). Further, an arrow 150 can be displayed on or near the medical probe 22 indicative of the force angle (relative to the longitudinal axis 86) from the center of the medical probe 22. In some examples, the arrow 150 can be scaled larger as the force increases and scaled smaller as the force decreases.

FIGS. 16A and 16B illustrate various views of an illustration of the medical probe as displayed on a user interface, in accordance with an example of the disclosed technology. The views shown in FIGS. 16A and 16B can be displayed, for example, on the display 64 illustrated in FIG. 1. In some examples, as shown in FIG. 16A, the display 64 can be configured to show the electrodes the medical probe 22 in a first color (e.g., purple) when ablative energy is being applied through the electrodes to the tissue and in a second color (e.g., yellow) when no ablative energy is being applied through the electrodes to the tissue. Furthermore, as shown in FIG. 16B an arrow 150 can be displayed on or near the medical probe 22 indicative of the force angle (relative to the longitudinal axis 86) from the center of the medical probe 22. In some examples, the arrow 150 can be scaled larger as the force increases and scaled smaller as the force decreases.

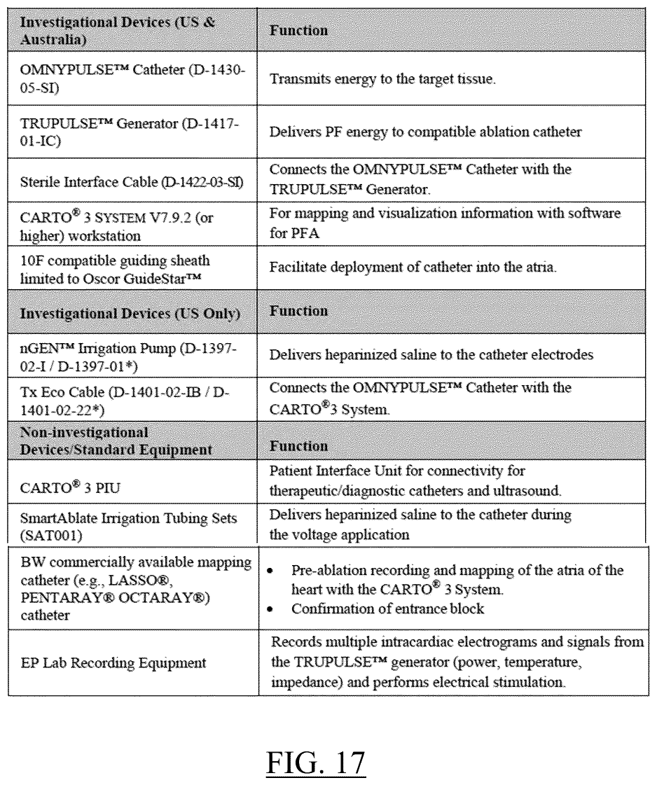

FIG. 17 is a table indicating various components that can be used with a system for mapping and ablation tissue, in accordance with an example of the disclosed technology. As shown, the system can include: an OMNYPULSE™ Catheter (D-1405-05-SI) that can be configured to transmit ablative energy to the target tissue; a TRUPULSE™ Generator (D-1417-IC) that can deliver pulsed field energy to the compatible ablation catheter (e.g., the OMNYPULSE™ Catheter, the medical probe 22); a Sterile Interface Cable (D-1422-03-SI) that connects the OMNYPULSE™ Catheter with the TRUPULSE™ Generator; a CARTO® 3 SYSTEM V7.9.2 (or higher) workstation for mapping and visualization information with software for PFA; a 10 F compatible guiding sheath limited to Oscor GuideStar™ that can facilitate deployment of catheter into the atria; an nGEN™ Irrigation Pump (D-1397-02-I/D-1397-01) that can deliver heparinized saline to the catheter electrodes; a Tx Eco Cable (D-1401-02-IB/D-1401-02-22) that can connect the OMNYPULSE™ Catheter with the CARTO®3 System; a CARTOR 3 PIU that can be a Patient Interface Unit for connectivity for therapeutic/diagnostic catheters and ultrasound; a SmartAblate Irrigation Tubing Sets (SAT001) that can deliver heparinized saline to the catheter during the voltage application; a BW commercially available mapping catheter (e.g., LASSO®, PENTARAY® OCTARAY®) catheter can be configured for pre-ablation recording and mapping of the atria of the heart with the CARTO® 3 System and for confirmation of entrance block; and EP Lab Recording Equipment that can be configured to record multiple intracardiac electrograms and signals from the TRUPULSE™ generator (power, temperature, impedance) and performs electrical stimulation.

The foregoing devices are offered for illustrative examples and should not be construed as limiting the scope of the system disclosed and described herein. Rather, the devices described above are illustrative of one particular configuration that has been used for conducting studies illustrating the efficacy of the disclosed technology as further described below.

### Example Studies

The disclosed technology can be further understood according to the following studies. As will become apparent, the catheter described herein and shown in the drawings is an effective device, system, and method of ablating tissue to prevent aberrant electrical signals in a heart. The studies described herein are offered for illustrative purposes and should not be construed as limiting. That is, the studies and associated results herein are offered to help illustrate the efficacy of the disclosed technology and should not be construed as describing the limits of the disclosed technology.

### Porcine Model Study

An initial in-vivo animal study titled “Safety Evaluation of Biosense Webster OMNYPULSE™ PFA Ablation Catheter and TRUPULSE™ Generator in a Chronic (GLP) Swine Beating Heart Model” performed under a predetermined protocol was GLP compliant and conformed to the guidelines for nonclinical laboratory studies as described in the Code of Federal Regulations, 21 Part 58. The purpose of this study was to demonstrate the overall safety of the OMNYPULSE™ PFA Platform, when ablating at clinically relevant anatomical locations like the pulmonary veins and left posterior wall, in a simulated clinical use scenario in combination with a CARTO® 3 System, nGEN irrigation pump, catheter interface cables, communication interface box and a pacing system in a chronic beating heart model. Various application doses of PFA were delivered to characterize performance and safety at clinically recommended settings and upper bound of settings.

The study consisted of two arms with 6 animals in each group: (1) Nominal Dose (6 applications per ablation) and (2) 2× Nominal Dose (12 applications per ablation). A total of 12 animals (plus 2 backups) were included in the study.

Safety of the Test devices were evaluated using the following assessments: occurrence of thrombus and/or char on the test catheter, following energy delivery; occurrence of steam pop leading to pericardial effusion and/or cardiac tamponade; occurrence of mural thrombus in the treated heart chambers; occurrence of pulmonary vein (PV) stenosis (defined as >70% reduction in PV ostial diameter); occurrence of collateral damage to surrounding organs (aorta, lungs, esophagus, phrenic nerves, bronchi, pericardium); occurrence of intracardiac tissue damage due to mechanical injury from the device; and occurrence of peripheral thrombo-emboli in upstream/downstream organs. Efficacy of the Test devices were evaluated using the following assessments: ability to demonstrate 100% acute effectiveness of ablation in both right and left atrium; and ability to demonstrate 70% chronic effectiveness of ablation in both right and left atrium.

Testing was performed on thirteen (13) male Yorkshire pigs. One porcine subject was removed and replaced from the initial cohort due to animal movement resulting in an errant ablation and premature death. The cause was not related to the investigational device or system. Histopathological evaluation of this porcine animal confirmed cause of premature death as non-intended ablation near the AV node. All animals were treated using PFA with the TRUPULSE™ Generator and OMNYPULSE™ Catheter. After concluding Day 28±2 follow-up procedures, animals were humanely euthanized under anesthesia, and gross necropsies were performed.

Energy was applied to targeted tissue areas wall thickness and their anatomic relevance to an atrial fibrillation treatment procedure. These included areas in the left atrium (LA) such as 2 pulmonary veins (at minimum), roof/posterior wall, and mitral isthmus, and in the right atrium (RA) the CTI and posterior wall.

Throughout the procedure, no signs of respiratory distress, pain or other abnormal behaviors were observed in any of the study animals. Additionally, no mural thrombus, coagulum or charring, cardiac effusion and/or tamponade were noted for the duration of the study nor was evidence of steam pop detected in any of the enrolled animals. All 12 test animals survived until their scheduled termination time points.

Ablation energy was applied directly to the phrenic nerve. Subsequent phrenic nerve capture after pacing was successful in all animals in both arms, demonstrating no loss of phrenic function. Pulmonary vein stenosis assessment was based on fluoroscopic measurements. No clinically relevant reduction in PV diameter was noted for any animal following Day 1 ablations and Day 28±2 follow-up (remap) procedures. Upon gross pathological inspection, it was noted that ablations were created in intended anatomical regions with no significant collateral injury.

All animals in both treatment arms showed a similar safety profile for the specified protocol endpoints.

All safety acceptance criteria for the study were met.

Three physicians performed the ablation treatment on each of 1 animal per arm.

Confirmation of elimination of PV potential using a Pentaray® diagnostic mapping catheter, following initial ablation procedure was confirmed in all animals, meeting the expectation of 100% acute effectiveness of ablation in the left atrium.

For chronic evaluations on Day 28±2, all PFA treated animals presented 100% chronic PV isolation.

All effectiveness acceptance criteria for the study were met.

There were no gross signs of collateral injury to any of the adjacent anatomic structures, including aorta, lungs, esophagus, bronchi, pericardium, and/or phrenic nerve adjacent to the ablation lesions. All histology findings were within expectations.

As part of the study, average contact force for each ablation along with number of applications was used retrospectively to derive the PF Index. The retrospective analysis yielded PF index values representative of the PF index values of 400 and 550 used in a clinical scenario while confirming the safety and efficacy endpoints were met from the GLP study.

The OMNYPULSE™ PFA Platform meets all acceptance criteria as listed in protocol P/TR-0039030.

This study confirms the overall effectiveness and safety of the OMNYPULSE™ PFA Platform, when simulating a clinical pulmonary vein isolation (PVI) procedure in a porcine beating heart model.

### Clinical Study

To further test out the efficacy of the technology disclosed herein, including the medical probe shown and described throughout, a study was conducted on a plurality of patients. The study was a clinical investigation involving an interventional, prospective, single-arm, multicenter, non-randomized, premarket clinical evaluation of the BWI OMNYPULSE™ PFA Platform to demonstrate safety and long-term effectiveness of the system for the treatment of Paroxysmal Atrial Fibrillation (PAF) comparing to corresponding performance goals. Eligible patients were 18 to 75 years of age, were diagnosed with symptomatic paroxysmal AF, and had been selected for AF ablation by PV isolation (PVI).

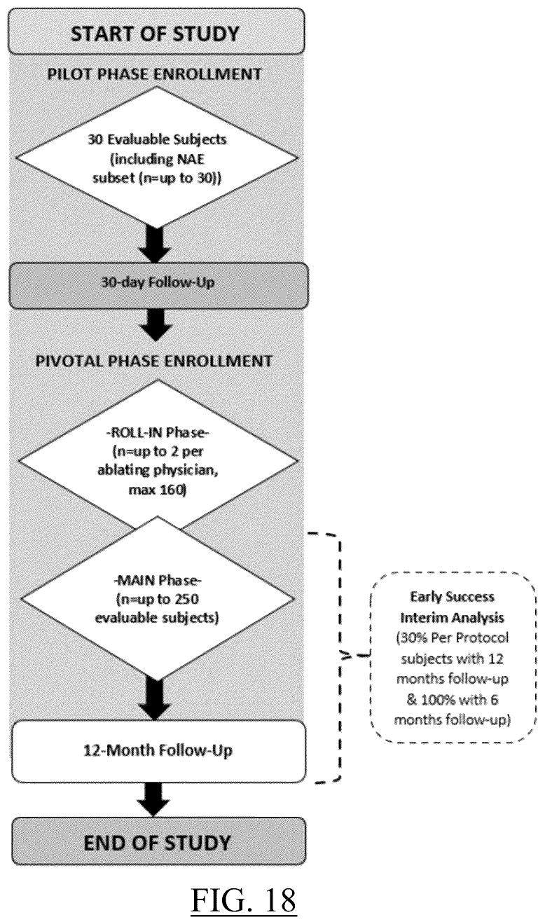

FIG. 18 is a chart illustrating various phases of a study of a catheter configured for mapping and ablating tissue, in accordance with an example of the disclosed technology. The effectiveness and safety endpoints were compared to predetermined performance goals. As shown, an interim review of safety data was performed on 30 subjects during a pilot phase with a 30-day follow-up to ensure the initial safety and effectiveness goals were met. 188 subjects were ultimately enrolled in the Pivotal Main Phase following the enrollment requirements set forth in the protocol as shown in FIG. 18.

The Pilot Phase included a neurological assessment evaluation (NAE) subset for a focused neurological evaluation. NAE subjects were assessed for incidences of symptomatic and asymptomatic pre- and post-ablation cerebral emboli.

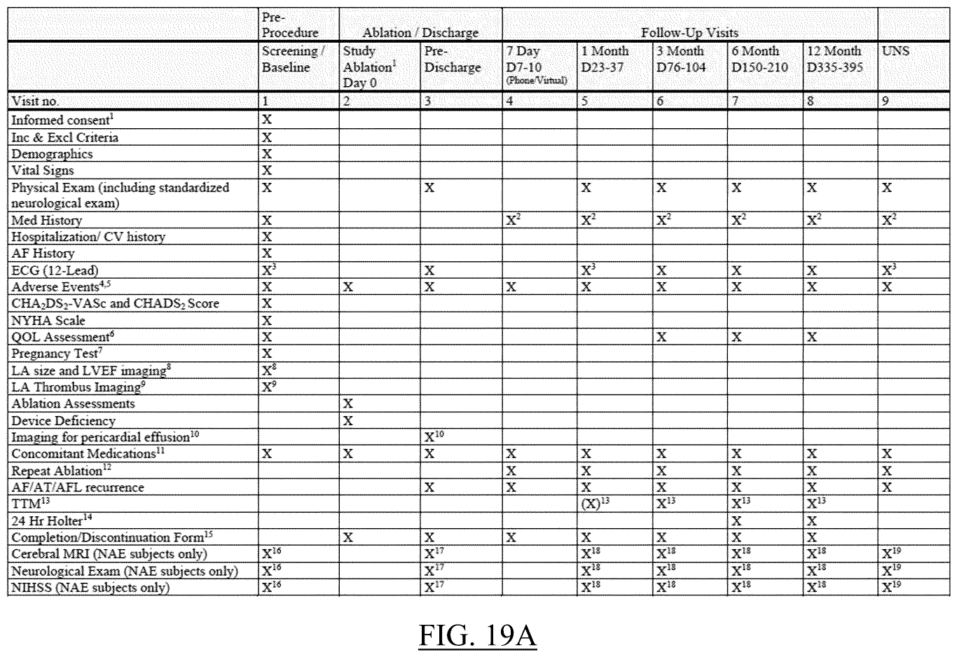

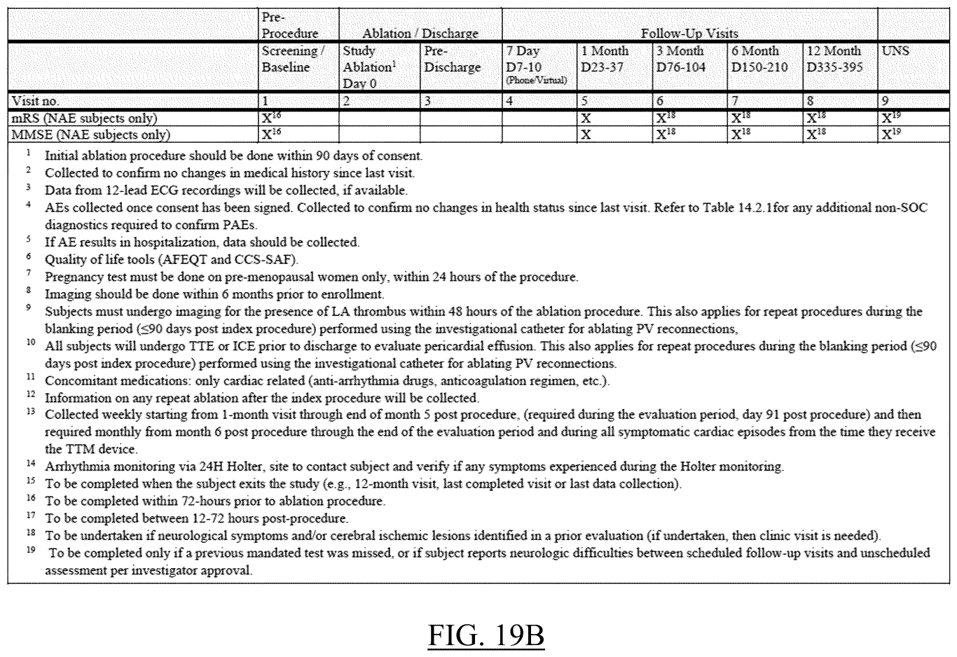

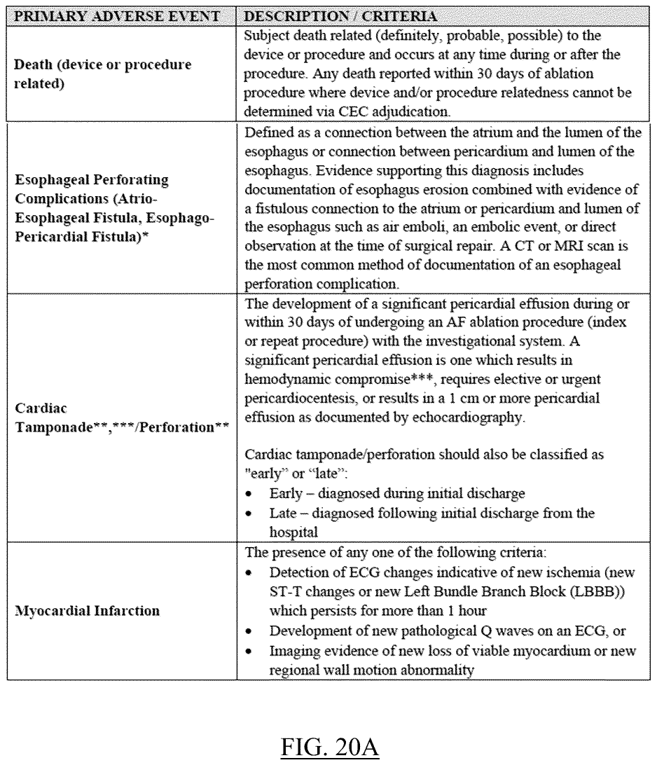

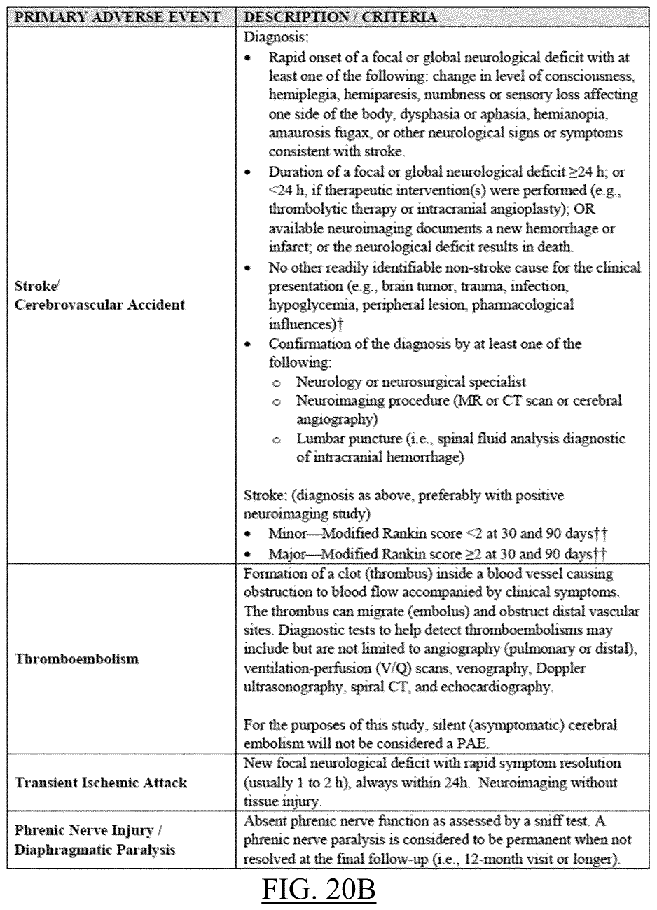

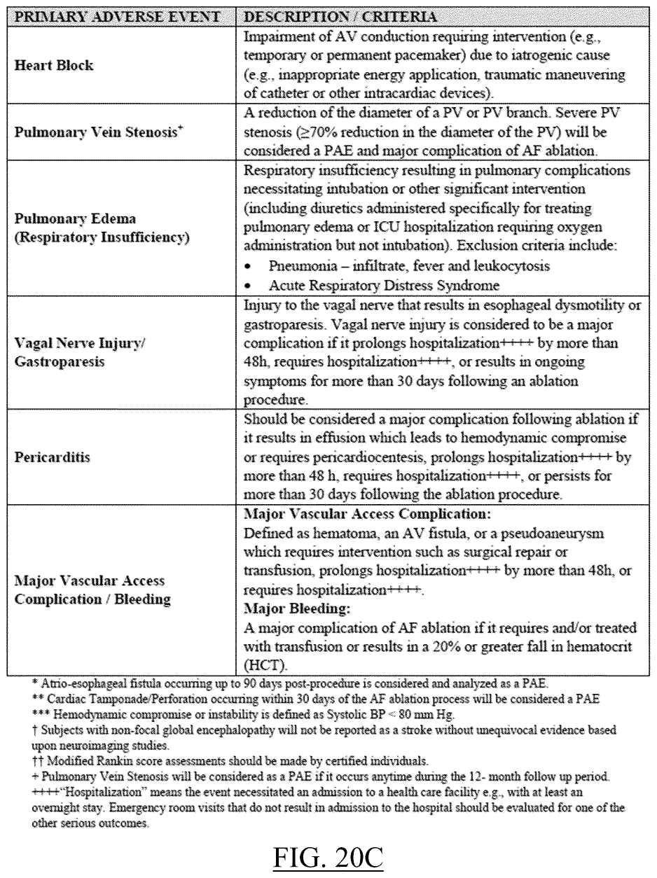

FIGS. 19A and 19B are a table illustrating a schedule of events of the study, in accordance with an example of the disclosed technology. The table illustrates the various events that are to occur during the study from the initial screening which happens before procedure all the way to 12-months following the procedure. For the sake of simplicity, the various events will not be described herein but details of the events, including various screening and investigational steps, are illustrated on the table shown in FIGS. 19A and 19B (the table from FIG. 19A being continued on FIG. 19B). FIGS. 20A, 20B, and 20C are a table of primary adverse events, in accordance with an example of the disclosed technology. As shown, the primary adverse events identified and tracked during the study included: death (device or procedure related), esophageal perforating complications (atrio-esophageal fistula, esophago-pericardial fistula), cardiac tamponade/perforation, myocardial infarction, stroke/cerebrovascular accident, thromboembolism, transient ischemic attack, phrenic nerve injury/diaphragmatic paralysis, heart block, pulmonary vein stenosis, pulmonary edema (respiratory insufficiency), vagal nerve injury/gastroparesis, pericarditis, and major vascular access complication/bleeding. Additional details for each of the above-listed primary adverse events can be found in FIGS. 20A, 20B, and 20C.

The primary objective of this study was to demonstrate the safety and 12-month effectiveness of the OMNYPULSE™ PFA Platform for pulmonary vein isolation (PVI) in the treatment of subjects with symptomatic paroxysmal atrial fibrillation (PAF). The secondary objective of this study was to assess the improvement of quality of life in study subjects, and to evaluate the safety and 12-month effectiveness of a zero/low fluoroscopy workflow when using OMNYPULSE™ PFA Platform for pulmonary vein isolation (PVI) in the treatment of subjects with symptomatic paroxysmal atrial fibrillation (PAF).

The primary safety endpoint of the study is the occurrence of early onset PAEs (that subjects experience within seven (7) days of an ablation procedure, unless otherwise indicated) which uses the OMNYPULSE™ PFA Platform per protocol, including the initial and repeat procedures. PAEs include the following adverse events:

| | |
|---|---|
| Esophageal perforating complications | Phrenic Nerve Paralysis |
| (Atrio-Esophageal Fistula, Esophago- | (permanent) |
| Pericardial Fistula) | |
| Cardiac Tamponade/perforation | Pulmonary Vein Stenosis |
| Death (device or procedure related) | Stroke/CVA |
| Major Vascular Access | Thromboembolism |
| Complication/Bleeding | |
| Myocardial Infarction (MI) | Transient Ischemic Attack (TIA) |
| Pericarditis | Pulmonary Edema (Respiratory |
| | Insufficiency) |
| Heart Block | Vagal Nerve Injury/Gastroparesis |

The primary effectiveness endpoint of the study was time to occurrence of documented (symptomatic and asymptomatic) atrial tachyarrhythmia (AF, AT, or AFL of unknown origin+) episodes based on electrocardiogramds on sponsor provided TTM or Holter device or continuous on a standard 12-lead ECG) during the effectiveness evaluation period (Day 91-Day 365 post index procedure) and any of the following failure modes:
Acute procedural failure, including:
   Failure to confirm entrance block in all pulmonary veins at the end of the procedure. (Note: Subjects who have the study catheter inserted but do not undergo ablation via the study catheter due to study device-related (OMNYPULSE™ catheter, TRUPULSE™ generator, or CARTO®) reasons will be considered as acute effectiveness failures; subjects who are discontinued due to non-study device-related reasons (e.g., other equipment or anatomy that precludes treatment with the OMNYPULSE™ catheter or a commercially available catheter) will not be considered as acute effectiveness failures.)
Repeat ablation failure, including:
   More than 1 repeat ablation procedures for AF/AT/AFL of unknown origin+ during the 3-month Blanking Period (Day 0-90 post index procedure).
   Any repeat ablation procedure for AF/AT/AFL of unknown origin+ during the evaluation period.
Surgical treatment for AF/AT/AFL (of unknown origin+) after the index ablation procedure.
Non-study catheter failure, including:
   Use of a non-study catheter (NSC) to treat pulmonary vein targets to achieve isolation of PVs or to ablate left atrial non-PV AF targets during the index procedure.
   Use of a non-study catheter to ablate pulmonary vein targets due to PV reconnection during a repeat ablation in the blanking period.
AAD failure:
   Taking a new AAD (Class I & III) for atrial tachyarrhythmia (AF, AT or AFL of unknown origin+) or taking a previously failed Class I/III AAD at a greater than the highest ineffective historical dose for AF/AT/AFL of unknown origin+ at any time beyond the 3-month follow-up visit window (i.e., at any time from day 105 to 365 post index procedure)
   Taking amiodarone at a dose greater than the historical highest ineffective maintenance dose at any time post index procedure.
Any DC cardioversion procedure during the evaluation period for documented atrial tachyarrhythmia (AF, AT or AFL of unknown origin+) recurrences ascertained through protocol specified arrhythmia monitoring methods (12 lead ECG, TTM and Holter).

The study included a secondary endpoint of determining a quality of life improvement (AFEQT): Atrial Fibrillation Effect on Quality-of-Life (AFEQT) Questionnaire will be used to assess the quality-of-life post ablation procedure. The endpoint is the change in quality of life, as measured by the total AFEQT score, at 12-month post procedure, in comparison to baseline assessment.

Furthermore, the study included the following additional safety endpoints:
Occurrence of Unanticipated (Serious) Adverse Device Effects (UADEs and USADEs)
Occurrence of non-primary SAEs within 7 days (early onset), >7 to 30 days (peri-procedural) and >30 days (late onset) of initial ablation
Occurrence of non-serious AEs within 7 days (early onset), >7 to 30 days (peri-procedural) and >30 days (late onset) of initial ablation
Occurrence of bleeding complication (ISTH definitions): a) major bleeding, b) clinically relevant non-major bleeding and c) minor bleeding.

The study further included the following additional effectiveness endpoints:
Acute procedural success: the electrical isolation of all PVs which is evidenced by the confirmation of entrance block at the end of the procedure.
Acute reconnection: The reconnection of one or more PVs after adenosine/isoproterenol challenge.
First pass isolation: the achievement of entrance block evaluated prior to the adenosine/isoproterenol challenge.
Repeat ablation procedures: The occurrence of repeat ablation procedures for left atrial arrythmia (AF, AT, or AFL of unknown origin+) within the 12-month follow-up period.
12-Month Effectiveness Success Off Class I/III AAD: Freedom from documented (symptomatic and asymptomatic) AF/AT/AFL (of unknown origin+) episodes based on electrocardiographic data (≥30 seconds on sponsor provided TTM or Holter device or continuous on a standard 12-lead ECG) during the effectiveness evaluation period (Day 91-Day 365) without:
   Taking any AAD (Class I or III) for AF/AT/AFL (of unknown origin+) at any time beyond the 3-month follow-up visit window (i.e., at any time from day 105 to 365 post index procedure).
   Taking amiodarone at a dose greater than the historical highest ineffective maintenance dose at any time post index procedure.
12-Month Single Procedure Treatment Success with additional failure modes: The 12-month single procedure success is defined as freedom from documented symptomatic and asymptomatic AF/AT/AFL (of unknown origin+) episodes based on electrocardiogramds on sponsor provided TTM or Holter device or continuous on a standard 12-lead ECG) through the effectiveness evaluation period following a single index ablation procedure. Subjects that undergo repeat procedure(s) will be deemed failures for this endpoint. Additionally, subjects with any of the following failure modes will also be deemed failures of this endpoint:
   Acute procedural failure
   Non-study catheter failure, including:
      use of a non-study catheter (NSC) to treat pulmonary vein targets/isolation of PVs and left atrial non-PV AF targets during the index procedure.
   AAD failure:
      Taking a new AAD (Class I/III) for atrial tachyarrhythmia (AF, AT or AFL of unknown origin+) or taking a previously failed Class I/III AAD, at a greater than the highest ineffective historical dose, for AF/AT/AFL (of unknown origin+) at any time beyond the 3-month follow-up visit window (i.e., at any time from day 105 to 365 post index procedure)
      Taking amiodarone at a dose greater than the historical highest ineffective maintenance dose at any time post index procedure.
   Any DC cardioversion procedure during the evaluation period for documented atrial tachyarrhythmia (AF, AT or AFL of unknown origin+) recurrences ascertained through protocol specified arrhythmia monitoring methods (12-lead ECG, TTM and Holter).
12-Month Clinical Success: Freedom from documented symptomatic AF/AT/AFL (of unknown origin+) episodes based on electrocardiographic data (>30 seconds on sponsor provided TTM or Holter device or continuous on a standard 12-lead ECG) during the effectiveness evaluation period (Day 91-Day 365).
QOL Improvement (CCS-SAF): The change of scores of Class 0 to Class 4 at baseline and 12M post procedure.
Hospitalization for Cardiovascular Events: The change in occurrence of hospitalization for cardiovascular events from 12M prior to the index procedure and during the 12M follow-up post index procedure.
DC Cardioversion: The change in occurrence of DCCV for AF/AT/AFL (of unknown origin+) from 12M prior to the index procedure and during the 12M follow-up post index procedure.
AAD Utilization: Taking AAD for the treatment of AF/AT/AFL (of unknown origin+) during the 12M follow-up period.

To be included in the study, the participants had to meet all of the following criteria:
1. Symptomatic paroxysmal AF with:
   a. At least two (2) symptomatic AF episodes within last six (6) months from enrollment,
   b. At least one (1) AF episode electrocardiographically documented within 12 months prior to enrollment. Electrocardiographic documentation may include, but is not limited to electrocardiogram (ECG); Transtelephonic monitoring (TTM), Holter monitor or telemetry strip, and
   c. A physician's note indicating that the recurrent AF is self-terminating within 7 days.
2. Failed at least one (1) AAD (Class I or Class III) as evidenced by recurrent symptomatic AF, intolerable side effects to the AAD, or contraindication to the AAD.
3. Age 18-80 years.
4. Willing and capable of providing consent.
5. Able and willing to comply with all pre-, post- and follow-up testing and requirements.

Any candidates meeting the following criteria were excluded from the study:
1. Previously diagnosed with persistent AF (>7 days in duration)
2. AF secondary to electrolyte imbalance, thyroid disease, or reversible or non-cardiac cause (e.g., untreated documented obstructive sleep apnea and acute alcohol toxicity).
3. Previous surgical or catheter ablation for AF
4. Patients known to require ablation outside the PV ostia and outside the CTI region (e.g., atrioventricular reentrant tachycardia, atrioventricular nodal reentry tachycardia, atrial tachycardia, ventricular tachycardia and Wolff-Parkinson White)
5. Documented severe dilatation of the LA (LAD >50 mm) antero-posterior diameter on imaging within 6 months prior to enrollment.
6. Documented LA thrombus by imaging within 48 hours of the procedure.
7. Documented severely compromised LVEF (LVEF <40%) by imaging within 6 months prior to enrollment.
8. Uncontrolled heart failure or New York Heart Association (NYHA) Class III or IV
9. History of blood clotting, bleeding abnormalities or contraindication to anticoagulation (heparin, warfarin, or dabigatran),
10. Documented thromboembolic event (including TIA) within the past 6 months.
11. Previous PCI/MI within the past 2 months
12. Coronary Artery Bypass Grafting (CABG) surgery within the past 6 months (180 days)
13. Valvular cardiac surgical/percutaneous procedure (i.e., ventriculotomy, atriotomy, valve repair or replacement and presence of a prosthetic valve).
14. Unstable angina within 6 months
15. Anticipated cardiac transplantation, cardiac surgery, or other major surgery within the next 12 months.
16. Significant pulmonary disease (e.g., restrictive pulmonary disease, constrictive or chronic obstructive pulmonary disease) or any other disease or malfunction of the lungs or respiratory system that produces severe chronic symptoms.
17. Significant congenital anomaly or medical problem that in the opinion of the investigator would preclude enrollment in this study.
18. Prior diagnosis of pulmonary vein stenosis
19. Pre-existing hemi diaphragmatic paralysis
20. Acute illness, active systemic infection, or sepsis
21. Presence of intracardiac thrombus, myxoma, tumor, interatrial baffle or patch or other abnormality that precludes catheter introduction or manipulation.
22. Severe mitral regurgitation (Regurgitant volume ≥60 mL/beat, Regurgitant fraction ≥50%, and/or Effective regurgitant orifice area ≥0.40 cm2)
23. Presence of an implanted pacemaker or Implantable Cardioverter-Defibrillator (ICD) or other implanted metal cardiac device (other than coronary stents) that may interfere with the PF energy field.
24. Presence of a condition that precludes vascular access (such as IVC filter)
25. Current enrollment in an investigational study evaluating another device or drug.
26. Women who are pregnant (as evidenced by pregnancy test if pre-menopausal), lactating, or who are of child-bearing age and plan on becoming pregnant during the course of the clinical investigation.
27. Life expectancy less than 12 months
28. Presenting contra-indications for the devices used in the study, as indicated in the respective Instructions for Use (IFU).
29. Contraindication to use of contrast agents for MRI such as advanced renal disease, etc. (at PI discretion).
30. Presence of iron-containing metal fragments in the body.
31. Unresolved pre-existing neurological deficit.

Pre-procedure assessments were performed within 90 days prior to the index AF ablation procedure. The ablation procedure was performed within 90 days of signing informed consent by the subject.

Assessments with a shorter window prior to the AF ablation procedure are noted below:
Demographics (age, gender, etc.)
Medical History: including but not limited to arrhythmia, heart disease, thromboembolic events, lung/respiratory problems.
AF History (first evidence of AF, number of episodes, symptoms, etc.).
NYHA Functional Class Scale (for subjects with congestive heart failure).
CHA2DS2 VASc and CHADS2 Score: was used to assess the risk of stroke.
Medication History: Medication history (cardiac medication, AAD medication, anticoagulation regimen and any other clinically significant medication history) was gathered by interview or from medical records.
Anticoagulation Therapy: Uninterrupted anticoagulation management for at least 3 weeks prior to ablation procedure was mandatory for each study subject. For subjects on warfarin/coumadin therapy, subjects were maintained on warfarin/coumadin for at least 3 weeks prior to treatment with an INR ≥2 (to be confirmed maximum 48 hours pre-procedure). Any INR <2 within 3 weeks prior to ablation lead to exclusion of the subject or postponement of the study procedure until the INR is ≥2 for at least 3 weeks prior to treatment (the subject will need to be reconsented if treatment is scheduled for >90 days after original consent date). The results were available prior to start of procedure. NOTE: If a subject was receiving warfarin/coumadin therapy and the anticoagulation therapy is changed to heparin or an equivalent agent due to documented elevated INR, this was considered continuous anticoagulation therapy and the subject proceeded with the study procedure even if the INR fell below 2 while the subject is still receiving alternate therapy.
Physical Exam (including standardized neurological examination): Exam was performed by a physician or equivalent (NP or PA) prior to the ablation procedure. The following items were evaluated as part of the standardized neurological examination: mental status (e.g., orientation, attention, vision, hearing, speech, level of consciousness), cranial nerve testing, assessment of gait and coordination, ability to move all extremities, and evaluation of sensory and motor strength of all 4 extremities. (Note: Subjects in the NAE (neurological assessment evaluation) subset were required to undergo a neurological examination amongst other neurological assessments. Administration of the NAE subset specific neurological exam satisfied the requirements of a standardized neurological examination, and thus, for subjects in the NAE subset, additional standardized neurological examination is not required.)
Vital Signs: height, weight, pulse rate, blood pressure
CV Hospitalization and Cardioversion History: History of all cardiovascular-related hospitalizations, including ER, outpatient, inpatient, and any cardioversions (pharmacological or DC) in the 12 months prior to enrollment.
Electrocardiogram: Data from 12-lead ECG recordings was collected, if available.
Imaging to determine LA size and LVEF: Imaging was required to determine the atrial size and LVEF prior to the study catheter insertion. Transthoracic Echocardiogram (TTE), CT, MRI, ICE, or TEE were allowable imaging modalities to determine LA size and LVEF. If the subject had undergone an imaging procedure within the last 6-months prior to enrollment where the atrial size (parasternal long axis view) and LVEF were assessed and documented, the pre-procedure imaging assessment was not required.
Imaging for detection of LA thrombus (performed within 48 hours of the study procedure or day of ablation procedure prior to study catheter insertion). The following are allowable imaging modalities:
   TEE
   CT/MRI
   Intracardiac Echocardiography (ICE)
Pregnancy Test (within 24 hours prior to the procedure): pre-menopausal women only.
Baseline Quality of Life (QOL) Assessment: AFEQT and CCS-SAF.
Adverse Events were collected from the time the subject signed the informed consent.
Neurological Assessment (NAE Subjects only): The following assessments were required to be performed within 72 hours prior to the ablation procedure for subjects included in the NAE subset, including:
   Cerebral MRI
   Neurological Exam (Note: administration of this NAE neurological exam fulfilled the requirement of the standardized neurological exam and, thus, no additional standardized neurological exam was required.)
   National Institutes of Health Stroke Scale (NIHSS)
   Modified Rankin Scale (mRS)
   Mini-Mental State Examination (MMSE)

NIHSS and mRS assessments was performed by a site staff certified in the administration of the assessments. A certified neurologist, blinded to the cerebral MRI data, performed neurologic exams. Cerebral MRIs were analyzed by a central core lab to determine the frequency, size, and anatomical location of cerebral micro-emboli, if any.

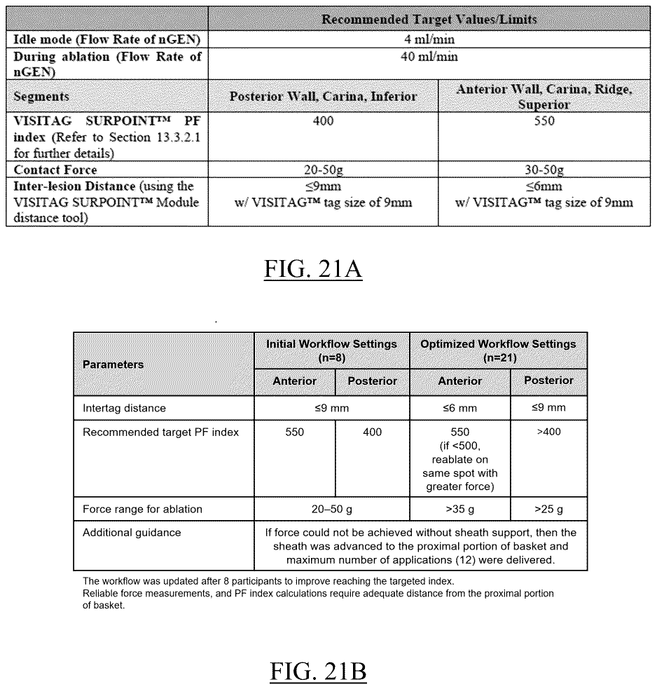

FIGS. 21A and 21B are tables illustrating recommended parameters of completing a procedure with a catheter configured for mapping and ablating tissue, in accordance with an example of the disclosed technology. FIG. 21A shows an initial recommended parameters while FIG. 21B shows both initial parameters (workflow settings) and optimized parameters identified after an initial phase of the study.

As shown in FIG. 21A, irrigation fluid can be delivered to the medical probe 22 at a rate of approximately 4 ml/min when the medical probe 22 is in an idle mode (i.e., not delivering ablative energy) and at a rate of 40 ml/min during ablation.

Depending on the tissue that is to be ablated within the patient's heart, the study recommended delivering ablative energy according to various settings. For example, if tissue at the posterior wall, carina, or inferior were to be ablated, the method included applying ablative energy with an ablation index (PF index) of approximately 400, a contact force of approximately 20-50 grams, and an inter-lesion distance of approximately less than or equal to 9 millimeters with a tag size of approximately 9 millimeters. Alternatively, if tissue at the anterior wall, carina, ridge, or superior were to be ablated, the method included applying ablative energy with an ablation index (PF index) of approximately 550, a contact force of approximately 30-50 grams, and an inter-lesion distance of approximately less than or equal to 6 millimeters with a tag size of approximately 9 millimeters. That is, because different locations of the heart include varying tissue characteristics, applying ablative energy according to predefined pulsed-field ablation patterns (e.g., number of pulses, number of trains of pulses, voltage, etc.) and predefined force as detected by the force sensor can help to ensure proper ablation at the predetermined location.

FIG. 21B illustrates some optimized parameters (workflow settings) that were identified after an initial phase of the study. The optimized parameters similarly include varying recommended parameters depending on the location of the tissue to be ablated. For example, if tissue at the posterior wall was to be ablated, the method included applying ablative energy with an ablation index (PF index) of approximately greater than 400, a contact force of approximately greater than 25 grams, and an inter-lesion distance of approximately less than or equal to 9 millimeters. Alternatively, if tissue at the anterior wall was to be ablated, the method included applying ablative energy with an ablation index (PF index) of approximately 550 (if the PF index was less than 500, it was recommended to reablate on the same spot with a greater force), a contact force of approximately greater than 35 grams, and an inter-lesion distance of approximately less than or equal to 6 millimeters.

In each of the above-described ablation scenarios, if the predetermined force could not be achieved without sheath support, the sheath was advanced to the proximal portion of the basket and the maximum number of ablations of ablative energy (12) were delivered.

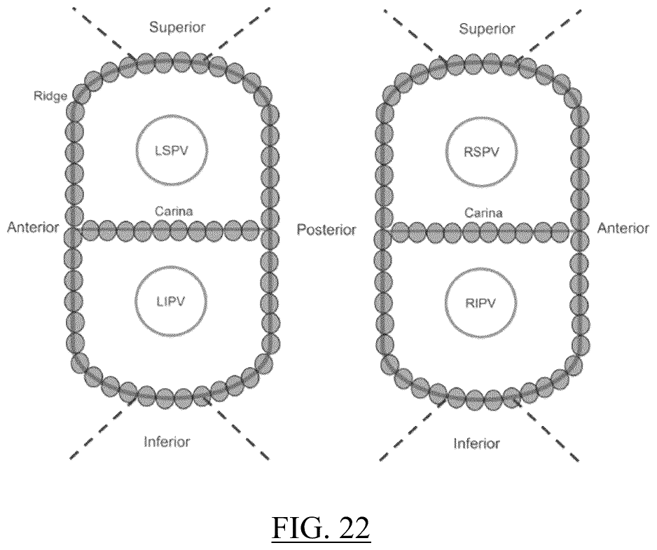

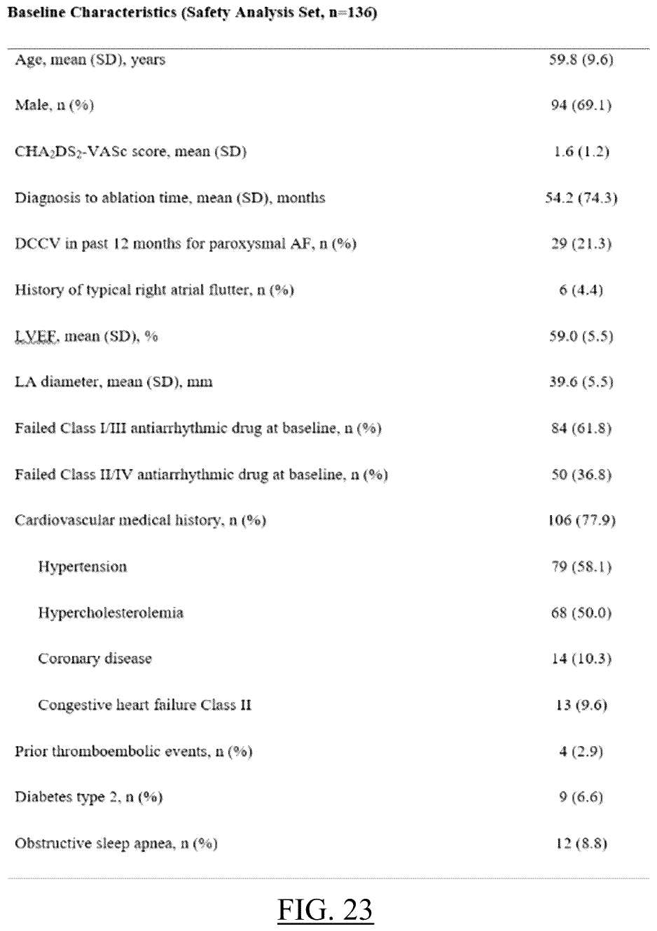

FIG. 22 is a schematic diagram of an example of the various segments within a heart as defined by a VISITAG SURPOINT™ PF Index pulmonary vein segments, in accordance with the various locations within a heart to be ablated as described with respect to FIGS. 21A and 21B.

The paroxysmal atrial fibrillation ablation procedure was completed according to the following sequence: anatomical mapping of the left atrium (LA); pulmonary vein isolation (PVI) using the study catheter; confirmation of PVI in all targeted pulmonary veins (PVs) by documented entrance block with adenosine/isoproterenol challenge; if necessary, treatment of acute reconnections with additional applications of PF energy with the study catheter; if additional applications are applied, and reconfirming PVI by documented entrance block of all PVs. The ablation procedure was considered complete when entrance block is confirmed and documented.

The ablation procedure was completed for each participant in the study according to the following:
REQUIRED: Confirmation of activated clotting time (ACT) ≥350 seconds PRIOR to ablation with the OMNYPULSE™ Catheter.
   ACT MUST be targeted to be maintained ≥350 sec throughout the ablation.
   ACT MUST be checked every 15-30 minutes throughout the ablation.
   If ACT is below 350, heparin should be administered (except for any safety reasons) to ensure an ACT target of 350 seconds without pausing ablation procedure.
REQUIRED: Preparation and introduction of the 10 F compatible guiding sheath per IFU, if not used earlier for mapping. Before inserting the sheath into the patient, flush the sheath with heparinized normal saline to remove air bubbles.
REQUIRED: Ensure that the OMNYPULSE™ Catheter is recognized by CARTO® 3 prior to insertion into the subject's body. If not used earlier for mapping, introduce the OMNYPULSE™ Catheter per IFU.
NOTE: When using a diagnostic device in the same chamber as ablation device, ensure that the device is not in close proximity to the ablation catheter.
Advance and position the OMNYPULSE™ Catheter at the targeted PV.

The recommended workflow for pulmonary vein isolation in the study according to the following:
Use point-by-point to obtain a contiguous lesion set (i.e., no visible gaps) per recommended parameters in Table 13.3.1 and physician training for PV isolation.
   Utilization of the PF index to deliver lesions around the pulmonary veins is REQUIRED.
   NOTE: In the absence of safety concerns, achievement of the recommended target PF index values (Table 13.3.1) is to be used as the endpoint of PF energy application for PV encircling lesions.
   Evaluate the Inter-lesion distance using the VISITAG™ Module distance tool using the recommended parameters in Table 13.3.1.
   Ensure no visible gaps between VISITAG™ tags.
All subjects will undergo PV ablation with the investigational device until PVI is achieved in all PVs:
   ONLY after investigator deems unable to achieve PVI with the investigational device, a commercial (RF) system can be used to complete the procedure (PVI only).
   REQUIRED: Confirmation of entrance block (PVI) of all PVs
   To verify entrance block, analyze electrograms in coronary sinus and/or atrial paced rhythm to confirm that no PV potentials are present.
   Exit block is optional.
If isolation is not obtained, apply additional ablations of PF where needed.
   It is recommended to check all rotations of the CARTO® map to ensure full circumferential co-axial lesions were achieved on all map surfaces.
Recheck all PVs to confirm PVI.
   Post Pulmonary Vein Isolation:
REQUIRED: Administer adenosine/isoproterenol for each PV to rule out dormant conduction.
REQUIRED: Verification of isolation by demonstration of final entrance block for each PV after administration of adenosine/isoproterenol.
   To verify entrance block, analyze electrograms in coronary sinus and/or atrial paced rhythm to confirm that no PV potentials are present.
   REQUIRED: Demonstration of entrance block MUST be confirmed and documented by the OCTARAY® (preferred), LASSO® or PENTARAY® Catheter.
Pilot Phase subjects ONLY: additional demonstration of entrance block MUST be confirmed and documented by the OMNYPULSE™ catheter.
If entrance block is not achieved, review remaining signals and treat acute reconnections (or visual gaps) with additional ablations of PF.
If ablations are needed after administration of adenosine/isoproterenol, reconfirm isolation by documented final entrance block of all PVs.
ONLY after the investigator deems ablation with the OMNYPULSE™ catheter is unable to achieve PVI, a commercial RF system can be used to complete the procedure.
   Note: to prevent any risk to the subject, the BWI PFA system should be completely disconnected prior to use of an RF system.
The ablation procedure is considered complete when entrance block is confirmed and documented for all PVs.

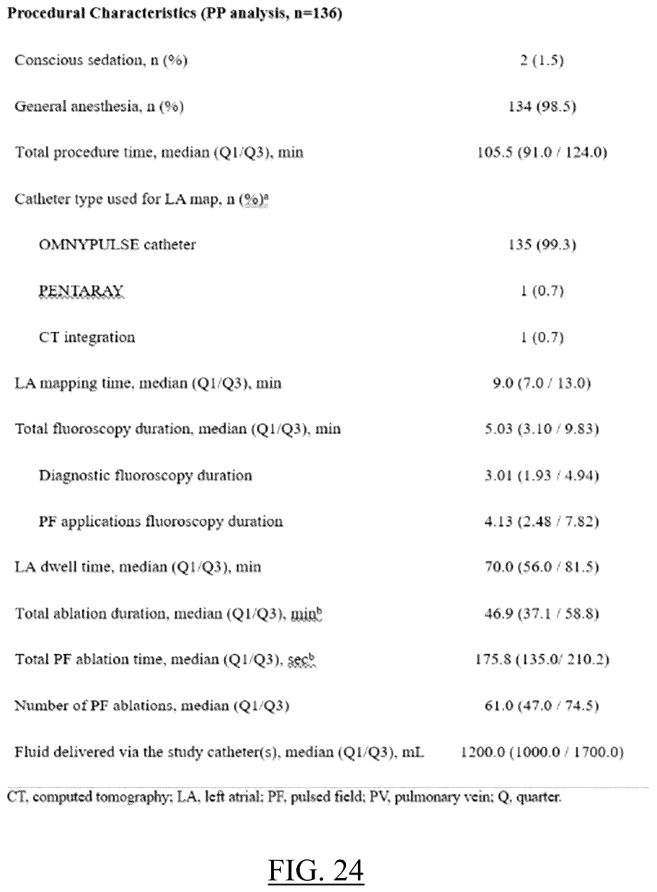

FIG. 23 illustrates baseline characteristics of participants in the study for the safety analysis set of patients, n=136, while FIG. 24 illustrates the procedural characteristics of the safety analysis set.

### Results

Of 188 patients enrolled in the study across 13 centers, with procedures performed by 21 operators, the 136 main phase patients (59.8±9.6 years old, 69.1% male) were included in the per protocol analysis. Acute procedural success was 100% (136/136). Following adenosine/isoproterenol challenge during the index procedure, 16/545 (2.9%) of targeted veins in 10/136 (7.4%) patients showed acute reconnection, resulting in a first-pass isolation rate of 97.1% per vein and 92.6% per patient. The primary effectiveness endpoint was met because the lower bound of the exact two-sided 95% confidence interval was 97.3%, which exceeded the prespecified performance goal (90%). Median (Q1/Q3) procedure, LA dwell, pulmonary vein isolation (PVI) ablation, and total fluoroscopy times were 105.5 (91.0 of 124.0), 70.0 (56.0 of 81.5), 48.5 (37.5 of 59.0), and 5.0 (3.1 of 9.8) minutes respectively. The primary adverse event rate was 3.0% (4 of 135, 3 major vascular access complications, 1 pericarditis) with all primary adverse events being resolved.

To further delineate safety and assess lesion durability, subsets of patients underwent additional prospective testing 2-3 months postablation for neurological evaluation for cerebral lesion including silent cerebral lesion, esophageal endoscopy, cardiac computed tomography (CT) or magnetic resonance angiogram (MRA) imaging for PV stenosis, and mandatory remapping for PVI durability assessment. Patients in the PVI durability assessment subset underwent repeat electroanatomic creation of paced activation and bipolar voltage maps at 75 days (±15 days) after the index ablation to verify isolation durability of the treated PV, using the same mapping catheter as that used during the index ablation procedure. In analysis of the subset of patients that underwent systematic brain imaging, esophageal endoscopy, cardiac CT/MRA, and mandatory 3M remapping for PVI durability, 3.3% (1 of 30) presented with an asymptomatic silent cerebral event at discharge which resolved at one month without neurological change. An investigation revealed that 5 intraprocedural catheter exchanges may have resulted in the introduction of bubbles during the case. No esophageal injury was observed in the endoscopy subset (n=30). Of the 101 veins in 24 patients in this subset, 39 (38.6%) PVs showed no narrowing, 59 (58.4%) PVs had mild narrowing of >0 to 25%, and 3 (3.0%) PVs had mild narrowing of >25 to 50% at the 3-month CT/MRA. There were no instances of moderate (>50-70%) or severe (>70%) PV narrowing. At the patient level, 2 (8.3%) patients had no PV narrowing, 20 (83.3%) patients had mild narrowing of >0 to 25%, and 2 (8.3%) patients had mild narrowing of >25 to 50% at the 3-month follow-up. During remapping procedures (at 81.7±9.1 days post-PFA), pulmonary vein isolation was durable in 84.5% (98 of 116) of veins and 62.1% (18 of 29) of patients. After workflow optimization, PVI durability increased to 89.3% (75 of 84) and 71.4% (15 of 21) per vein and patients, respectively. Esophageal endoscopy was performed at 1 to 3 days post ablation in 29 patients. No esophageal thermal lesions (0/29, 0%) were observed.

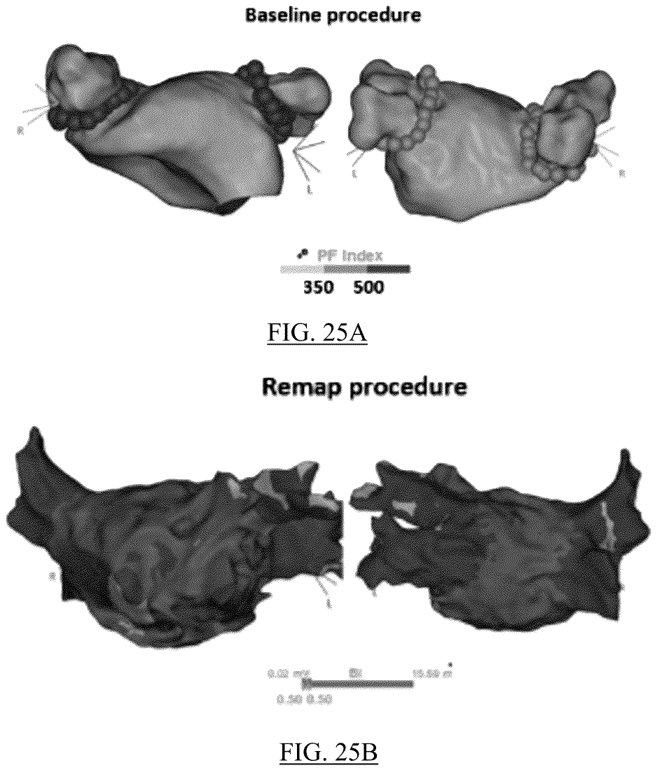

A prespecified subset of 29 patients underwent a mandatory 3D electroanatomic remapping at a mean (SD) 81.7 (9.1) days postindex procedure. Activation and voltage mapping were combined to assess vein isolation. At the time of the remapping study, 26 out of 29 patients were free of atrial tachyarrhythmia. Durable PVI was verified in 98/116 (84.5%) veins and 18/29 (62.1%) patients. In optimized workflow cases (n=21), durable PVI was verified in 75/84 (89.3%) veins and 15/21 (71.4%) patients. In arrhythmia-free cases (n=26), durable PVI was verified in 93/104 (89.4%) veins and 18/26 (69.2%) patients. A representative example of durable isolation in all 4 veins is shown in FIG. 25B with the baseline procedure being shown in FIG. 25A.

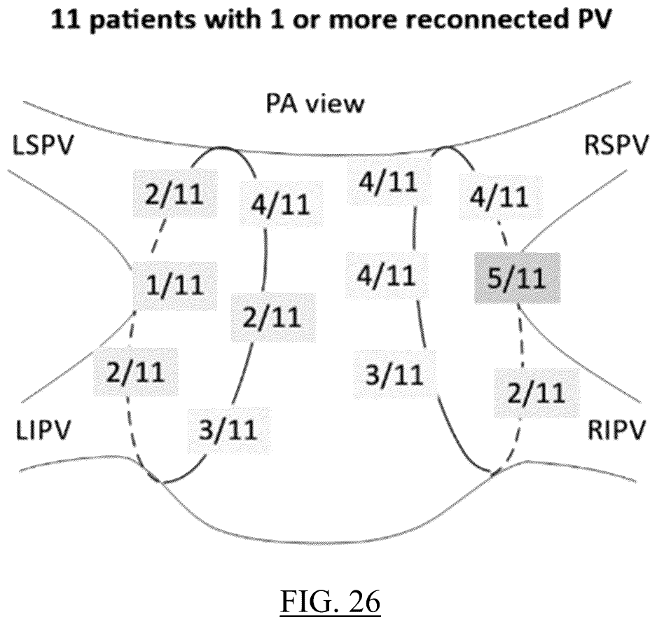

Overall, reconnection was observed in 11 patients and 18 veins. Reconnection of 1 vein-only, 2 veins, or ≥3 veins, was observed in 7, 2, and 2 patients, respectively. The spatial distribution of the locations of reconnections is shown in FIG. 26.

Overall, 36 regions revealed a conduction gap (3.3 gaps per patient). There was a trend towards more reconnection at right-sided veins (22 vs 14 gaps), with a trend towards more reconnection at the anterior and posterior carina.

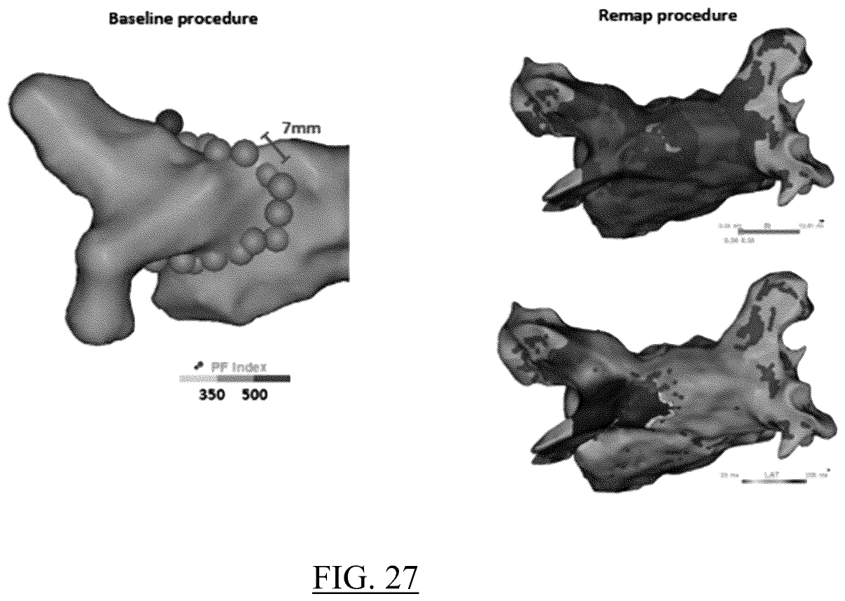

In 33 of 36 gaps, the region of reconnection corresponded to a region containing an intertag distance of 6 mm or more during the index procedure (9.3±1.7 mm). A representative example is given in FIG. 27. In this patient, the LSPV was reconnected due to a gap at the posterosuperior region of the left circle. Revisiting the ablation tags at the index map revealed an intertag distance of 7 mm at that same region.

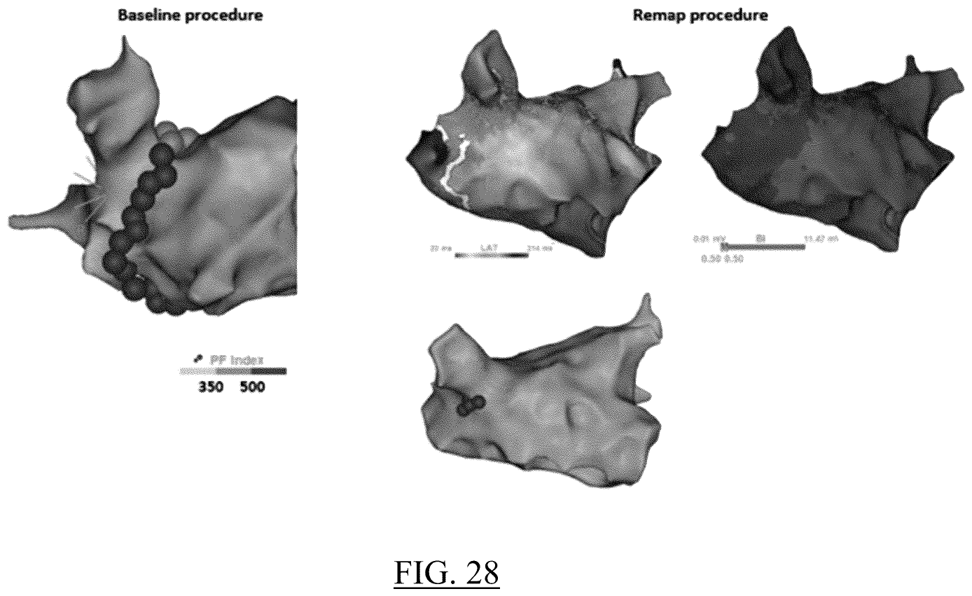

In 3 of 36 gaps, reconnection occurred in a region that was ablated strictly according to the protocol. A representative example is given in FIG. 28. In this patient, the RSPV and RIPV were reconnected due to a gap at the anterior carina of the right circle. Revisiting the ablation tags at the index map revealed therapeutic PF index and intertag distance at that same region.

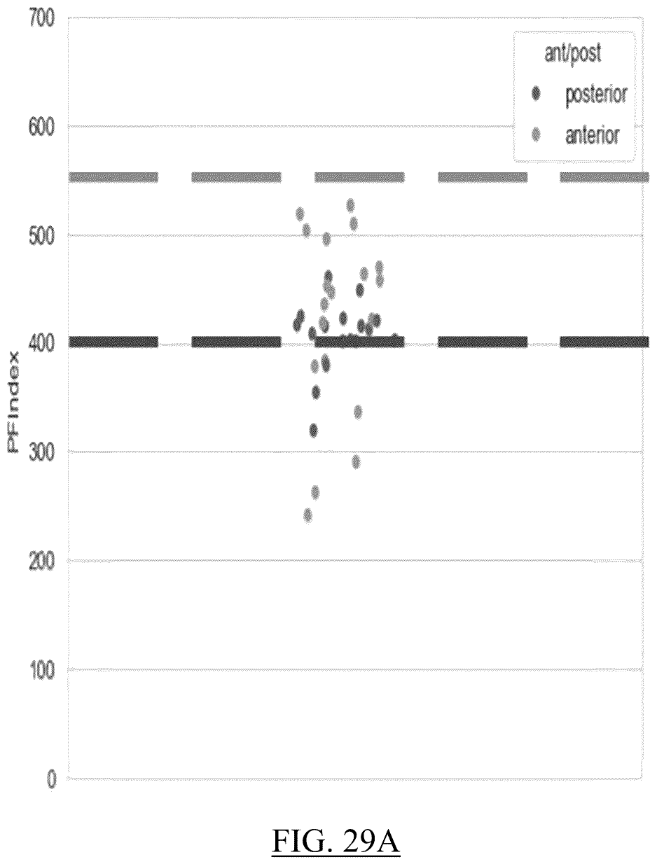

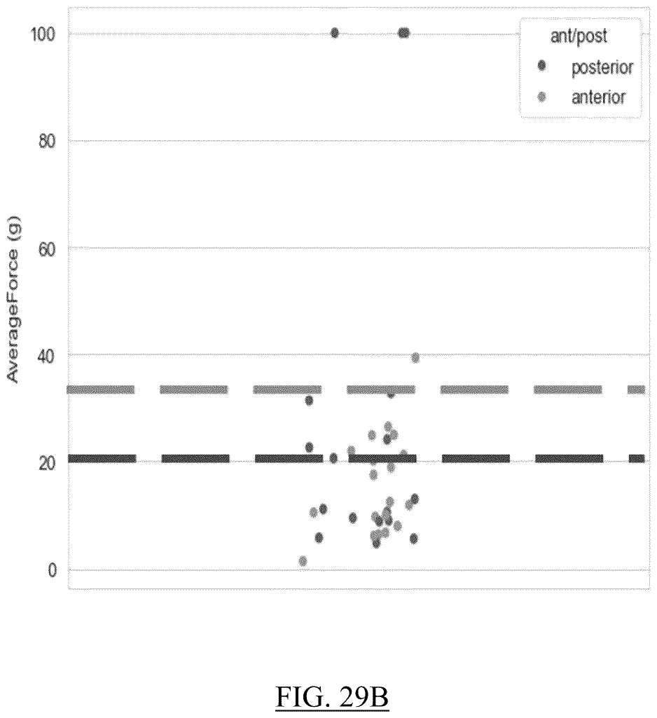

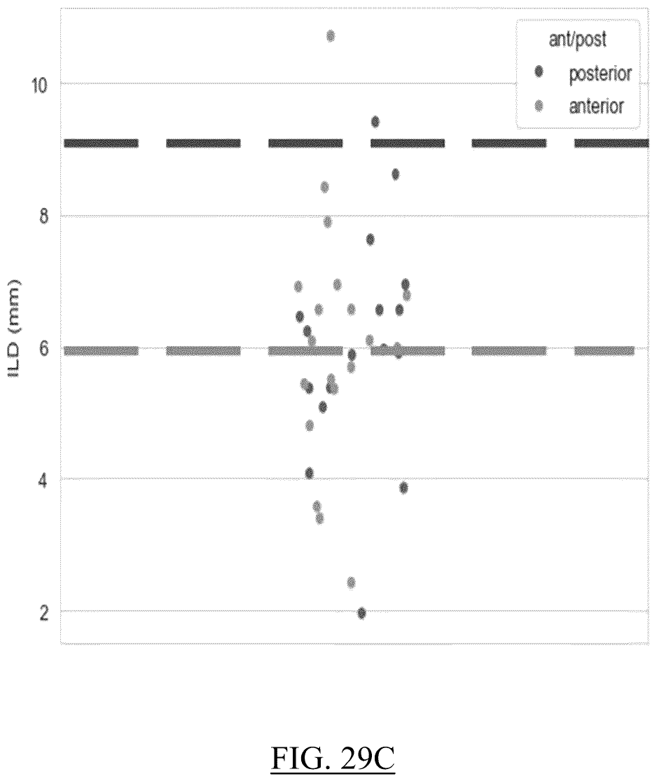

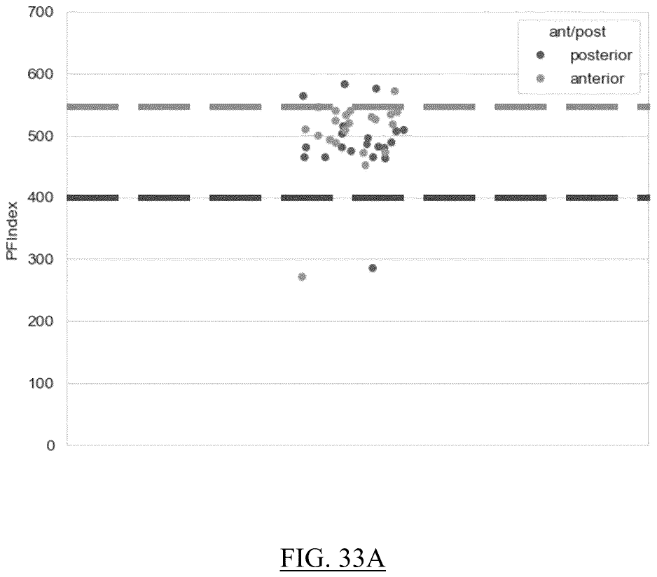

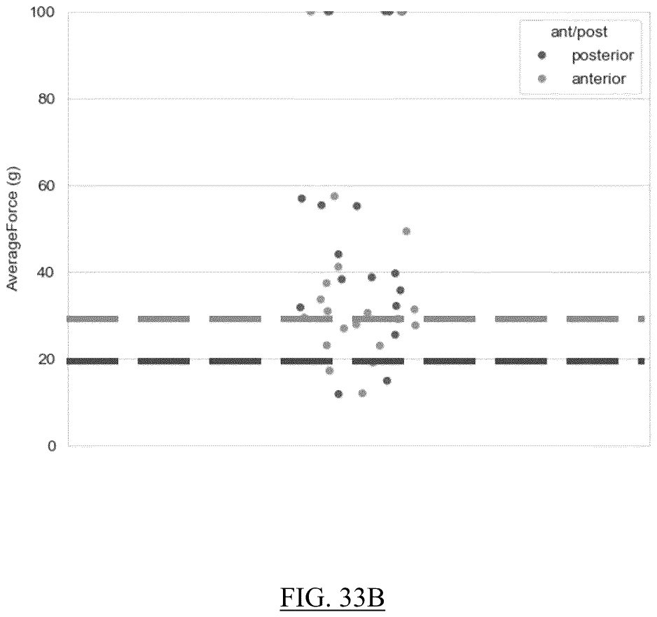

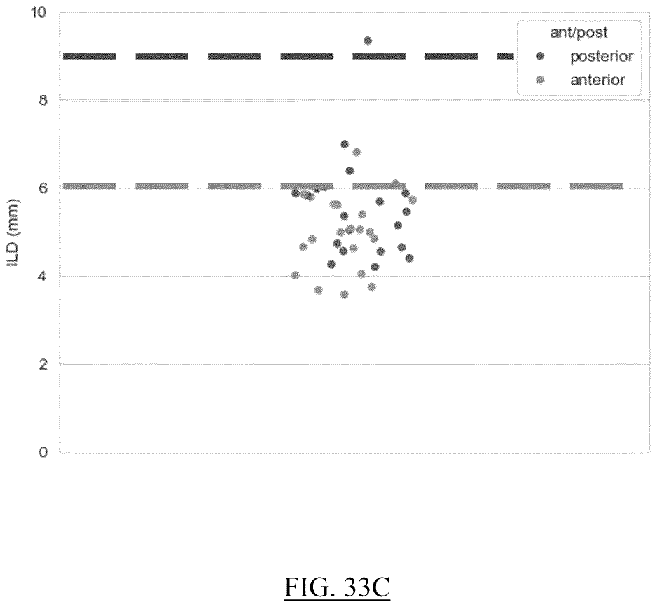

FIGS. 29A-32B illustrate graphs and electroanatomical models of a patient's heart included in the study according to a first example while FIGS. 33A-36C illustrate graphs and electroanatomical models of a patient's heart included in the study according to a second example. These examples are offered to illustrate the effect of applying ablative energy according to various ranges of PF Indexes, force, and inter lesion distances. It will become apparent throughout these illustrative examples that the PF Indexes, forces, and inter lesion distances described previously herein are specifically selected to ensure proper ablation and isolation of aberrant electrical signals propagating through tissue in a heart. These examples should not be construed as limiting but are offered solely as illustrative of the technology disclosed herein.

FIG. 29A illustrates various PF Indexes recorded during an ablation procedure of a first patient. Orange dots represent ablation of an anterior portion while blue dots represent ablation of a posterior portion of the patient's heart. The orange dashed line represents the recommended PF Index of 550 for an anterior procedure while the blue dashed line represents the recommended PF Index of 400 for a posterior procedure. As shown, the PF Indexes recorded during this particular procedure were below the recommended PF Indexes for the respective procedure.

FIG. 29B illustrates average force values recorded during the ablation procedure of the first patient. As before, orange dots represent ablation of an anterior portion while blue dots represent ablation of a posterior portion of the patient's heart. The orange dashed line represents the recommended force of 35 grams or greater for an anterior procedure while the blue dashed line represents a force of 20 grams for a posterior procedure. As shown, the forces recorded during this particular procedure were below the recommended forces for the respective procedure.

FIG. 29C illustrates the inter-lesion distances recorded during the ablation procedure of the first patient. As before, orange dots represent ablation of an anterior portion while blue dots represent ablation of a posterior portion of the patient's heart. The orange dashed line represents the recommended inter-lesion distance of 6 millimeters for an anterior procedure while the blue dashed line represents the recommended inter-lesion distance of 9 millimeters for a posterior procedure. As shown, the inter-lesions distances recorded during this particular procedure were rather inconsistent with many of the many of the inter-lesion distances being greater than the recommended inter-lesion distance for the respective procedure.

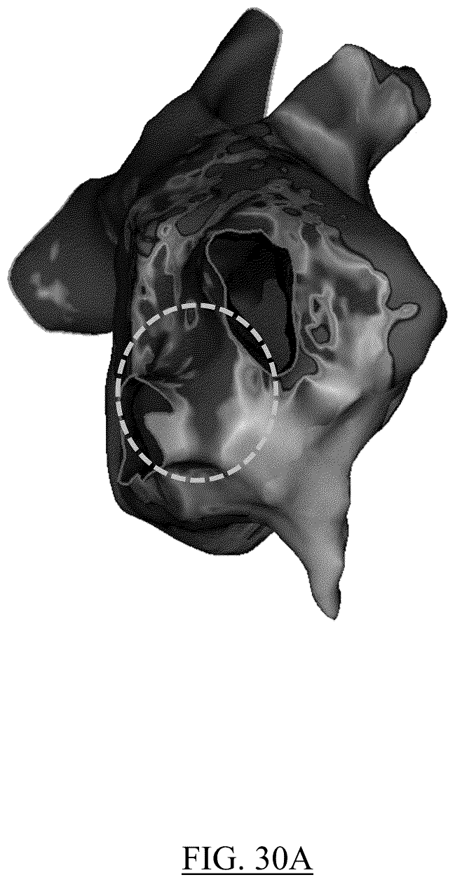

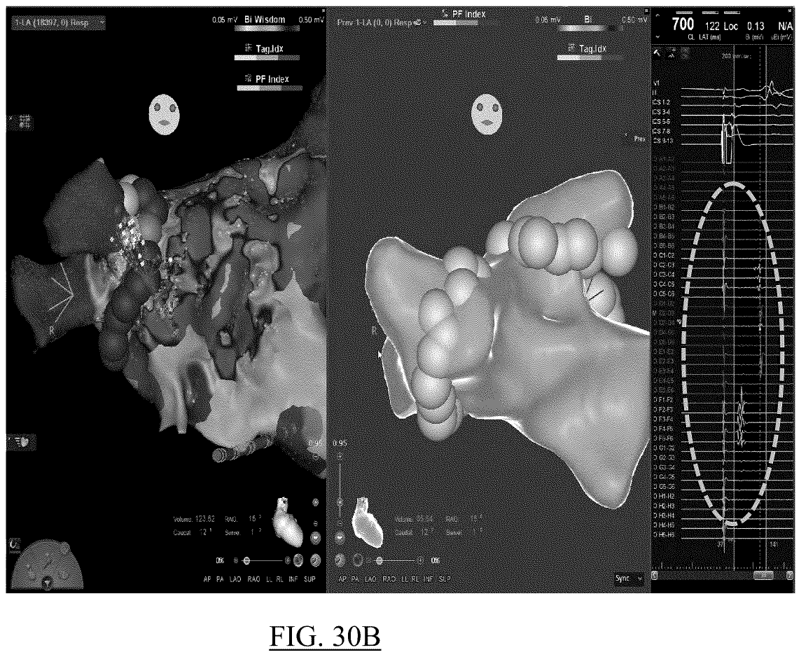

FIG. 30A illustrates an electroanatomical map recorded of the first patient's heart after the procedure performed with the recorded parameters shown and described in FIGS. 29A-29C. As shown by the area circled by the orange dashed line, a reconnection was identified in the pulmonary vein after the procedure. In this particular procedure, reconnection was identified in the right superior pulmonary vein as well as the right inferior pulmonary vein, thereby requiring a subsequent ablation to isolate the pulmonary veins.

FIG. 30B illustrates an electro anatomical map and electrogram recording shown the reconnection identified in FIG. 30A. The orange dashed line in FIG. 30B highlights the recorded electrograms.

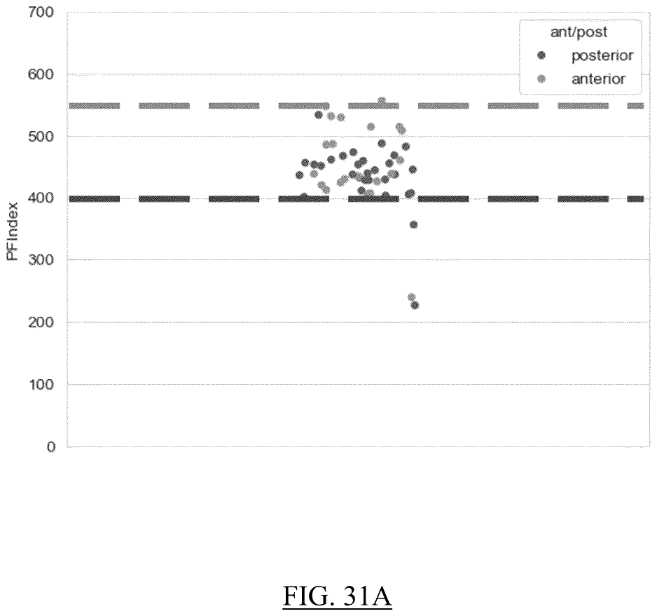

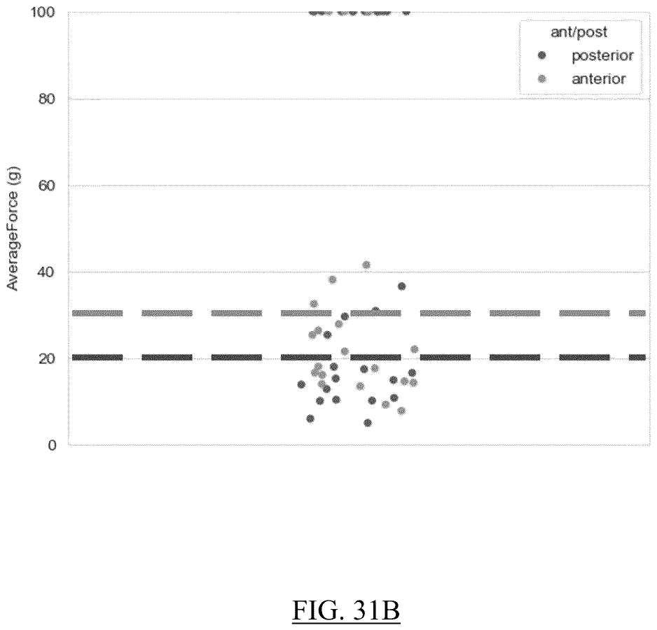

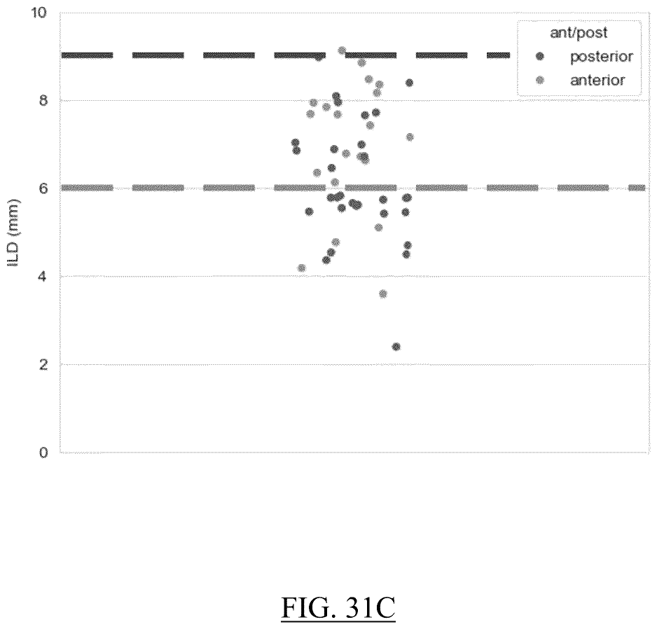

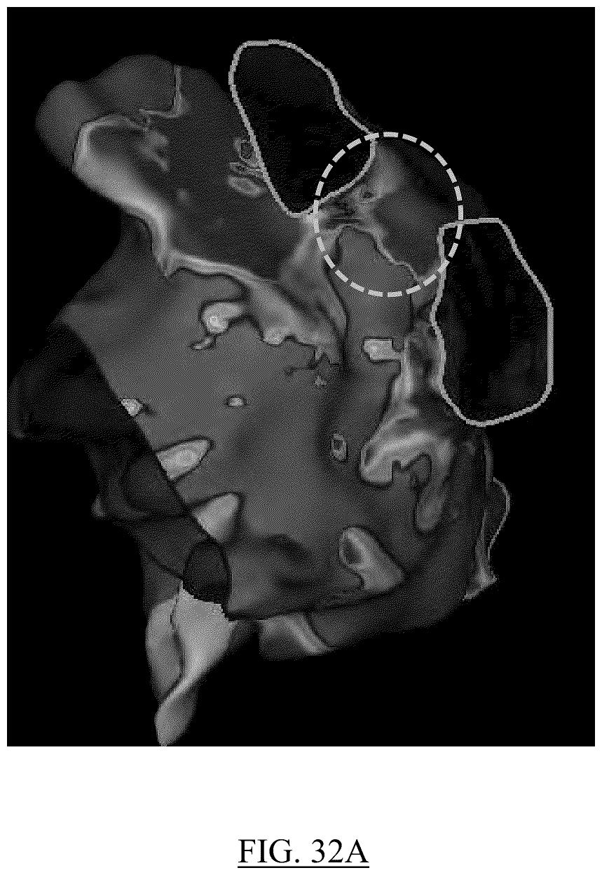

FIG. 31A illustrates various PF Indexes recorded during an ablation procedure of a second patient. Orange dots represent ablation of an anterior portion while blue dots represent ablation of a posterior portion of the patient's heart. The orange dashed line represents the recommended PF Index of 550 for an anterior procedure while the blue dashed line represents the recommended PF Index of 400 for a posterior procedure. As shown, many of the PF Indexes recorded during this second procedure were below the recommended PF Indexes at least for the anterior procedure.

FIG. 31B illustrates average force values recorded during the ablation procedure of the second patient. As before, orange dots represent ablation of an anterior portion while blue dots represent ablation of a posterior portion of the patient's heart. The orange dashed line represents the recommended force of 35 grams or greater for an anterior procedure while the blue dashed line represents a force of 20 grams for a posterior procedure. As shown, many of the forces recorded during this second procedure were below the recommended forces for the respective procedure.

FIG. 31C illustrates the inter-lesion distances recorded during the ablation procedure of the second patient. As before, orange dots represent ablation of an anterior portion while blue dots represent ablation of a posterior portion of the patient's heart. The orange dashed line represents the recommended inter-lesion distance of 6 millimeters for an anterior procedure while the blue dashed line represents the recommended inter-lesion distance of 9 millimeters for a posterior procedure. As shown, the inter-lesions distances recorded during this particular procedure were rather inconsistent with many of the inter-lesion distances being greater than the recommended inter-lesion distance at least for the anterior portion of the procedure.

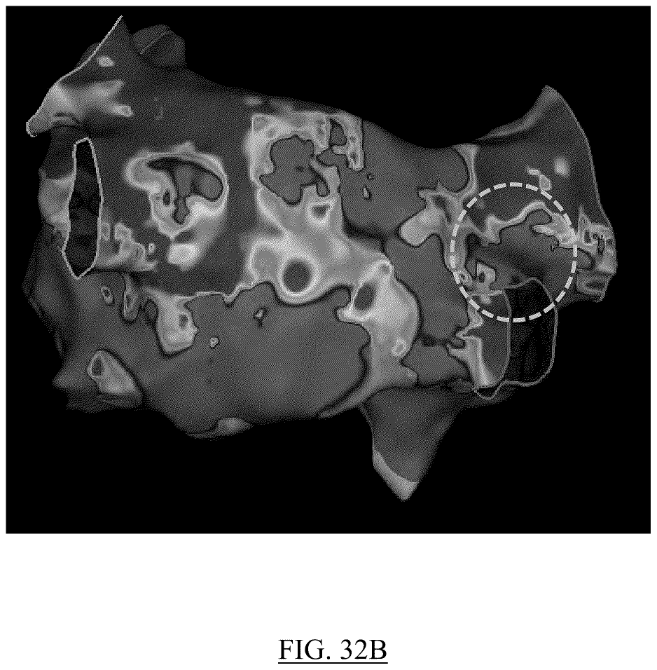

FIG. 32A illustrates an electroanatomical map recorded of the second patient's heart after the procedure performed with the recorded parameters shown and described in FIGS. 31A-31C. As shown by the area circled by the orange dashed line in FIGS. 32A and 32B, reconnections were identified in the pulmonary veins after the procedure. In this particular procedure, reconnection was identified in all four pulmonary veins, thereby requiring a subsequent ablation to isolate the pulmonary veins.

FIG. 33A illustrates various PF Indexes recorded during an ablation procedure of a third patient. Orange dots represent ablation of an anterior portion while blue dots represent ablation of a posterior portion of the patient's heart. The orange dashed line represents the recommended PF Index of 550 for an anterior procedure while the blue dashed line represents the recommended PF Index of 400 for a posterior procedure. As shown, many of the PF Indexes recorded during this particular procedure were below, but at least closer to the recommended 550 PF Index for the anterior portion of the procedure. Furthermore, many of the recorded PF Indexes for the posterior portion of the procedure were greater than the recommended PF Index of 400.

FIG. 33B illustrates average force values recorded during the ablation procedure of the third patient. As before, orange dots represent ablation of an anterior portion while blue dots represent ablation of a posterior portion of the patient's heart. The orange dashed line represents the recommended force of 35 grams or greater for an anterior procedure while the blue dashed line represents a force of 20 grams for a posterior procedure. As shown, many of the forces recorded during this particular procedure were greater than the recommended forces for the respective procedure.

FIG. 33C illustrates the inter-lesion distances recorded during the ablation procedure of the third patient. As before, orange dots represent ablation of an anterior portion while blue dots represent ablation of a posterior portion of the patient's heart. The orange dashed line represents the recommended inter-lesion distance of 6 millimeters for an anterior procedure while the blue dashed line represents the recommended inter-lesion distance of 9 millimeters for a posterior procedure. As shown, most of the inter-lesions distances recorded during this particular procedure were less than the recommended inter-lesion distance for the respective procedure.

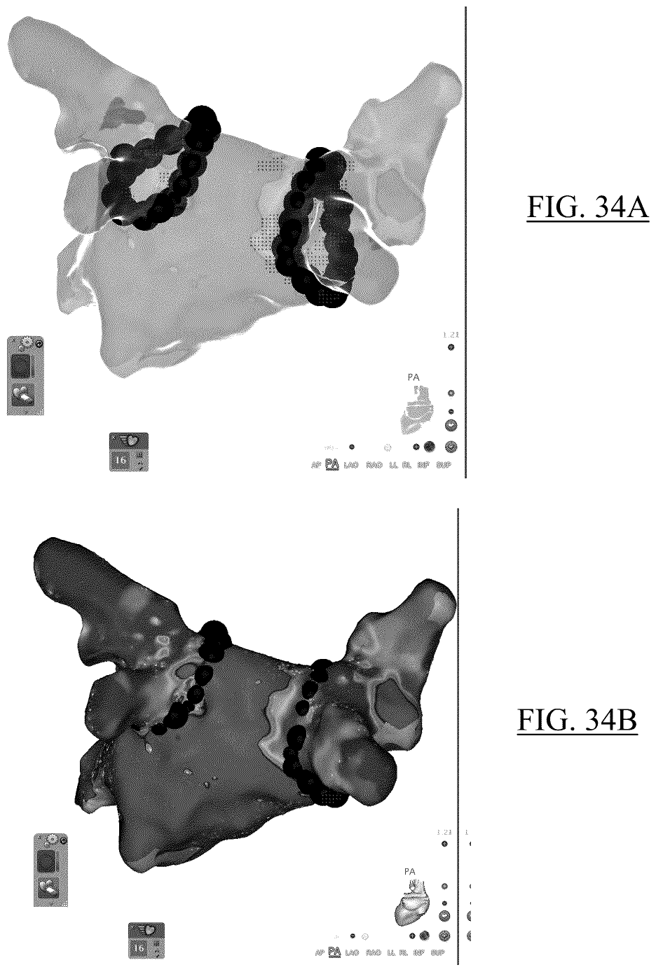

FIGS. 34A and 34B illustrates an electroanatomical map recorded of the third patient's heart after the procedure performed with the recorded parameters shown and described in FIGS. 33A-33C. As shown, the total isolation was achieved through the ablation procedure completed for this third patient.

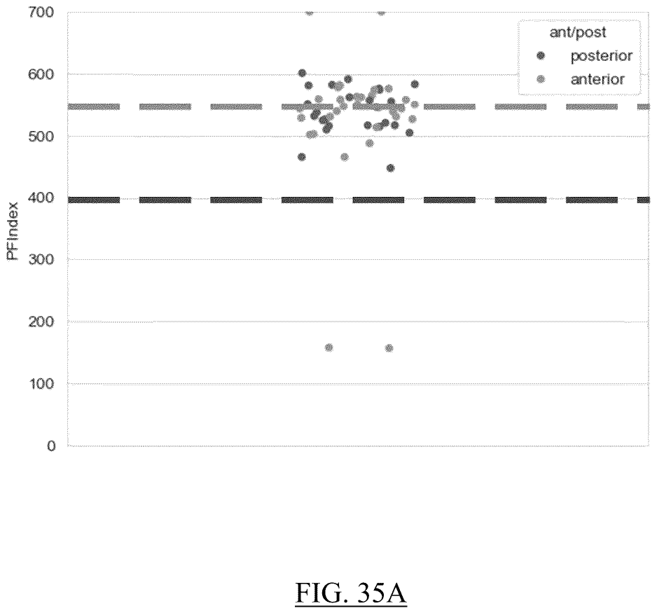

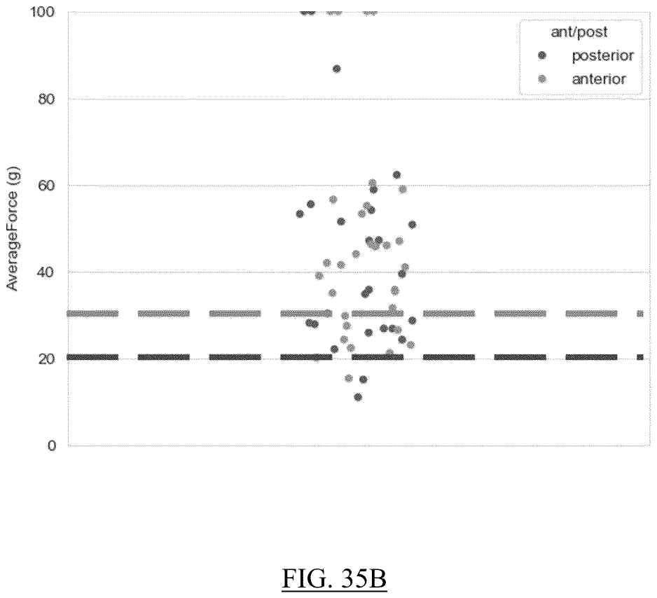

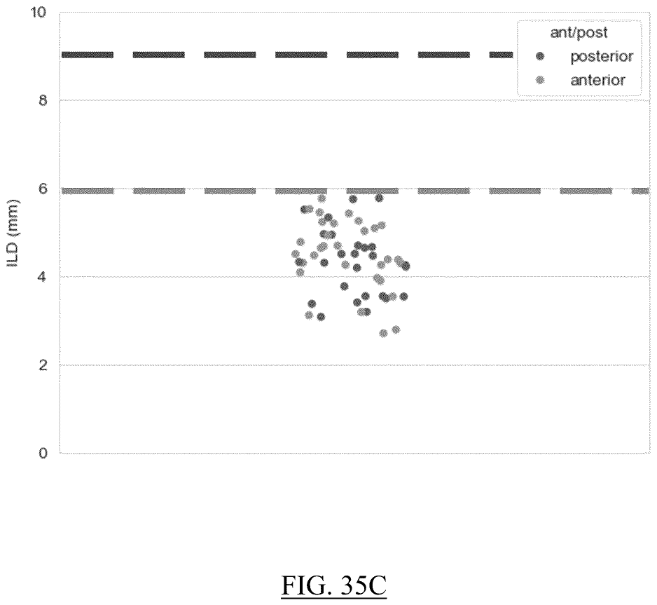

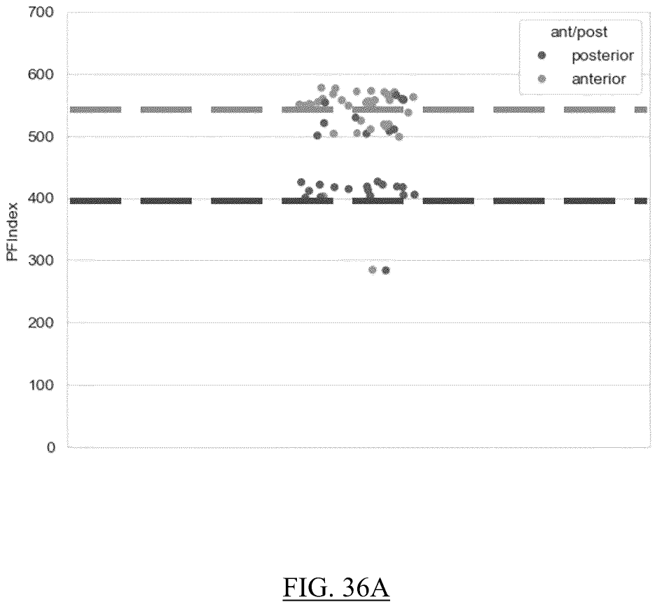

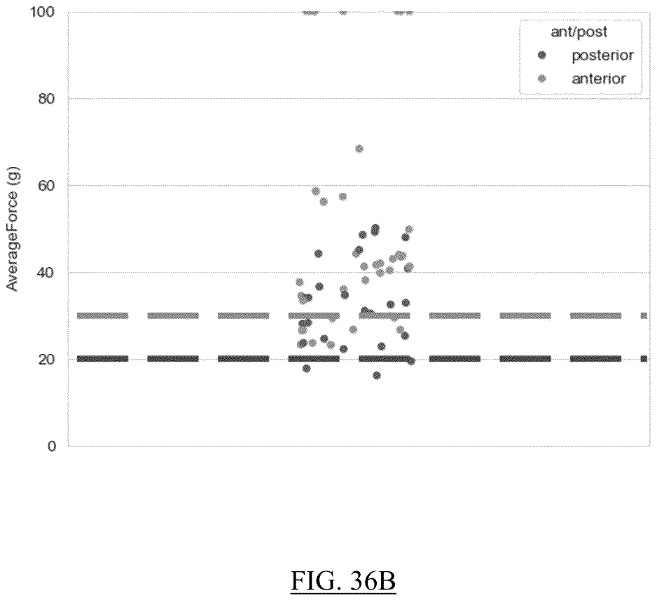

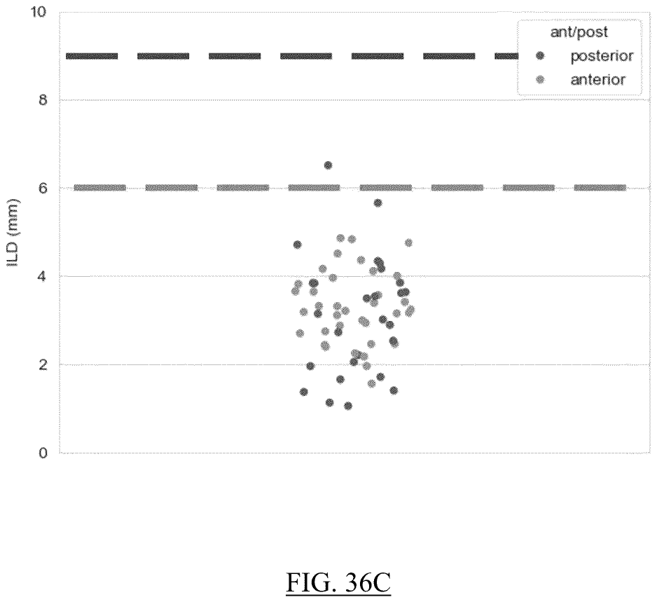

FIGS. 35A-36C illustrate additional recorded PF Index, average force, and inter-lesion distance for a fourth patient and a fifth patient, respectively. Although not shown, complete isolation of the pulmonary veins was achieved during the ablation procedures for both the fourth patient and the fifth patient.

FIG. 35A illustrates various PF Indexes recorded during an ablation procedure of a fourth patient. Orange dots represent ablation of an anterior portion while blue dots represent ablation of a posterior portion of the patient's heart. The orange dashed line represents the recommended PF Index of 550 for an anterior procedure while the blue dashed line represents the recommended PF Index of 400 for a posterior procedure. As shown, many of the PF Indexes recorded during this particular procedure were close to or greater than the recommended 550 PF Index for the anterior portion of the procedure and the recommended PF Index of 400 for the posterior portion of the procedure.

FIG. 35B illustrates average force values recorded during the ablation procedure of the fourth patient. As before, orange dots represent ablation of an anterior portion while blue dots represent ablation of a posterior portion of the patient's heart. The orange dashed line represents the recommended force of 35 grams or greater for an anterior procedure while the blue dashed line represents a force of 20 grams for a posterior procedure. As shown, many of the forces recorded during this particular procedure were greater than the recommended forces for the respective procedure.

FIG. 35C illustrates the inter-lesion distances recorded during the ablation procedure of the fourth patient. As before, orange dots represent ablation of an anterior portion while blue dots represent ablation of a posterior portion of the patient's heart. The orange dashed line represents the recommended inter-lesion distance of 6 millimeters for an anterior procedure while the blue dashed line represents the recommended inter-lesion distance of 9 millimeters for a posterior procedure. As shown, all of the inter-lesions distances recorded during this particular procedure were less than the recommended inter-lesion distance for the respective procedure (all less than 6 mm). As previously stated, complete isolation of the pulmonary veins was achieved through the ablation procedure performed on the fourth patient.

FIG. 36A illustrates various PF Indexes recorded during an ablation procedure of a fifth patient. Orange dots represent ablation of an anterior portion while blue dots represent ablation of a posterior portion of the patient's heart. The orange dashed line represents the recommended PF Index of 550 for an anterior procedure while the blue dashed line represents the recommended PF Index of 400 for a posterior procedure. As shown, many of the PF Indexes recorded during this particular procedure were close to or greater than the recommended 550 PF Index for the anterior portion of the procedure and the recommended PF Index of 400 for the posterior portion of the procedure.

FIG. 36B illustrates average force values recorded during the ablation procedure of the fifth patient. As before, orange dots represent ablation of an anterior portion while blue dots represent ablation of a posterior portion of the patient's heart. The orange dashed line represents the recommended force of 35 grams or greater for an anterior procedure while the blue dashed line represents a force of 20 grams for a posterior procedure. As shown, many of the forces recorded during this particular procedure were greater than the recommended forces for the respective procedure.

FIG. 36C illustrates the inter-lesion distances recorded during the ablation procedure of the fifth patient. As before, orange dots represent ablation of an anterior portion while blue dots represent ablation of a posterior portion of the patient's heart. The orange dashed line represents the recommended inter-lesion distance of 6 millimeters for an anterior procedure while the blue dashed line represents the recommended inter-lesion distance of 9 millimeters for a posterior procedure. As shown, almost all of the inter-lesions distances recorded during this particular procedure were less than the recommended inter-lesion distance for the respective procedure (all less than 6 mm). As previously stated, complete isolation of the pulmonary veins was achieved through the ablation procedure performed on the fifth patient.

As will be appreciated, the data shown and described with respect to FIGS. 29A-36C illustrate that the recommended parameters (e.g., PF Index, force, inter-lesion distance) shown and described herein can be used to effectively achieve durable pulmonary vein isolation using the catheter disclosed herein.

The large focal pulsed field ablation catheter with 3D electro-anatomical mapping integration shows 100% acute success with a promising safety profile in the treatment of paroxysmal atrial fibrillation. Prespecified 3M remapping showed high pulmonary vein isolation durability.

Specific advantages of the focal pulsed field ablation catheter with 3D electro-anatomical mapping include its bipolar nature, mapping quality, and high stability, allowing for ablation deeper at the carina. In addition, as a large-tip focal catheter with integrated electroanatomic mapping, the disclosed catheter enables more personalized ablation strategies compared with multielectrode catheters.

### Efficacy Index

The disclosed technology further includes a system and method of determining whether the medical treatment (i.e., an ablation procedure) is likely to be successful and prevent the patient from experiencing symptomatic or asymptomatic recurrence of one or more cardiovascular abnormalities. For example, the disclosed technology can include a method of determining whether a particular treatment is likely to prevent the patient from experiencing atrial fibrillation, atrial tachyarrhythmia, or atrial flutter, or similar cardiac issues. That is, in contrast to how the PFA AI is configured to determine the quality or effectiveness of one particular lesion formed during an ablation procedure, the disclosed technology can further include an efficacy index that is configured to determine how multiple lesions formed during a cardiac ablation would, as a whole, prevent the recurrence of cardiovascular abnormalities. This can be particularly helpful for predicting a long-term success of a particular cardiac ablation so that a physician is able to determine the likelihood that the patient will be free from recurrence of one or more cardiovascular abnormalities.

To illustrate, the disclosed technology includes a method and system that enables a physician to insert a catheter into a patient's heart and determine in real time whether the particular ablation treatment is effectively treating the cardiovascular abnormality. As the physician is completing the cardiac ablation and forming lesions with the medical probe, the system can will determine an efficacy index that comprises a prediction of whether the patient is likely to experience recurrence of the cardiovascular abnormality after treatment. The efficacy index is automatically updated after each lesion is formed during the cardiac ablation such that the physician is able to receive real time feedback indicative of how effective the cardiac ablation is. That is, the processor 46 can automatically present to medical professional 34 on the display 64 an indication (such as a number, percentage value, color (e.g., red, green, yellow), etc.) that indicates whether the cardiac ablation, based on the lesions formed during treatment so far, is likely to be effective for preventing recurrence in the long term.

The efficacy index can be determined by receiving or collecting (as well as storing) data from multiple sensors and the ablation generator to determine the location, size, depth, etc. of each lesion and output an indication of how effective the overall treatment is with multiple lesions. In some examples, the efficacy index can be based on data representative of the contact force, ablation index, and inter-lesion distance of each of the lesions formed during treatment. Because the efficacy index can be updated in real time after formation of each lesion, the physician can be better suited to determine whether the cardiac ablation at any given point during the operation is likely to prevent recurrence of the cardiovascular abnormality.

In some examples, the efficacy index can be calculated according to the following formula and return a value between 0 and 1, a percentage between 0 and 100, and/or another indication. The efficacy index can be represented according to the following formula:

E=(1/(1+exp(−A(PF Index−B))))*(1−(1/(1+exp(−C(ILD−D)))))*(1/(1+exp(−H(CF−I))))

Where:
a. E=efficacy index
b. A=~1
c. PF Index=the ablation index
d. B=~471.5
e. C=~1
f. ILD=the inter-lesion distance (i.e., distance between adjacent lesions)
g. D=~4.7
h. H=~1
i. CF=the contact force, and
j. I=~33.9.

The forgoing values were determined based on data obtained from a set of patients treated with the cardiac ablation described herein. Based on the data and values obtained during treatment as well as follow-up evaluations twelve months after treatment, the efficacy index could be derived to show the long-term success of a given cardiac ablation. Some of the values were determined as curve-fitting values according to the following:
a. B=a midpoint of ablation index values according to the following formula: [((no recurrence PFI mean)−(yes recurrence PFI mean))/2)+(yes recurrence PFI mean)]
b. D=a midpoint of inter-lesion distances according to the following formula: [((no recurrence DistAvg mean)−(yes recurrence DistAvg mean))/2)+(yes recurrence DistAvg mean)]
c. I=a midpoint of contact force values according to the following formula: [((no recurrence CF median)−(yes recurrence CF median))/2)+(yes recurrence CF median)]

As will be appreciated, by considering the values of the contact force, inter-lesion distance, and ablation index for each individual lesion formed during a particular treatment, the efficacy index can return a value providing an indication whether a particular treatment is likely to have been effective for preventing recurrence of the cardiovascular abnormality. In general, as the contact force increases at or above a target contact force, the lesion formed by delivering ablative energy to the tissue via the electrodes is more likely to be sufficient for achieving the desired outcome of the cardiac ablation. Similarly, as the inter-lesion distance decreases (the lesions become closer together), the lesions are more likely to work together to block aberrant electrical signals through tissue and, therefore, the cardiac ablation is more likely to be effective. Further, as the ablation index increases for a lesion, the lesion formed by delivering ablative energy to the tissue via the electrodes is more likely to be sufficient for achieving the desired outcome of the cardiac ablation. Thus, by using the efficacy index to develop an overall picture of the cardiac ablation, there is a higher likelihood that the physician will be able to determine whether the treatment has been effective before terminating the procedure.

The target contact force, target inter-lesion distance, and target ablation index can vary depending on the type and location of the cardiovascular abnormality. For example, for cardiovascular abnormalities located in a posterior portion of a patient's heart, the target contact force can be approximately greater than or equal to 20 grams, the target inter-lesion distance can be approximately less than or equal to 9 millimeters, and the target ablation index can be approximately greater than or equal to 400. In contrast, for cardiovascular abnormalities located in an anterior portion of a patient's heart, the target contact force can be approximately greater than or equal to 30 grams, the target inter-lesion distance can be approximately less than or equal to 6 millimeters, and the target ablation index can be approximately greater than or equal to 500. In some examples, a physician can be able to pre-set the target contact force, target inter-lesion distance, and target ablation index for a particular cardiovascular abnormality to ensure the efficacy index properly reflects whether the cardiac ablation is likely to be effective for a particular patient having a particular cardiovascular abnormality.

The efficacy index can be further improved by factoring how stable a physician was able to hold the end effector when performing ablation. The stability can be determined using data from position sensors disposed on the end effector or near the end effector. Thus, the efficacy index, including the stability values, can be defined by the following formula:

E=(1/(1+exp(−A(PF Index−B))))*(1−(1/(1+exp(−C(ILD−D)))))*(1−(1/(1+exp(−F(Stability−G)))))*(1/(1+exp(−H(CF−I))))

Where:
a. E=efficacy index
b. A=~1
c. PF Index=the ablation index
d. B ~471.5
e. C ~1
f. ILD=the inter-lesion distance (i.e., distance between adjacent lesions)
g. D ~4.7
h. F ~16
i. Stability=the stability of the end effector during ablation
j. G ~2.3
k. H=1
l. CF=the contact force, and
m. I ~33.9.

As will be appreciated, the values for many of the variables in this second efficacy equation are the same as the first equation and can be obtained using the same formulas previously described. Further, the curve-fitting values used for the stability portion of the equation can be obtained as follows:
a. G=a midpoint of stability values according to the following formula: [((no recurrence stability median)−(yes recurrence stability median))/2)+(yes recurrence stability median)]

By incorporating the stability value into the efficacy index, the efficacy index can better reflect how effective the ablative treatment is likely to be in the long term. For example, the steadier an end effector is held during ablation, the more likely it is that the lesions formed during ablation are sufficient to prevent or correct aberrant electrical signals, thereby correcting the cardiovascular abnormality and preventing recurrence of the cardiovascular abnormality. The stability value can be reflective of a distance that an electrode delivering the ablative energy to tissue is moved during ablation at a particular site. For example, the value G=2.3 can reflect that in general an electrode should be moved less than 2.3 millimeters while delivering ablative energy. As the electrode is moved a greater distance than 2.3 millimeters, the efficacy of the treatment will decrease because it becomes less likely that the lesions formed while the electrode was moving more than 2.3 millimeters was effective.

### Symptomatic and Asymptomatic Recurrence

From data obtained during treatment of patients using the above-described medical probe for ablation of cardiac tissue, it was determined that at least 85% of patients who received ablation with at least 80% of the ablations being equal to or greater than the target contact force and at least 75% of the ablations being within the target inter-lesion distance did not experience symptomatic or asymptomatic recurrence of the treated cardiovascular abnormality within twelve months. In other examples, the percentage of patients who received ablation with at least 80% of the ablations being equal to or greater than the target contact force and at least 75% of the ablations being within the target inter-lesion distance who did not experience symptomatic or asymptomatic recurrence of the treated cardiovascular abnormality within twelve months was as high as 87%. In other examples the percentage was as high as 88%. In a specific example, 88.57% of patients who received ablation with at least 80% of the ablations being equal to or greater than the target contact force and at least 75% of the ablations being within the target inter-lesion distance did not experience symptomatic or asymptomatic recurrence of the treated cardiovascular abnormality within twelve months.

Furthermore, at least 82% of patients who received ablation with at least 70% of the ablations being equal to or greater than the target ablation index and at least 75% of the ablations being within the target inter-lesion distance did not experience symptomatic or asymptomatic recurrence of the treated cardiovascular abnormality within twelve months. In other examples, the percentage of patients who received ablation with at least 70% of the ablations being equal to or greater than the target ablation index and at least 75% of the ablations being within the target inter-lesion distance that did not experience symptomatic or asymptomatic recurrence of the treated cardiovascular abnormality was as high as 84% or 85%. In a specific example, 85.71% of patients who received ablation with at least 70% of the ablations being equal to or greater than the target ablation index and at least 75% of the ablations being within the target inter-lesion distance did not experience symptomatic or asymptomatic recurrence of the treated cardiovascular abnormality within twelve months.

The study also found that if 80% of ablations are equal to or greater than the target contact force, not considering other factors, at least 85% of patients did not experience symptomatic or asymptomatic recurrence of the treated cardiovascular abnormality within twelve months. Specifically, if 80% of ablations are equal to or greater than the target contact force, not considering other factors, approximately 86.21% of patients did not experience symptomatic or asymptomatic recurrence of the treated cardiovascular abnormality within twelve months.

If 75% of ablations were equal to or less than the target-inter lesion distance, not considering other factors, at least 85% of patients did not experience symptomatic or asymptomatic recurrence of the treated cardiovascular abnormality within twelve months. Specifically, if 75% of ablations were equal to or less than the target-inter lesion distance, not considering other factors, approximately 86.84% of patients did not experience symptomatic or asymptomatic recurrence of the treated cardiovascular abnormality within twelve months.

Furthermore, if 70% of ablations were equal to or greater than the target ablation index, not considering other factors, at least 80% of patients did not experience symptomatic or asymptomatic recurrence of the treated cardiovascular abnormality within twelve months. Specifically, if 70% of ablations were equal to or greater than the target ablation index, not considering other factors, approximately 82.88% of patients did not experience symptomatic or asymptomatic recurrence of the treated cardiovascular abnormality within twelve months.

### Symptomatic Recurrence

During the same study just described, the results specifically for patients who experienced symptomatic recurrence of the cardiovascular abnormality were reviewed (as compared to the group of patients who experienced either symptomatic or asymptomatic recurrence, or both). The results showed that, if the target thresholds were followed, patients generally had a higher chance of not experiencing symptomatic recurrence of the cardiovascular abnormality as compared to the group of patients who experienced symptomatic or asymptomatic recurrence as previously discussed. In particular, from data obtained during treatment of patients using the above-described medical probe for ablation of cardiac tissue, it was determined that at least 95% of patients who received ablation with at least 80% of the ablations being equal to or greater than the target contact force and at least 75% of the ablations being within the target inter-lesion distance did not experience symptomatic recurrence of the treated cardiovascular abnormality within twelve months. In other examples, the percentage was as high as 96% or even 97%. In one example, 97.14% of patients who received ablation with at least 80% of the ablations being equal to or greater than the target contact force and at least 75% of the ablations being within the target inter-lesion distance did not experience symptomatic recurrence of the treated cardiovascular abnormality within twelve months.

Furthermore, at least 89% of patients who received ablation with at least 80% of the ablations being equal to or greater than the target ablation index and at least 75% of the ablations being within the target inter-lesion distance did not experience symptomatic recurrence of the treated cardiovascular abnormality within twelve months. In other examples, the percentage was as high as 90%. In one example, 91.23% of patients who received ablation with at least 80% of the ablations being equal to or greater than the target ablation index and at least 75% of the ablations being within the target inter-lesion distance did not experience symptomatic recurrence of the treated cardiovascular abnormality within twelve months.

The study also found that if 80% of ablations are equal to or greater than the target contact force, not considering other factors, at least 95% of patients did not experience symptomatic recurrence of the treated cardiovascular abnormality within twelve months. In other examples, the percentage was as high as 96%. In one example, if 80% of ablations are equal to or greater than the target contact force, not considering other factors, approximately 96.55% of patients did not experience symptomatic recurrence of the treated cardiovascular abnormality within twelve months.

If 75% of ablations were equal to or less than the target-inter lesion distance, not considering other factors, at least 92% of patients did not experience symptomatic recurrence of the treated cardiovascular abnormality within twelve months. In some examples, the percentage was as high as 92%. In one example, if 75% of ablations were equal to or less than the target-inter lesion distance, not considering other factors, approximately 93.42% of patients did not experience symptomatic recurrence of the treated cardiovascular abnormality within twelve months.

Furthermore, if 80% of ablations were equal to or greater than the target ablation index, not considering other factors, at least 87% of patients did not experience symptomatic recurrence of the treated cardiovascular abnormality within twelve months. In other examples, the percentage was as high as 88%. In one example, if 80% of ablations were equal to or greater than the target ablation index, not considering other factors, approximately 89.29% of patients did not experience symptomatic recurrence of the treated cardiovascular abnormality within twelve months.

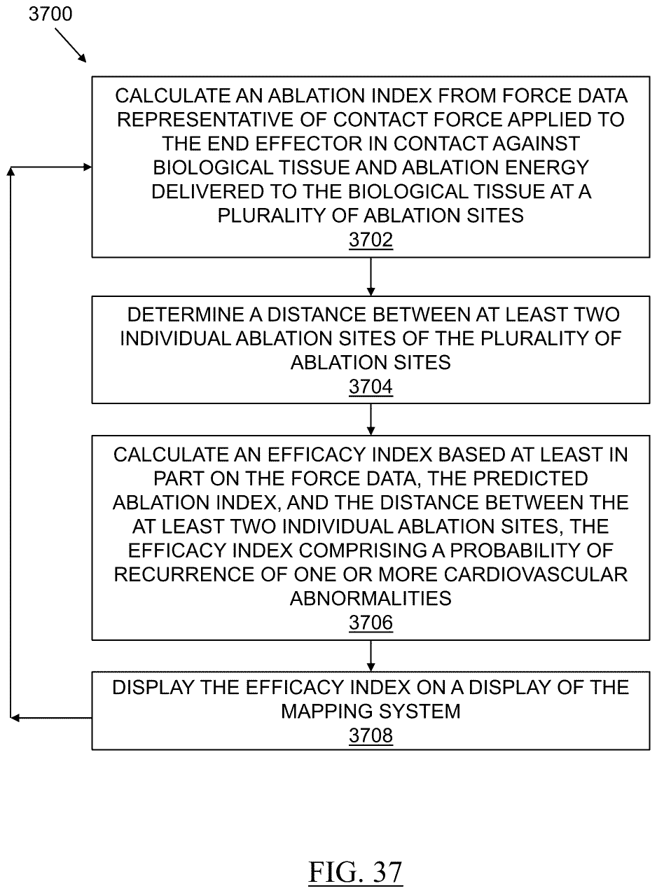

FIG. 37 illustrates a flow chart of a method 3700 of determining an efficacy index for a cardiac ablation procedure with a computerized electroanatomical mapping system that receives location and force data provided by a medical end effector disposed in a patient. The method 3700 can include calculating 3702 an ablation index from force data representative of contact force applied to the medical end effector in contact against biological tissue and ablation energy delivered to the biological tissue at a plurality of ablation sites.

The method 3700 can further include determining 3704 a distance between at least two individual ablation sites of the plurality of ablation sites and calculating 3706 an efficacy index based at least in part on the force data, the ablation index, and the distance between the at least two individual ablation sites. The efficacy index can comprise a probability of recurrence of one or more cardiovascular abnormalities. The method 3700 can also include displaying 3708 the efficacy index on a display of the mapping system.

As will be appreciated, the method 3700 can be repeated and the efficacy index updated with each new ablation site, thereby giving the physician a real time indication of the effectiveness of the ablation procedure. It will also be appreciated that the method 3700 can be modified according to the other examples described herein. Thus, although the method 3700 is shown as being completed in a particular order, the various method steps are not necessarily limited to being completed in the particular order shown and described.

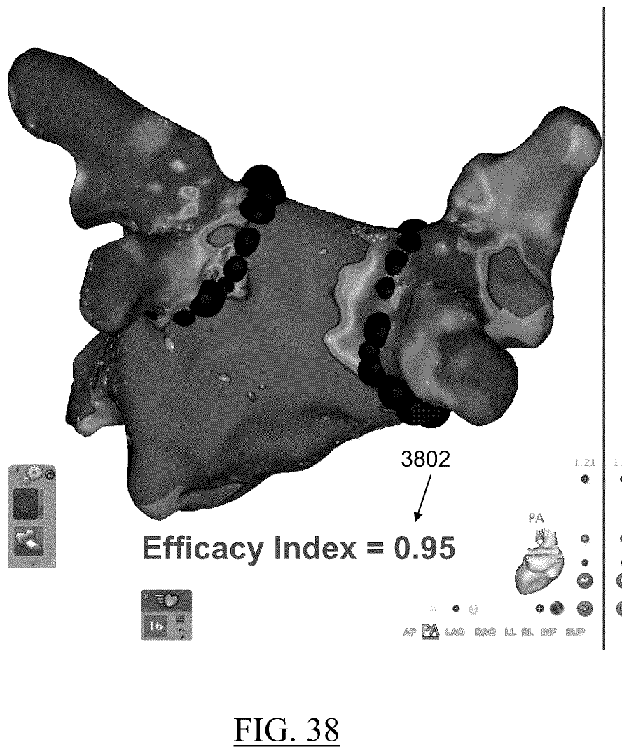

As shown in FIG. 38, the efficacy index 3802 can be displayed showing a real time calculation of the efficacy index. In the example shown in FIG. 38, the efficacy index 3802 is shown as displaying numerical value of the currently-calculate efficacy index based on the ablation sites currently formed. It will be appreciated, however, that the efficacy index can be continuously updated as more ablation sites are formed. Further, although shown as a numerical value, the efficacy index can alternatively, or in addition, be displayed as an indication of the efficacy index such as a percentage value, a symbol, a color (red, yellow, green), or other suitable indicators of the efficacy index or a combination of various indicators.

As will be appreciated, the foregoing illustrates how the efficacy index can help provide useful data to a physician in real time as they are completing an ablation procedure to help him or her determine whether the cardiac ablation is likely to effectively prevent recurrence of the particular cardiovascular abnormality.

The disclosed technology described herein are directed to the scope of the invention as described in the following clauses:

Clause 1: A method comprising: navigating a medical probe to a target location in a patient's cardiovascular system, the medical probe extending along a longitudinal axis and comprising a plurality of spines configured to bow radially outward from the longitudinal axis, the plurality of spines comprising a plurality of electrodes disposed thereon; placing at least a portion of the plurality of electrodes in contact with tissue of the patient's cardiovascular system with an indicated contact force of about 20 grams or greater; delivering, via the at least a portion of the plurality of electrodes, a plurality of electrical pulses to the tissue, the plurality of electrical pulses being configured to ablate the tissue to form a plurality of lesions having a spacing of about less than or equal to 9 millimeters between approximate centers of adjacent lesions.

Clause 2: The method of clause 1, wherein the tissue comprises at least a portion of a posterior wall of a heart and the indicated contact force comprises about 25 grams or greater.

Clause 3: The method of clause 1, wherein the tissue comprises at least a portion of a carina of a heart and the indicated contact force comprises about 20 grams or greater to about 50 grams or less.

Clause 4: The method of clause 1, wherein the tissue comprises at least a portion of an inferior wall of a heart and the indicated contact force comprises about 20 grams or greater to about 50 grams or less.

Clause 5: The method of clause 1, wherein the tissue comprises at least a portion of an anterior wall of a heart and the indicated contact force comprises about 35 grams or greater and the spacing comprises about 6 millimeters or less.

Clause 6: The method of clause 1, wherein the tissue comprises at least a portion of a ridge segment of a heart and the indicated contact force comprises about 30 grams or greater to about 50 grams or less.

Clause 7: The method of clause 1, wherein the tissue comprises at least a portion of a superior segment of a heart and the indicated contact force comprises about 30 grams or greater to about 50 grams or less.

Clause 8: The method of clause 1, wherein the tissue comprises at least a portion of a roof segment of a heart and the indicated contact force comprises about 30 grams or greater to about 50 grams or less.

Clause 9: The method of any one of clauses 5-8, wherein the plurality of lesions have a spacing of about 6 millimeters or less.

Clause 10: The method of any one of clauses 1-9, wherein the medical probe comprises a contact force sensor configured to detect the contact force and output force data.

Clause 11: The method of clause 10 further comprising: receiving or collecting (as well as storing) the force data from the contact force sensor; determining, based at least in part on the force data, a predicted ablation index of an ablation that would be caused by applying by the catheter force equal to the detected contact force for a predetermined number of electrical pulses having predetermined magnitude and predetermined duration; and outputting the predicted ablation index for display.

Clause 12: The method of clause 11, wherein, when the predicted ablation index is less than a predetermined low ablation index threshold for a given location, the method further comprises increasing the contact force and a subsequent plurality of ablation pulses.

Clause 13: The method of any one of clauses 1-11 further comprising receiving or collecting (as well as storing), via the plurality of electrodes, electrophysiological signals; and generating an electro-anatomical map using the electrophysiological signals.

Clause 14: The method of clause 13, wherein the receiving or collecting (as well as storing) electrophysiological signals and generating an electro-anatomical map are completed prior to delivering the plurality of electrical pulses to the tissue.

Clause 15: The method of clause 13, wherein the receiving or collecting (as well as storing) electrophysiological signals and generating an electro-anatomical map are completed after delivering the plurality of electrical pulses to the tissue.

Clause 16: The method of clause 15 further comprising confirming the plurality of lesions have achieved isolation of a pulmonary vein by entrance block by an adenosine challenge or an isoproterenol challenge.

Clause 17: The method of clause 15 or clause 16, further comprising: determining whether one or more acute reconnections are present; and delivering, via the at least a portion of the plurality of electrodes, a second plurality of electrical pulses to the tissue to treat the one or more acute reconnections.

Clause 18: The method of any one of clauses 1-17, wherein the method is successfully completed when pulmonary vein isolation is achieved and sustained for at least twelve months in a patient having symptomatic paroxysmal atrial fibrillation using the medical probe.

Clause 19: The method of any one of clauses 1-17, wherein the method is completed using no fluoroscopy.

Clause 20: The method of any one of clauses 1-19, wherein a primary safety endpoint of the method comprises no occurrence of one or more primary adverse events within seven days from delivering the plurality of electrical pulses to the tissue, the primary adverse events comprising: esophageal perforating complications, cardiac tamponade or perforation, death, major vascular access complication, myocardial infarction, pericarditis, heart block, phrenic near paralysis, pulmonary vein stenosis, stroke or cerebral vascular accident, thromboembolism, transient ischemic attack, pulmonary edema, or vagal nerve injury/gastroparesis.

Clause 21: The method of any one of clauses 1-20, wherein a primary effectiveness endpoint of the method comprises freedom from symptomatic or asymptomatic atrial tachyarrhythmia episodes, based on electrocardiographic data, within a time frame from 91 days to 365 days after delivering the plurality of electrical pulses to the tissue.

Clause 22: The method of clause 21, wherein the primary effectiveness endpoint further comprises freedom from one or more failure modes, the one or more failure modes comprising: failure to confirm entrance block in all pulmonary veins; repeat ablation failure; surgical treatment for atrial fibrillation, atrial tachyarrhythmia, or atrial flutter; use of a second medical probe different from the medical probe to form at least one lesion of the plurality of lesions; and the patient requiring one or more antiarrhythmic drugs after treatment.

Clause 23: The method of any one of clauses 1-22, wherein the plurality of lesions are formed without causing collateral injury to an adjacent anatomic structure.

Clause 24: The method of clause 23, wherein the adjacent anatomic structure comprises at least one of an aorta, a lung, an esophagus, bronchi, pericardium, or a phrenic nerve.

Clause 25: The method of any one of clauses 1-24, wherein the plurality of electrical pulses are configured to cause irreversible electroporation to the tissue.

Clause 26: The method of any one of clauses 1-25, wherein the plurality of electrical pulses comprise a series of short duration, high voltage, high frequency, bipolar, biphasic pulses.

Clause 27: The method of clause 26, wherein the plurality of electrical pulses comprise a first plurality of electrical pulses, the method further comprising: delivering, via the at least a portion of the plurality of electrodes, a second plurality of electrical pulses to the tissue after a predetermined period of time after delivering the first plurality of electrical pulses.

Clause 28: A medical system comprising: a medical probe extending along a longitudinal axis, the medical probe comprising: a plurality of spines configured to bow radially outward from the longitudinal axis; a plurality of electrodes disposed along the plurality of spines; and a force sensor configured to detect a contact force and output force data; a power generator; one or more processors; and a memory storing instructions that, when executed by the one or more processors, are configured to cause the medical system to: determine contact force between the medical probe and tissue of a patient's cardiovascular system; in response to determining that the contact force is indicated at about 20 grams or greater, cause the power generator to output a plurality of electrical pulses to the tissue, the plurality of electrical pulses being configured to ablate the tissue to form a plurality of lesions having a spacing of about less than or equal to 9 millimeters between approximate centers of adjacent lesions.

Clause 29: The medical system of clause 28, wherein the tissue comprises at least a portion of a posterior wall of a heart and the indicated contact force comprises about 25 grams or greater.

Clause 30: The medical system of clause 28, wherein the tissue comprises at least a portion of a carina of a heart and the indicated contact force comprises about 20 grams or greater to about 50 grams or less.

Clause 31: The medical system of clause 28, wherein the tissue comprises at least a portion of an inferior wall of a heart and the indicated contact force comprises about 20 grams or greater to about 50 grams or less.

Clause 32: The medical system of clause 28, wherein the tissue comprises at least a portion of an anterior wall of a heart and the indicated contact force comprises about 30 grams or greater.

Clause 33: The medical system of clause 28, wherein the tissue comprises at least a portion of a ridge segment of a heart and the indicated contact force comprises about 30 grams or greater.

Clause 34: The medical system of clause 28, wherein the tissue comprises at least a portion of a superior segment of a heart and the indicated contact force comprises about 30 grams or greater.

Clause 35: The medical system of clause 28, wherein the tissue comprises at least a portion of a roof segment of a heart and the indicated contact force comprises about 30 grams or greater.

Clause 36: The medical system of any one of clauses 32-35, wherein the plurality of lesions have a spacing of about 6 millimeters or less.

Clause 37: The medical system of any one of clauses 28-36 wherein the instructions, when executed by the one or more processors, are further configured to cause the system to receive, via the plurality of electrodes, electrophysiological signals; and generate an electro-anatomical map using the electrophysiological signals.

Clause 38: The medical system of any one of clauses 28-37, wherein the medical system is configured to achieve pulmonary vein isolation that is sustained for at least twelve months in a patient having symptomatic paroxysmal atrial fibrillation.

Clause 39: The medical system of any one of clauses 28-38, wherein the medical system is configured to achieve a primary safety endpoint comprising no occurrence of one or more primary adverse events, the primary adverse events comprising: esophageal perforating complications, cardiac tamponade or perforation, death, major vascular access complication, myocardial infarction, pericarditis, heart block, phrenic near paralysis, pulmonary vein stenosis, stroke or cerebral vascular accident, thromboembolism, transient ischemic attack, pulmonary edema, or vagal nerve injury/gastroparesis.

Clause 40: The medical system of any one of clauses 28-39, wherein the medical system is configured to achieve a primary effectiveness endpoint comprising freedom from symptomatic or asymptomatic atrial tachyarrhythmia episodes based on electrocardiographic data.

Clause 41: The medical system of clause 40, wherein the primary effectiveness endpoint further comprises freedom from one or more failure modes, the one or more failure modes comprising: failure to confirm entrance block in all pulmonary veins; repeat ablation failure; surgical treatment for atrial fibrillation, atrial tachyarrhythmia, or atrial flutter; use of a second medical probe different from the medical probe to form at least one lesion of the plurality of lesions; and the patient requiring one or more antiarrhythmic drugs after treatment.

Clause 42: The medical system of any one of clauses 28-41, wherein the medical system is configured to form the plurality of lesions without causing collateral injury to an adjacent anatomic structure.

Clause 43: The medical system of clause 42, wherein the adjacent anatomic structure comprises at least one of an aorta, a lung, an esophagus, bronchi, pericardium, or a phrenic nerve.

Clause 44: The medical system of any one of clauses 28-43 further comprising: determining, based at least in part on the force data, a predicted ablation index of an ablation that would be caused by applying by the catheter force equal to the detected contact force for a predetermined number of electrical pulses having predetermined magnitude and predetermined duration; and outputting the predicted ablation index for display.

Clause 45: The medical system of clause 44, wherein, when the predicted ablation index is less than a predetermined low ablation index threshold for a given location, the method further comprises increasing the contact force and a subsequent plurality of ablation pulses.

Clause 46: The medical system of any one of clauses 28-45, wherein the plurality of electrical pulses are configured to cause irreversible electroporation to the tissue.

Clause 47: The medical system of any one of clauses 28-46, wherein the plurality of electrical pulses comprises a series of short duration, high voltage, high frequency, bipolar, biphasic pulses.

Clause 48: The medical system of clause 47, wherein the plurality of electrical pulses comprises a first plurality of electrical pulses and a second plurality of electrical pulses, the power generator configured to deliver the second plurality of electrical pulses after a predetermine period of time.

Clause 49: The medical system of any one of clauses 28-48, wherein the plurality of lesions have a spacing of greater than 0 millimeters between centers of adjacent lesions

Clause 50. An electrophysiology system to provide an efficacy index of a cardiac ablation treatment, the system comprising: a processor connected to a medical probe, user interface, display, memory and storage, the processor configured to:
collect force data from the medical probe at a target location in a patient's cardiovascular system, the force data being indicative of a contact force applied to the medical probe at individual ablation sites of a plurality of ablation sites at the target location;
collect ablation data corresponding to ablative energy applied at each individual ablation site of the plurality of ablation sites;
determine a predicted ablation index;
determine a distance between at least two individual ablation sites of the plurality of ablation sites; and
output to the display an efficacy index based at least in part on the force data, the predicted ablation index, and the distance between the at least two individual ablation sites, the efficacy index comprises a probability of recurrence of one or more cardiovascular abnormalities.

Clause 51: The electrophysiology system of clause 50, wherein the medical probe extends along a longitudinal axis and comprises a plurality of spines configured to bow radially outward from the longitudinal axis, the plurality of spines comprising a plurality of electrodes disposed thereon.

Clause 52: The electrophysiology system of clause 50 or clause 51, wherein the efficacy index comprises a probability of recurrence of the one or more cardiovascular abnormalities within 6 months after treatment.

Clause 53: The electrophysiology system of clause 50 or clause 51, wherein the efficacy index comprises a probability of recurrence of the one or more cardiovascular abnormalities within 12 months after treatment.

Clause 54: The electrophysiology system of clause 50 or clause 51, wherein the efficacy index comprises a probability of recurrence of the one or more cardiovascular abnormalities within 2 years after treatment.

Clause 55: The electrophysiology system of clause 50 or clause 51, wherein the efficacy index comprises a probability of recurrence of the one or more cardiovascular abnormalities within 5 years after treatment.

Clause 56: The electrophysiology system of clause 50 or clause 51, wherein the efficacy index comprises a probability of recurrence of the one or more cardiovascular abnormalities within 10 years after treatment.

Clause 57: The electrophysiology system of any one of clauses 50-56, wherein the efficacy index (E) is determined according to the following equation: E=(1/(1+exp(−A(PF Index−B))))*(1−(1/(1+exp(−C(ILD−D)))))*(1/(1+exp(−H(CF−I)))).

Clause 58: The electrophysiology system of clause 57, wherein A comprises approximately 1.

Clause 59: The electrophysiology system of clause 57 or clause 58, wherein PF Index comprises the predicted ablation index for a given individual ablation site.

Clause 60: The electrophysiology system of any one of clauses 57-59, wherein B comprises approximately 471.5.

Clause 61: The electrophysiology system of any one of clauses 57-60, wherein C comprises approximately 1.

Clause 62: The electrophysiology system of any one of clauses 57-61, wherein ILD comprises the distance between the at least two ablation sites.

Clause 63: The electrophysiology system of any one of clauses 57-62, wherein D comprises approximately 4.7.

Clause 64: The electrophysiology system of any one of clauses 57-63, wherein H comprises approximately 1.

Clause 65: The electrophysiology system of any one of clauses 57-64, wherein CF comprises the contact force applied at corresponding individual ablation sites of the plurality of ablation sites.

Clause 66: The electrophysiology system of any one of clauses 57-65, wherein I comprises approximately 33.9.

Clause 67: The electrophysiology system of any one of clauses 50-66 further comprising collecting position data from one or more position sensors of the medical probe.

Clause 68: The electrophysiology system of clause 67 further comprising determining a stability value for each respective individual ablation site of the plurality of ablation sites.

Clause 69: The electrophysiology system of clause 68, wherein the stability value comprises a steadiness of the medical probe during application of the ablative energy applied at each individual ablation site of the plurality of ablation sites.

Clause 70: The electrophysiology system of any one of clauses 67-69, wherein the efficacy index is further determined according to the following: E=(1/(1+exp(−A(PF Index−B))))*(1−(1/(1+exp(−C(ILD−D)))))*(1−(1/(1+exp(−F(Stability−G)))))*(1/(1+exp(−H(CF−I)))).

Clause 71: The electrophysiology system of clause 70, wherein F comprises approximately 16, Stability comprises the stability value, and G comprises approximately 2.3.

Clause 72: The electrophysiology system of any one of clauses 50-71, wherein the efficacy index comprises data acquired during application of ablative energy at each individual ablation site of the plurality of ablation sites.

Clause 73: The electrophysiology system of any one of clauses 50-72, wherein the efficacy index returns a value between 0 and 1, with 1 representing a high likelihood that there will be no recurrence of the one or more cardiovascular abnormalities and 0 representing a high likelihood that there will be a recurrence of the one or more cardiovascular abnormalities.

Clause 74: The electrophysiology system of clause 73, wherein the one or more cardiovascular abnormalities comprises at least one of atrial fibrillation, atrial tachyarrhythmia, or atrial flutter.

Clause 75: The electrophysiology system of any one of clauses 50-74, wherein, when at least 80 percent of ablations during the cardiac ablation are completed with a contact force equal to or greater than a target contact force, at least 96 percent of patients experience no symptomatic recurrence of the one or more cardiovascular abnormalities.

Clause 76: The electrophysiology system of any one of clauses 50-74, wherein, when at least 80 percent of ablations during the cardiac ablation are completed with a contact force equal to or greater than a target contact force, at least 86 percent of patients experience no symptomatic or asymptomatic recurrence of the one or more cardiovascular abnormalities.

Clause 77: The electrophysiology system of any one of clauses 50-74, wherein, when at least 75 percent of ablations are completed with an inter-lesion distance equal to or less than a target distance, at least 93 percent of patients experience no symptomatic recurrence of the one or more cardiovascular abnormalities.

Clause 78: The electrophysiology system of any one of clauses 50-74, wherein, when at least 75 percent of ablations are completed with an inter-lesion distance equal to or less than a target distance, at least 86 percent of patients experience no symptomatic or asymptomatic recurrence of the one or more cardiovascular abnormalities.

Clause 79: The electrophysiology system of any one of clauses 50-74, wherein, when at least 80 percent of ablations during the cardiac ablation are completed with a predicted ablation index equal to or greater than a target predicted ablation index, at least 89 percent of patients experience no symptomatic recurrence of the one or more cardiovascular abnormalities.

Clause 80: The electrophysiology system of any one of clauses 50-74, wherein, when at least 80 percent of ablations during the cardiac ablation are completed with a predicted ablation index equal to or greater than a target predicted ablation index, at least 82 percent of patients experience no symptomatic or asymptomatic recurrence of the one or more cardiovascular abnormalities.

Clause 81: The electrophysiology system of any one of clauses 50-74, wherein, when at least 80 percent of ablations during the cardiac ablation are completed with a contact force equal to or greater than a target contact force and at least 65 percent of ablations are completed with an inter-lesion distance equal to or less than a target distance, at least 97 percent of patients experience no symptomatic recurrence of the one or more cardiovascular abnormalities.

Clause 82: The electrophysiology system of any one of clauses 50-74, wherein, when at least 80 percent of ablations during the cardiac ablation are completed with a contact force equal to or greater than a target contact force and at least 75 percent of ablations are completed with an inter-lesion distance equal to or less than a target distance, at least 88 percent of patients experience no symptomatic or asymptomatic recurrence of the one or more cardiovascular abnormalities.

Clause 83: The electrophysiology system of any one of clauses 50-74, wherein, when at least 80 percent of ablations during the cardiac ablation are completed with a predicted ablation index equal to or greater than a target predicted ablation index and at least 75 percent of ablations are completed with an inter-lesion distance equal to or less than a target distance, at least 91 percent of patients experience no symptomatic recurrence of the one or more cardiovascular abnormalities.

Clause 84: The electrophysiology system of any one of clauses 50-74, wherein, when at least 70 percent of ablations during the cardiac ablation are completed with a predicted ablation index equal to or greater than a target predicted ablation index and at least 75 percent of ablations are completed with an inter-lesion distance equal to or less than a target distance, at least 85 percent of patients experience no symptomatic or asymptomatic recurrence of the one or more cardiovascular abnormalities.

Clause 85: The electrophysiology system of any one of clauses 50-84, wherein a target distance between the at least two ablation sites of the plurality of ablation sites comprises 9 millimeters or less between approximate centers of adjacent lesions formed at the at least two ablation sites when the target location comprises a posterior portion of a patient's heart.

Clause 86: The electrophysiology system of any one of clauses 50-85, wherein a target contact force applied at the at least two ablation sites of the plurality of ablation sites comprises 20 grams or greater when the target location comprises a posterior portion of a patient's heart.

Clause 87: The electrophysiology system of any one of clauses 50-86, wherein a target predicted ablation index comprises 400 or greater when the target location comprises a posterior portion of a patient's heart.

Clause 88: The electrophysiology system of any one of clauses 50-87, wherein a target distance between the at least two ablation sites of the plurality of ablation sites comprises 6 millimeters or less between approximate centers of adjacent lesions formed at the at least two ablation sites when the target location comprises an anterior portion of a patient's heart.

Clause 89: The electrophysiology system of any one of clauses 50-88, wherein a target contact force applied at the at least two ablation sites of the plurality of ablation sites comprises 30 grams or greater when the target location comprises an anterior portion of a patient's heart.

Clause 90: The electrophysiology system of any one of clauses 50-89, wherein a target predicted ablation index comprises 500 or greater when the target location comprises an anterior portion of a patient's heart.

Clause 91: The electrophysiology system of any one of clauses 50-90, wherein the efficacy index is updated each time an ablation site is added.

Clause 92: The electrophysiology system of any one of clauses 50-91, wherein the efficacy index is automatically determined during an ablation procedure and output to a display for a physician to view.

Clause 93: A method of determining an efficacy index for a cardiac ablation procedure with a computerized electroanatomical mapping system that receives location and force data provided by a medical end effector disposed in a patient, the method comprising: calculating an ablation index from force data representative of contact force applied to the medical end effector in contact against biological tissue and the ablation energy delivered to the biological tissue at a plurality of ablation sites; determining a distance between at least two individual ablation sites of the plurality of ablation sites; calculating an efficacy index based at least in part on the force data, the ablation index, and the distance between the at least two individual ablation sites, the efficacy index comprising a probability of recurrence of one or more cardiovascular abnormalities; and displaying the efficacy index on a display of the mapping system.

Clause 94: The method of clause 93, wherein the efficacy index comprises a probability of recurrence of the one or more cardiovascular abnormalities within 6 months after treatment.

Clause 95: The method of clause 93, wherein the efficacy index comprises a probability of recurrence of the one or more cardiovascular abnormalities within 12 months after treatment.

Clause 96: The method of clause 93, wherein the efficacy index comprises a probability of recurrence of the one or more cardiovascular abnormalities within 2 years after treatment.

Clause 97: The method of clause 93, wherein the efficacy index comprises a probability of recurrence of the one or more cardiovascular abnormalities within 5 years after treatment.

Clause 98: The method of clause 93, wherein the efficacy index comprises a probability of recurrence of the one or more cardiovascular abnormalities within 10 years after treatment.

Clause 99: The method of any one of clauses 93-98, wherein the efficacy index (E) is determined according to the following equation: E=(1/(1+exp(−A(PF Index−B))))*(1−(1/(1+exp(−C(ILD−D)))))*(1/(1+exp(−H(CF−I)))).

Clause 100: The method of clause 99, wherein A comprises approximately 1.

Clause 101: The method of clause 99 or clause 100, wherein PF Index comprises the predicted ablation index for a given individual ablation site.

Clause 102: The method of any one of clauses 99-101, wherein B comprises approximately 471.5.

Clause 103: The method of any one of clauses 99-102, wherein C comprises approximately 1.

Clause 104: The method of any one of clauses 99-103, wherein ILD comprises the distance between the at least two ablation sites.

Clause 105: The method of any one of clauses 99-104, wherein D comprises approximately 4.7.

Clause 106: The method of any one of clauses 99-105, wherein H comprises approximately 1.

Clause 107: The method of any one of clauses 99-106, wherein CF comprises the contact force applied at corresponding individual ablation sites of the plurality of ablation sites.

Clause 108: The method of any one of clauses 99-107, wherein I comprises approximately 33.9.

Clause 109: The method of any one of clauses 93-108, further comprising collecting position data from one or more position sensors of the medical probe.

Clause 110: The method of clause 109, further comprising determining a stability value for each respective individual ablation site of the plurality of ablation sites.

Clause 111: The method of clause 109 or clause 110, wherein the stability value comprises a steadiness of the medical probe during application of the ablative energy applied at each individual ablation site of the plurality of ablation sites.

Clause 112: The method of any one of clauses 109-111, wherein the efficacy index is further determined according to the following: E=(1/(1+exp(−A(PF Index−B))))*(1−(1/(1+exp(−C(ILD−D)))))*(1−(1/(1+exp(−F(Stability−G)))))*(1/(1+exp(−H(CF−I)))).

Clause 113: The method of clause 112, wherein F comprises approximately 16, Stability comprises the stability value, and G comprises approximately 2.3.

Clause 114: The method of any one of clauses 93-113, wherein the efficacy index comprises data acquired during application of ablative energy at each individual ablation site of the plurality of ablation sites.

Clause 115: The method of any one of clauses 93-114, wherein the efficacy index returns a value between 0 and 1, with 1 representing a high likelihood that there will be no recurrence of the one or more cardiovascular abnormalities and 0 representing a high likelihood that there will be a recurrence of the one or more cardiovascular abnormalities.

Clause 116: The method of clause 115, wherein the one or more cardiovascular abnormalities comprises at least one of atrial fibrillation, atrial tachyarrhythmia, or atrial flutter.

Clause 117: The method of any one of clauses 93-116, wherein, when at least 80 percent of ablations during the cardiac ablation are completed with a contact force equal to or greater than a target contact force, at least 96 percent of patients experience no symptomatic recurrence of the one or more cardiovascular abnormalities.

Clause 118: The method of any one of clauses 93-116, wherein, when at least 80 percent of ablations during the cardiac ablation are completed with a contact force equal to or greater than a target contact force, at least 86 percent of patients experience no symptomatic or asymptomatic recurrence of the one or more cardiovascular abnormalities.

Clause 119: The method of any one of clauses 93-116, wherein, when at least 75 percent of ablations are completed with an inter-lesion distance equal to or less than a target distance, at least 93 percent of patients experience no symptomatic recurrence of the one or more cardiovascular abnormalities.

Clause 120: The method of any one of clauses 93-116, wherein, when at least 75 percent of ablations are completed with an inter-lesion distance equal to or less than a target distance, at least 86 percent of patients experience no symptomatic or asymptomatic recurrence of the one or more cardiovascular abnormalities.

Clause 121: The method of any one of clauses 93-116, wherein, when at least 80 percent of ablations during the cardiac ablation are completed with a predicted ablation index equal to or greater than a target predicted ablation index, at least 89 percent of patients experience no symptomatic recurrence of the one or more cardiovascular abnormalities.

Clause 122: The method of any one of clauses 93-116, wherein, when at least 80 percent of ablations during the cardiac ablation are completed with a predicted ablation index equal to or greater than a target predicted ablation index, at least 82 percent of patients experience no symptomatic or asymptomatic recurrence of the one or more cardiovascular abnormalities.

Clause 123: The method of any one of clauses 93-116, wherein, when at least 80 percent of ablations during the cardiac ablation are completed with a contact force equal to or greater than a target contact force and at least 65 percent of ablations are completed with an inter-lesion distance equal to or less than a target distance, at least 97 percent of patients experience no symptomatic recurrence of the one or more cardiovascular abnormalities.

Clause 124: The method of any one of clauses 93-116, wherein, when at least 80 percent of ablations during the cardiac ablation are completed with a contact force equal to or greater than a target contact force and at least 75 percent of ablations are completed with an inter-lesion distance equal to or less than a target distance, at least 88 percent of patients experience no symptomatic or asymptomatic recurrence of the one or more cardiovascular abnormalities.

Clause 125: The method of any one of clauses 93-116, wherein, when at least 80 percent of ablations during the cardiac ablation are completed with a predicted ablation index equal to or greater than a target predicted ablation index and at least 75 percent of ablations are completed with an inter-lesion distance equal to or less than a target distance, at least 91 percent of patients experience no symptomatic recurrence of the one or more cardiovascular abnormalities.

Clause 126: The method of any one of clauses 93-116, wherein, when at least 70 percent of ablations during the cardiac ablation are completed with a predicted ablation index equal to or greater than a target predicted ablation index and at least 75 percent of ablations are completed with an inter-lesion distance equal to or less than a target distance, at least 85 percent of patients experience no symptomatic or asymptomatic recurrence of the one or more cardiovascular abnormalities.

Clause 127: The method of any one of clauses 93-126, wherein a target distance between the at least two ablation sites of the plurality of ablation sites comprises 9 millimeters or less between approximate centers of adjacent lesions formed at the at least two ablation sites when the target location comprises a posterior portion of a patient's heart.

Clause 128: The method of any one of clauses 93-127, wherein a target contact force applied at the at least two ablation sites of the plurality of ablation sites comprises 20 grams or greater when the target location comprises a posterior portion of a patient's heart.

Clause 129: The method of any one of clauses 93-128, wherein a target predicted ablation index comprises 400 or greater when the target location comprises a posterior portion of a patient's heart.

Clause 130: The method of any one of clauses 93-129, wherein a target distance between the at least two ablation sites of the plurality of ablation sites comprises 6 millimeters or less between approximate centers of adjacent lesions formed at the at least two ablation sites when the target location comprises an anterior portion of a patient's heart.

Clause 131: The method of any one of clauses 93-130, wherein a target contact force applied at the at least two ablation sites of the plurality of ablation sites comprises 30 grams or greater when the target location comprises an anterior portion of a patient's heart.

Clause 132: The method of any one of clauses 93-131, wherein a target predicted ablation index comprises 500 or greater when the target location comprises an anterior portion of a patient's heart.

Clause 133: The method of any one of clauses 93-132, wherein the efficacy index is updated each time an ablation site is added.

Clause 134: A medical system for performing cardiac ablation, the medical system comprising: a medical probe extending along a longitudinal axis, the medical probe comprising: a plurality of spines configured to bow radially outward from the longitudinal axis; a plurality of electrodes disposed along the plurality of spines; and a force sensor configured to detect a contact force and output force data; a power generator; one or more processors; and a memory storing instructions that, when executed by the one or more processors, are configured to cause the medical system to: determine, based at least in part on the force data, the contact force between the medical probe and tissue of a patient's cardiovascular system; and in response to determining that the contact force is indicated at about 20 grams or greater, cause the power generator to output a plurality of electrical pulses to the tissue, the plurality of electrical pulses being configured to ablate the tissue to form a plurality of lesions having a spacing of about less than or equal to 9 millimeters between approximate centers of adjacent lesions.

Clause 135: The medical system of clause 134, the tissue comprises at least a portion of a posterior wall of a heart of the patient and the contact force comprises about 25 grams or greater.

Clause 136: The medical system of clause 134, the tissue comprises at least a portion of a carina of a heart of the patient and the contact force comprises about 20 grams or greater to about 50 grams or less.

Clause 137: The medical system of clause 134, wherein the tissue comprises at least a portion of an inferior wall of a heart of the patient and the contact force comprises about 20 grams or greater to about 50 grams or less.

Clause 138: The medical system of clause 134, wherein the tissue comprises at least a portion of an anterior wall of a heart of the patient and the contact force comprises about 35 grams or greater and the spacing comprises about 6 millimeters or less.

Clause 139: The medical system of clause 134, wherein the tissue comprises at least a portion of a ridge segment of a heart of the patient and the contact force comprises about 30 grams or greater to about 50 grams or less.

Clause 140: The medical system of clause 134, wherein the tissue comprises at least a portion of a superior segment of a heart of the patient and the contact force comprises about 30 grams or greater to about 50 grams or less.

Clause 141: The medical system of clause 134, wherein the tissue comprises at least a portion of a roof segment of a heart of the patient and the contact force comprises about 30 grams or greater to about 50 grams or less.

Clause 142: The medical system of clause 134, wherein the plurality of lesions comprises a spacing of about 6 millimeters or less.

Clause 143: The medical system of clause 134, the plurality of lesions comprises a spacing of about 6 millimeters or less.

Clause 144: The medical system of clause 134, the medical probe comprises a contact force sensor configured to detect the contact force and output force data.

Clause 145: The medical system of clause 144 further comprising: receiving the force data from the contact force sensor; determining, by the one or more processors, based at least in part on the force data, a predicted ablation index of an ablation that would be caused by applying, by the medical probe, a force equal to the detected contact force for a predetermined number of electrical pulses having predetermined magnitude and predetermined duration; and outputting the predicted ablation index for display.

Clause 146: The medical system of clause 145, when the predicted ablation index is less than a predetermined low ablation index threshold for a given location, increase the contact force and a subsequent plurality of ablation pulses.

Clause 147: The medical system of clause 134 further comprising receiving, via the plurality of electrodes, electrophysiological signals; and generating an electro-anatomical map using the electrophysiological signals.

Clause 148: The medical system of clause 147, the receiving electrophysiological signals and generating an electro-anatomical map are completed after outputting the plurality of electrical pulses to the tissue.

Clause 149: The medical system of clause 148, further comprising confirming the plurality of lesions have achieved isolation of a pulmonary vein by entrance block by an adenosine challenge or an isoproterenol challenge.

Clause 150: An electrophysiology system configured to provide an efficacy index of a cardiac ablation treatment, the system comprising: a processor in communication with a medical probe, an user interface, a display, a memory and a storage, the processor configured to: collect force data from the medical probe at a target location in a patient's cardiovascular system, the force data being indicative of a contact force applied to the medical probe at individual ablation sites of a plurality of ablation sites at the target location; collect ablation data corresponding to ablative energy applied at each individual ablation site of the plurality of ablation sites; determine a predicted ablation index; determine a distance between at least two individual ablation sites of the plurality of ablation sites; and output to the display an efficacy index based at least in part on the force data, the predicted ablation index, and the distance between the at least two individual ablation sites, the efficacy index comprising a probability of recurrence of one or more cardiovascular abnormalities.

Clause 151: The electrophysiology system of clause 150, wherein the efficacy index comprises a probability of recurrence of the one or more cardiovascular abnormalities within 12 months after treatment.

Clause 152: The electrophysiology system of clause 150, wherein the efficacy index (E) is determined according to the following equation: E=(1/(1+exp(−A(PF Index−B))))*(1−(1/(1+exp(−C(ILD−D)))))*(1/(1+exp(−H(CF−I)))).

Clause 153: The electrophysiology system of clause 152, wherein: A comprises approximately 1; PF Index comprises the predicted ablation index for a given individual ablation site; B comprises approximately 471.5; C comprises approximately 1; ILD comprises the distance between the at least two individual ablation sites; D comprises approximately 4.7; H comprises approximately 1; CF comprises the contact force applied at corresponding individual ablation sites of the plurality of ablation sites; and I comprises approximately 33.9.

The embodiments described above are cited by way of example, and the present invention is not limited by what has been particularly shown and described hereinabove. Rather, the scope of the invention includes both combinations and sub combinations of the various features described and illustrated hereinabove, as well as variations and modifications thereof which would occur to persons skilled in the art upon reading the foregoing description and which are not disclosed in the prior art.

What is claimed is:

1. A method of cardiac ablation comprising:
navigating a medical probe to a target location in a patient's cardiovascular system, the medical probe extending along a longitudinal axis and comprising a plurality of spines configured to bow radially outward from the longitudinal axis and converging towards the longitudinal axis, the plurality of spines comprising a plurality of electrodes disposed thereon;
placing at least a portion of the plurality of electrodes in contact with tissue of the patient's cardiovascular system with an indicated contact force of about 20 grams or greater; and
delivering, via the at least a portion of the plurality of electrodes, a plurality of electrical pulses to the tissue, the plurality of electrical pulses being configured to ablate the tissue to form a plurality of lesions having a spacing of about less than or equal to 9 millimeters between approximate centers of adjacent lesions.

2. The method of claim 1, wherein the tissue comprises at least a portion of a posterior wall of a heart and the indicated contact force comprises about 25 grams or greater.

3. The method of claim 1, wherein the tissue comprises at least a portion of a carina of a heart and the indicated contact force comprises about 20 grams or greater to about 50 grams or less.

4. The method of claim 1, wherein the tissue comprises at least a portion of an inferior wall of a heart and the indicated contact force comprises about 20 grams or greater to about 50 grams or less.

5. The method of claim 1, wherein the tissue comprises at least a portion of an anterior wall of a heart and the indicated contact force comprises about 35 grams or greater and the spacing comprises about 6 millimeters or less.

6. The method of claim 1, wherein the tissue comprises at least a portion of a ridge segment of a heart and the indicated contact force comprises about 30 grams or greater to about 50 grams or less.

7. The method of claim 1, wherein the tissue comprises at least a portion of a superior segment of a heart and the indicated contact force comprises about 30 grams or greater to about 50 grams or less.

8. The method of claim 1, wherein the tissue comprises at least a portion of a roof segment of a heart and the indicated contact force comprises about 30 grams or greater to about 50 grams or less.

9. The method of claim 1, wherein the plurality of lesions comprises a spacing of about 6 millimeters or less.

10. The method of claim 1, wherein the medical probe comprises a contact force sensor configured to detect the contact force and output force data.

11. The method of claim 10 further comprising: receiving the force data from the contact force sensor; determining, based at least in part on the force data, a predicted ablation index of an ablation that would be caused by applying, by the medical probe, a force equal to the detected contact force for a predetermined number of electrical pulses having predetermined magnitude and predetermined duration; and outputting the predicted ablation index for display.

12. The method of claim 11, wherein, when the predicted ablation index is less than a predetermined low ablation index threshold for a given location, the method further comprises increasing the contact force and a subsequent plurality of ablation pulses.

13. The method of claim 1 further comprising receiving, via the plurality of electrodes, electrophysiological signals; and generating an electro-anatomical map using the electrophysiological signals.

14. The method of claim 13, wherein the collecting electrophysiological signals and generating an electro-anatomical map are completed after delivering the plurality of electrical pulses to the tissue.

15. The method of claim 14 further comprising confirming the plurality of lesions have achieved isolation of a pulmonary vein by entrance block by an adenosine challenge or an isoproterenol challenge.

16. A medical system comprising:
a medical probe extending along a longitudinal axis, the medical probe comprising: a plurality of spines configured to bow radially outward from the longitudinal axis; a plurality of electrodes disposed along the plurality of spines; and a force sensor configured to detect a contact force and output force data;
a power generator;
one or more processors; and
a memory storing instructions that, when executed by the one or more processors, are configured to cause the medical system to:
determine, based at least in part on the force data, a contact force between the medical probe and tissue of a patient's cardiovascular system; and
in response to determining that the contact force is indicated at about 20 grams or greater, cause the power generator to output a plurality of electrical pulses to the tissue, the plurality of electrical pulses being configured to ablate the tissue to form a plurality of lesions having a spacing of about less than or equal to 9 millimeters between approximate centers of adjacent lesions.

17. An electrophysiology system configured to provide an efficacy index of a cardiac ablation treatment, the system comprising:
a processor in communication with a medical probe, user interface, display, memory and storage, the processor configured to:
collect force data from the medical probe at a target location in a patient's cardiovascular system, the force data being indicative of a contact force applied to the medical probe at individual ablation sites of a plurality of ablation sites at the target location;
collect ablation data corresponding to ablative energy applied at each individual ablation site of the plurality of ablation sites;
determine a predicted ablation index;
determine a distance between at least two individual ablation sites of the plurality of ablation sites; and
output to the display an efficacy index based at least in part on the force data, the predicted ablation index, and the distance between the at least two individual ablation sites, the efficacy index comprising a probability of recurrence of one or more cardiovascular abnormalities.

18. The electrophysiology system of claim 17, wherein the efficacy index comprises a probability of recurrence of the one or more cardiovascular abnormalities within 12 months after treatment.

19. The electrophysiology system of claim 17, wherein the efficacy index (E) is determined according to the following equation:
E=(1/(1+exp(−A(PF Index−B))))*(1−(1/(1+exp(−C(ILD−D)))))*(1/(1+exp(−H(CF−I)))).

20. The electrophysiology system of claim 19, wherein:
A comprises approximately 1;
PF Index comprises the predicted ablation index for a given individual ablation site;
B comprises approximately 471.5;
C comprises approximately 1;
ILD comprises the distance between the at least two individual ablation sites;
D comprises approximately 4.7;
H comprises approximately 1;
CF comprises the contact force applied at corresponding individual ablation sites of the plurality of ablation sites; and
I comprises approximately 33.9.
